SEQUENCE LISTING (55 sequences)

SEQ ID NO: 1 - protein, 22 aa, Severe acute respiratory syndrome coronavirus 2
SGSGPATVCGPKKSTNLVKNKC

SEQ ID NO: 2 - protein, 22 aa, Severe acute respiratory syndrome coronavirus 2
SGSGKSTNLVKNKCVNFNFNGL

SEQ ID NO: 3 - protein, 22 aa, Severe acute respiratory syndrome coronavirus 2
SGSGKCVNFNFNGLTGTGVLTE

SEQ ID NO: 4 - protein, 22 aa, Severe acute respiratory syndrome coronavirus 2
SGSGGLTGTGVLTESNKKFLPF

SEQ ID NO: 5 - protein, 22 aa, Severe acute respiratory syndrome coronavirus 2
SGSGTESNKKFLPFQQFGRDIA

SEQ ID NO: 6 - protein, 22 aa, Severe acute respiratory syndrome coronavirus 2
SGSGNFSQILPDPSKPSKRSFI

SEQ ID NO: 7 - protein, 22 aa, Severe acute respiratory syndrome coronavirus 2
SGSGPSKPSKRSFIEDLLFNKV

SEQ ID NO: 8 - protein, 22 aa, Severe acute respiratory syndrome coronavirus 2
SGSGFIEDLLFNKVTLADAGFI

SEQ ID NO: 9 - protein, 22 aa, Severe acute respiratory syndrome coronavirus 2
SGSGKVTLADAGFIKQYGDCLG

SEQ ID NO: 10 - protein, 22 aa, Severe acute respiratory syndrome coronavirus 2
SGSGFIKQYGDCLGDIAARDLI

SEQ ID NO: 11 - protein, 22 aa, Severe acute respiratory syndrome coronavirus 2
SGSGVLTPSSKRFQPFQQFGRD

SEQ ID NO: 12 - protein, 22 aa, Severe acute respiratory syndrome coronavirus 2
SGSGREVFAQVKQMYKTPTLKY

SEQ ID NO: 13 - protein, 22 aa, Severe acute respiratory syndrome coronavirus 2
SGSGQMYKTPTLKYFGGFNFSQ

SEQ ID NO: 14 - protein, 22 aa, Severe acute respiratory syndrome coronavirus 2
SGSGKYFGGFNFSQILPDPLKP

SEQ ID NO: 15 - protein, 22 aa, Severe acute respiratory syndrome coronavirus 2
SGSGSQILPDPLKPTKRSFIED

SEQ ID NO: 16 - protein, 22 aa, Severe acute respiratory syndrome coronavirus 2
SGSGKPTKRSFIEDLLFNKVTL

SEQ ID NO: 17 - protein, 9 aa, Severe acute respiratory syndrome coronavirus 2
YLQPRTFLL

SEQ ID NO: 18 - protein, 9 aa, Severe acute respiratory syndrome coronavirus 2
GVYFASTEK

SEQ ID NO: 19 - protein, 9 aa, Severe acute respiratory syndrome coronavirus 2
EPVLKGVKL

SEQ ID NO: 20 - protein, 9 aa, Severe acute respiratory syndrome coronavirus 2
VVNQNAQAL

SEQ ID NO: 21 - protein, 9 aa, Severe acute respiratory syndrome coronavirus 2
WTAGAAAYY

SEQ ID NO: 22 - protein, 16 aa, Severe acute respiratory syndrome coronavirus 2
CVNLTTRTQLPPAYTN

SEQ ID NO: 23 - protein, 16 aa, Severe acute respiratory syndrome coronavirus 2
NVTWFHAIHVSGTNGT

SEQ ID NO: 24 - protein, 18 aa, Severe acute respiratory syndrome coronavirus 2
SFSTFKCYGVSPTKLNDL

SEQ ID NO: 25 - protein, 8 aa, Severe acute respiratory syndrome coronavirus 2
ILLNKHID

SEQ ID NO: 26 - protein, 18 aa, Severe acute respiratory syndrome coronavirus 2
AFFGMSRIGMEVTPSGTW

SEQ ID NO: 27 - protein, 10 aa, Severe acute respiratory syndrome coronavirus 2
MEVTPSGTWL

SEQ ID NO: 28 - protein, 9 aa, Severe acute respiratory syndrome coronavirus 2
GMSRIGMEV

SEQ ID NO: 29 - protein, 9 aa, Severe acute respiratory syndrome coronavirus 2
ILLNKHIDA

SEQ ID NO: 30 - protein, 9 aa, Severe acute respiratory syndrome coronavirus 2
ALNTPKDHI

SEQ ID NO: 31 - protein, 17 aa, Severe acute respiratory syndrome coronavirus 2
IRQGTDYKHWPQIAQFA

SEQ ID NO: 32 - protein, 17 aa, Severe acute respiratory syndrome coronavirus 2
KHWPQIAQFAPSASAFF

SEQ ID NO: 33 - protein, 9 aa, Severe acute respiratory syndrome coronavirus 2
LALLLLDRL

SEQ ID NO: 34 - protein, 9 aa, Severe acute respiratory syndrome coronavirus 2
LLLDRLNQL

SEQ ID NO: 35 - protein, 18 aa, Severe acute respiratory syndrome coronavirus 2
LLNKHIDAYKTFPPTEPK

SEQ ID NO: 36 - protein, 9 aa, Severe acute respiratory syndrome coronavirus 2
LQLPQGTTL

SEQ ID NO: 37 - protein, 15 aa, Severe acute respiratory syndrome coronavirus 2
AQFAPSASAFFGMSR

SEQ ID NO: 38 - protein, 18 aa, Severe acute respiratory syndrome coronavirus 2
AQFAPSASAFFGMSRIGM

SEQ ID NO: 39 - protein, 15 aa, Severe acute respiratory syndrome coronavirus 2
RRPQGLPNNTASWFT

SEQ ID NO: 40 - protein, 16 aa, Severe acute respiratory syndrome coronavirus 2
YKTFPPTEPKKDKKKK

SEQ ID NO: 41 - protein, 16 aa, Severe acute respiratory syndrome coronavirus 2
GAALQIPFAMQMAYRF

SEQ ID NO: 42 - protein, 16 aa, Severe acute respiratory syndrome coronavirus 2
MAYRFNGIGVTQNVLY

SEQ ID NO: 43 - protein, 18 aa, Severe acute respiratory syndrome coronavirus 2
QLIRAAEIRASANLAATK

SEQ ID NO: 44 - protein, 9 aa, Severe acute respiratory syndrome coronavirus 2
FIAGLIAIV

SEQ ID NO: 45 - protein, 9 aa, Severe acute respiratory syndrome coronavirus 2
ALNTLVKQL

SEQ ID NO: 46 - protein, 9 aa, Severe acute respiratory syndrome coronavirus 2
LITGRLQSL

SEQ ID NO: 47 - protein, 9 aa, Severe acute respiratory syndrome coronavirus 2
NLNESLIDL

SEQ ID NO: 48 - protein, 17 aa, Severe acute respiratory syndrome coronavirus 2
QALNTLVKQLSSNFGAI

SEQ ID NO: 49 - protein, 9 aa, Severe acute respiratory syndrome coronavirus 2
ALNTTVTQL

SEQ ID NO: 50 - protein, 9 aa, Severe acute respiratory syndrome coronavirus 2
RLNEVAKNL

SEQ ID NO: 51 - protein, 9 aa, Severe acute respiratory syndrome coronavirus 2
VLNDILSRL

SEQ ID NO: 52 - protein, 9 aa, Severe acute respiratory syndrome coronavirus 2
VVFLHVTYV

SEQ ID NO: 53 - protein, 9 aa, Severe acute respiratory syndrome coronavirus 2
TIAGLIAVI

SEQ ID NO: 54 - protein, 68 aa, Unknown [sequence omitted]

SEQ ID NO: 55 - protein, 14 aa, Artificial Sequence [sequence omitted]

(19) United States
(12) Patent Application Publication
(10) Pub. No.: US 2021/0393681 A1
(43) Pub. Date: Dec. 23, 2021

(54) Treatment of SARS-CoV-2 with Dendritic Cells for Innate and/or Adaptive Immunity

(71) Applicants: Thomas E. Ichim, Oceanside, CA (US); Timothy G. Dixon, Oceanside, CA (US); James Veltmeyer, Oceanside, CA (US)

(72) Inventors: Thomas E. Ichim, Oceanside, CA (US); Timothy G. Dixon, Oceanside, CA (US); James Veltmeyer, Oceanside, CA (US)

(73) Assignee: Therapeutic Solutions International, Inc., Oceanside, CA (US)

(21) Appl. No.: 16/907,335

(22) Filed: Jun. 22, 2020

(51) Int. Cl. A61K 35/15; A61K 39/215; C12N 5/0784

(52) U.S. Cl. CPC A61K 35/15; A61K 39/215; C12N 5/0639; A61K 2039/5158; C12N 2501/22; C12N 2501/2304; C12N 2506/115

(57) ABSTRACT

Disclosed are means, methods, and compositions of matter for prophylaxis and/or treatment of SARS-CoV-2 by administration of dendritic cells in a manner and frequency sufficient to induce activation of innate and/or adaptive immune responses. In one embodiment the invention teaches administration of dendritic cells pulsed with one or more innate immune stimulants in a manner endowing said dendritic cell with ability to induce augmentation of natural killer (NK) cell number and/or activity. In another embodiment the invention teaches the use of dendritic cells stimulated with innate immune activators in a manner to allow for uptake of viral particles and presentation of viral epitopes to T cells in order to stimulate immunological activation and/or memory responses.

### SEQUENCE LISTING

The instant application contains a Sequence Listing which has been submitted electronically in ASCII format and is hereby incorporated by reference in its entirety. Said ASCII copy, created on Nov. 5, 2020, is named TSI-16907335-NP1_SL.txt and is 13,467 bytes in size.

### BACKGROUND OF THE INVENTION

At time of writing, in June 2020, there is a global pandemic occurring caused by the highly contagious coronavirus, SARS-CoV-2 (previously known as 2019-nCoV), leading to sharp rise of a pneumonia-like disease termed Coronavirus Disease 2019 (COVID-19) [1, 2]. COVID-19 presents with a high mortality rate, estimated at 3.4% by the World Health Organization [3]. The rapid spread of the virus (estimated reproductive number R₀ 2.2-3.6 [4, 5] is causing a significant surge of patients requiring intensive care. More than 1 out of 4 hospitalized COVID-19 patients have required admission to an Intensive Care Unit (ICU) for respiratory support, and a large proportion of these ICU-COVID-19 patients, between 17% and 46%, have died [6-10].

A common observation among patients with severe COVID-19 infection is an inflammatory response localized to the lower respiratory tract [11-13]. This inflammation, associated with dyspnea and hypoxemia, in some cases evolves into excessive immune response with cytokine storm, determining progression to Acute Lung Injury (ALI), Acute Respiratory Distress Syndrome (ARDS), organ failure, and death [2, 10]. Draconian measures have been put in place in an attempt to curtail the impact of the COVID-19 epidemic on population health and healthcare systems. WHO has now classified COVID-19 a pandemic [3].

At the present time, there is neither a vaccine nor specific antiviral treatments for seriously ill patients infected with COVID-19. Crucially, no options are available for those patients with rapidly progressing ARDS evolving to organ failure. Although supportive care is provided whenever possible, including mechanical ventilation and support of vital organ functions, it is insufficient in most severe cases. Therefore, there is an urgent need for novel therapies that can dampen the excessive inflammatory response in the lungs, associated with the immunopathological cytokine storm, and accelerate the regeneration of functional lung tissue in COVID-19 patients.

### SUMMARY

Preferred embodiments herein are directed to a dendritic cell capable of stimulating natural killer cell activity and/or natural killer cell number in a host, said dendritic cell generated by the steps of: a) obtaining a monocytic cell; b) treating said monocytic cell in a manner to induce differentiation along the dendritic cell lineage; and c) exposing said dendritic cell to a stimulator of innate immune functions for a sufficient time and concentration to endow said dendritic cell ability to activate NK cells.

Preferred teachings herein are directed to embodiments wherein said dendritic cell is autologous to the recipient.

Preferred teachings herein are directed to embodiments wherein said dendritic cell is allogeneic to the recipient.

Preferred teachings herein are directed to embodiments wherein said cell expresses CD40.

Preferred teachings herein are directed to embodiments wherein said cell expresses CD80.

Preferred teachings herein are directed to embodiments wherein said cell expresses CD86.

Preferred teachings herein are directed to embodiments wherein said cell expresses IL-12.

Preferred teachings herein are directed to embodiments wherein said IL-12 is produced at a concentration of at least 10 pg per one million dendritic cells.

Preferred teachings herein are directed to embodiments wherein said cell expresses IL-18.

Preferred teachings herein are directed to embodiments wherein said IL-18 is produced at a concentration of at least 10 pg per one million dendritic cells.

Preferred teachings herein are directed to embodiments wherein said dendritic cell is not adherent to plastic.

Preferred teachings herein are directed to embodiments wherein said dendritic cell is generated by culturing a monocyte in the presence of GM-CSF and interleukin-4.

Preferred teachings herein are directed to embodiments wherein said dendritic cell is generated by culturing a monocyte in the presence of GM-CSF and interleukin-13.

Preferred teachings herein are directed to embodiments wherein said monocyte is derived from peripheral blood.

Preferred teachings herein are directed to embodiments wherein said monocyte is derived from umbilical cord blood.

Preferred teachings herein are directed to embodiments wherein said monocyte is derived from mobilized peripheral blood.

Preferred teachings herein are directed to embodiments wherein said peripheral blood is mobilized by treatment of blood donor with GM-CSF.

Preferred teachings herein are directed to embodiments wherein said peripheral blood is mobilized by treatment of blood donor with G-CSF.

Preferred teachings herein are directed to embodiments wherein said peripheral blood is mobilized by treatment of blood donor with M-CSF.

Preferred teachings herein are directed to embodiments wherein said peripheral blood is mobilized by treatment of blood donor with FLT-3 ligand.

Preferred teachings herein are directed to embodiments wherein said peripheral blood is mobilized by treatment of blood donor with Mozibil.

Preferred teachings herein are directed to embodiments wherein said monocyte is derived from a pluripotent stem cell.

Preferred teachings herein are directed to embodiments wherein said monocyte is derived from a hematopoietic stem cell.

Preferred teachings herein are directed to embodiments wherein said dendritic cell is derived from a hematopoietic stem cell.

Preferred teachings herein are directed to embodiments wherein said dendritic cell is derived from a pluripotent stem cell.

Preferred teachings herein are directed to embodiments wherein said cell is cultured in a media containing one or more ingredients selected from a group comprising of: a) insulin; b) transferrin; c) linoleic acid; d) oleic acid; and e) palmitic acid.

Preferred teachings herein are directed to embodiments wherein said monocyte is first pretreated with interferon gamma in order to endow an “M1”-like phenotype.

Preferred teachings herein are directed to embodiments wherein said M1-like phenotype is said monocyte expressing higher levels of nitric oxide synthase as compared to a non-manipulated monocyte.

Preferred teachings herein are directed to embodiments wherein said M1-like phenotype is said monocyte expressing higher levels of interleukin-12 production as compared to a non-manipulated monocyte.

Preferred teachings herein are directed to embodiments wherein said M1-like phenotype is said monocyte possessing higher levels of phagocytic activity as compared to a non-manipulated monocyte.

Preferred teachings herein are directed to embodiments wherein said M1-like phenotype is said monocyte possessing lower levels of arginase activity as compared to a non-manipulated monocyte.

Preferred teachings herein are directed to embodiments wherein said monocyte is derived from bone marrow mononuclear cells.

Preferred teachings herein are directed to embodiments wherein said cells are generated from extracting monocytic cells from a tissue source.

Preferred teachings herein are directed to embodiments wherein said tissue source is adipose tissue.

Preferred teachings herein are directed to embodiments wherein said tissue source is placenta.

Preferred teachings herein are directed to embodiments wherein said tissue source is bone marrow.

Preferred teachings herein are directed to embodiments wherein said tissue source is blood or bone marrow and GM-CSF is added to said monocytic cells derived from said tissue source in the medium at a concentration of about 1-1000 U/ml.

Preferred teachings herein are directed to embodiments further comprising that when the tissue source is bone marrow and said bone marrow cells are treated with an agent capable of killing cells expressing antigens which are not expressed on dendritic precursor cells by contacting the bone marrow with antibodies specific for antigens not present on dendritic precursor cells in a medium comprising complement.

Preferred teachings herein are directed to embodiments wherein the tissue source is bone marrow and the antibodies are directed against at least one antigen selected from the group consisting of HLA antigen, antigens present on T cells, and antigens present on mature dendritic cells.

Preferred teachings herein are directed to embodiments wherein the bone marrow is cultured with GM-CSF at a concentration of about 500-1000 U/ml.

Preferred teachings herein are directed to embodiments wherein HLA-negative marrow nonlymphocytes are cultured at a concentration of about 5.times.10.sup.5 cells/cm.sup.2.

Preferred teachings herein are directed to embodiments wherein said anti-HLA antigen antibodies and anti-T cell, B cell and monocyte antibodies are selected from the group consisting of GK 1.5 anti-CD4, Ho 2, 2 anti-CD8, B21-2 anti-Ia, and RA3-3A1/6.1 anti-B220/CD45R.

Preferred teachings herein are directed to embodiments wherein culture of said mononuclear cells leads to generation of cell aggregates, and said cell aggregates are serially subcultured one to five times.

Preferred teachings herein are directed to embodiments wherein said cell aggregates are serially subcultured two to three times.

Preferred teachings herein are directed to embodiments wherein said cell aggregates are serially subcultured two times.

Preferred teachings herein are directed to embodiments wherein nonadherent cells are purified and cell clusters derived from said nonadherent cells are subcultured after from about 0.3 to 1 day and the cell aggregates are serially subcultured every 3 to 30 days.

Preferred teachings herein are directed to embodiments wherein said cell aggregates are serially subcultured every 10 to 20 days.

Preferred teachings herein are directed to embodiments wherein said cell aggregates are serially subcultured every 20 days.

Preferred teachings herein are directed to embodiments wherein said cells are cultured in a culture media comprising of RPMI-1640 media, and wherein said culture medium is supplemented with serum.

Preferred teachings herein are directed to embodiments wherein said cells are cultured in a culture media comprising of DMEM media, and wherein said culture medium is supplemented with serum.

Preferred teachings herein are directed to embodiments wherein said cells are cultured in a culture media comprising of EMEM media, and wherein said culture medium is supplemented with serum.

Preferred teachings herein are directed to embodiments wherein said cells are cultured in a culture media comprising of Iscove's media, and wherein said culture medium is supplemented with serum.

Preferred teachings herein are directed to embodiments wherein said cells are cultured in a culture media comprising of Yssel's media, and wherein said culture medium is supplemented with serum.

Preferred teachings herein are directed to embodiments wherein said cells are cultured in a culture media comprising of Alpha-MEM media, and wherein said culture medium is supplemented with serum.

Preferred teachings herein are directed to embodiments wherein said serum is fetal calf serum.

Preferred teachings herein are directed to embodiments wherein said fetal calf serum is present in the culture media at a concentration of 0.5 to 20% volume by volume.

Preferred teachings herein are directed to embodiments wherein said fetal calf serum is present in the culture media at a concentration of 1 to 17% volume by volume.

Preferred teachings herein are directed to embodiments wherein said fetal calf serum is present in the culture media at a concentration 10% volume by volume.

Preferred teachings herein are directed to embodiments wherein said concentration of GM-CSF in the medium is about 30-100 U/ml.

Preferred teachings herein are directed to embodiments wherein said concentration of GM-CSF in the medium is about 500-1000 U/ml.

Preferred teachings herein are directed to embodiments wherein said dendritic is used for providing an antigen directly or indirectly associated with SARS-CoV-2 to a host, wherein said antigen is exposed to a culture of dendritic cells, and wherein said dendritic cells exposed to said antigen transform into antigen-activated dendritic cells.

Preferred teachings herein are directed to embodiments wherein said host is human.

Preferred teachings herein are directed to embodiments wherein said SARS-CoV-2 antigen is the spike protein or epitopes derived from it.

Preferred teachings herein are directed to embodiments wherein said spike protein epitope comprises residues 274-306.

Preferred teachings herein are directed to embodiments wherein said spike protein epitope comprises residues 510-586.

Preferred teachings herein are directed to embodiments wherein said spike protein epitope comprises residues 587—teachings.

Preferred teachings herein are directed to embodiments wherein said spike protein epitope comprises residues 784-803.

Preferred teachings herein are directed to embodiments wherein said spike protein epitope comprises residues 870-893.

Preferred teachings herein are directed to embodiments wherein said spike protein epitope comprises a peptide with 75-100% homology to SGSGPATVCGPKKSTNLVKNKC (SEQ ID NO: 1)

Preferred teachings herein are directed to embodiments wherein said spike protein epitope comprises a peptide with 75-100% homology to SGSGKSTNLVKNKCVNFNFNGL (SEQ ID NO: 2)

Preferred teachings herein are directed to embodiments wherein said spike protein epitope comprises a peptide with 75-100% homology to SGSGKCVNFNFNGLTGTGVLTE (SEQ ID NO: 3)

Preferred teachings herein are directed to embodiments wherein said spike protein epitope comprises a peptide with 75-100% homology to SGSGGLTGTGVLTESNKKFLPF (SEQ ID NO: 4)

Preferred teachings herein are directed to embodiments wherein said spike protein epitope comprises a peptide with 75-100% homology to SGSGTESNKKFLPFQQFGRDIA (SEQ ID NO: 5)

Preferred teachings herein are directed to embodiments wherein said spike protein epitope comprises a peptide with 75-100% homology to SGSGNFSQILPDPSKPSKRSFI (SEQ ID NO: 6)

Preferred teachings herein are directed to embodiments wherein said spike protein epitope comprises a peptide with 75-100% homology to SGSGPSKPSKRSFIEDLLFNKV (SEQ ID NO: 7)

Preferred teachings herein are directed to embodiments wherein said spike protein epitope comprises a peptide with 75-100% homology to SGSGFIEDLLFNKVTLADAGFI (SEQ ID NO: 8)

Preferred teachings herein are directed to embodiments wherein said spike protein epitope comprises a peptide with 75-100% homology to SGSGKVTLADAGFIKQYGDCLG (SEQ ID NO: 9)

Preferred teachings herein are directed to embodiments wherein said spike protein epitope comprises a peptide with 75-100% homology to SGSGFIKQYGDCLGDIAARDLI (SEQ ID NO: 10)

Preferred teachings herein are directed to embodiments wherein said spike protein epitope comprises a peptide with 75-100% homology to SGSGVLTPSSKRFQPFQQFGRD (SEQ ID NO: 11)

Preferred teachings herein are directed to embodiments wherein said spike protein epitope comprises a peptide with 75-100% homology to SGSGREVFAQVKQMYKTPTLKY (SEQ ID NO: 12)

Preferred teachings herein are directed to embodiments wherein said spike protein epitope comprises a peptide with 75-100% homology to SGSGQMYKTPTLKYFGGFNFSQ (SEQ ID NO: 13)

Preferred teachings herein are directed to embodiments wherein said spike protein epitope comprises a peptide with 75-100% homology to SGSGKYFGGFNFSQILPDPLKP (SEQ ID NO: 14)

Preferred teachings herein are directed to embodiments wherein said spike protein epitope comprises a peptide with 75-100% homology to SGSGSQILPDPLKPTKRSFIED (SEQ ID NO: 15)

Preferred teachings herein are directed to embodiments wherein said spike protein epitope comprises a peptide with 75-100% homology to SGSGKPTKRSFIEDLLFNKVTL (SEQ ID NO: 16)

Preferred teachings herein are directed to embodiments wherein said SARS-CoV-2 antigen comprises a peptide with 75-100% homology to YLQPRTFLL (SEQ ID NO: 17).

Preferred teachings herein are directed to embodiments wherein said SARS-CoV-2 antigen comprises a peptide with 75-100% homology to GVYFASTEK (SEQ ID NO: 18).

Preferred teachings herein are directed to embodiments wherein said SARS-CoV-2 antigen comprises a peptide with 75-100% homology to EPVLKGVKL (SEQ ID NO: 19).

Preferred teachings herein are directed to embodiments wherein said SARS-CoV-2 antigen comprises a peptide with 75-100% homology to VVNQNAQAL (SEQ ID NO: 20).

Preferred teachings herein are directed to embodiments wherein said SARS-CoV-2 antigen comprises a peptide with 75-100% homology to WTAGAAAYY (SEQ ID NO: 21).

Preferred teachings herein are directed to embodiments wherein said SARS-CoV-2 antigen comprises a peptide with 75-100% homology to CVNLTTRTQLPPAYTN (SEQ ID NO: 22).

Preferred teachings herein are directed to embodiments wherein said SARS-CoV-2 antigen comprises a peptide with 75-100% homology to NVTWFHAIHVSGTNGT (SEQ ID NO: 23).

Preferred teachings herein are directed to embodiments wherein said SARS-CoV-2 antigen comprises a peptide with 75-100% homology to SFSTFKCYGVSPTKLNDL (SEQ ID NO: 24).

Preferred teachings herein are directed to embodiments wherein said SARS-CoV-2 antigen comprises a peptide with 75-100% homology to ILLNKHID (SEQ ID NO: 25).

Preferred teachings herein are directed to embodiments wherein said SARS-CoV-2 antigen comprises a peptide with 75-100% homology to AFFGMSRIGMEVTPSGTW (SEQ ID NO: 26).

Preferred teachings herein are directed to embodiments wherein said SARS-CoV-2 antigen comprises a peptide with 75-100% homology to MEVTPSGTWL (SEQ ID NO: 27).

Preferred teachings herein are directed to embodiments wherein said SARS-CoV-2 antigen comprises a peptide with 75-100% homology to GMSRIGMEV (SEQ ID NO: 28).

Preferred teachings herein are directed to embodiments wherein said SARS-CoV-2 antigen comprises a peptide with 75-100% homology to ILLNKHIDA (SEQ ID NO: 29).

Preferred teachings herein are directed to embodiments wherein said SARS-CoV-2 antigen comprises a peptide with 75-100% homology to ALNTPKDHI (SEQ ID NO: 30).

Preferred teachings herein are directed to embodiments wherein said SARS-CoV-2 antigen comprises a peptide with 75-100% homology to IRQGTDYKHWPQIAQFA (SEQ ID NO: 31).

Preferred teachings herein are directed to embodiments wherein said SARS-CoV-2 antigen comprises a peptide with 75-100% homology to KHWPQIAQFAPSASAFF (SEQ ID NO: 32).

Preferred teachings herein are directed to embodiments wherein said SARS-CoV-2 antigen comprises a peptide with 75-100% homology to LALLLLDRL (SEQ ID NO: 33).

Preferred teachings herein are directed to embodiments wherein said SARS-CoV-2 antigen comprises a peptide with 75-100% homology to LLLDRLNQL (SEQ ID NO: 34).

The dendritic cell of claim 61, wherein said SARS-CoV-2 antigen comprises a peptide with 75-100% homology to LLNKHIDAYKTFPPTEPK (SEQ ID NO: 35).

Preferred teachings herein are directed to embodiments wherein said SARS-CoV-2 antigen comprises a peptide with 75-100% homology to LQLPQGTTL (SEQ ID NO: 36).

Preferred teachings herein are directed to embodiments wherein said SARS-CoV-2 antigen comprises a peptide with 75-100% homology to AQFAPSASAFFGMSR (SEQ ID NO: 37).

Preferred teachings herein are directed to embodiments wherein said SARS-CoV-2 antigen comprises a peptide with 75-100% homology to AQFAPSASAFFGMSRIGM (SEQ ID NO: 38).

Preferred teachings herein are directed to embodiments wherein said SARS-CoV-2 antigen comprises a peptide with 75-100% homology to RRPQGLPNNTASWFT (SEQ ID NO: 39).

Preferred teachings herein are directed to embodiments wherein said SARS-CoV-2 antigen comprises a peptide with 75-100% homology to YKTFPPTEPKKDKKKK (SEQ ID NO: 40).

Preferred teachings herein are directed to embodiments wherein said SARS-CoV-2 antigen comprises a peptide with 75-100% homology to GAALQIPFAMQMAYRF (SEQ ID NO: 41).

Preferred teachings herein are directed to embodiments wherein said SARS-CoV-2 antigen comprises a peptide with 75-100% homology to MAYRFNGIGVTQNVLY (SEQ ID NO: 42).

Preferred teachings herein are directed to embodiments wherein said SARS-CoV-2 antigen comprises a peptide with 75-100% homology to QLIRAAEIRASANLAATK (SEQ ID NO: 43).

Preferred teachings herein are directed to embodiments wherein said SARS-CoV-2 antigen comprises a peptide with 75-100% homology to FIAGLIAIV (SEQ ID NO: 44).

Preferred teachings herein are directed to embodiments wherein said SARS-CoV-2 antigen comprises a peptide with 75-100% homology to ALNTLVKQL (SEQ ID NO: 45).

Preferred teachings herein are directed to embodiments wherein said SARS-CoV-2 antigen comprises a peptide with 75-100% homology to LITGRLQSL (SEQ ID NO: 46).

Preferred teachings herein are directed to embodiments wherein said SARS-CoV-2 antigen comprises a peptide with 75-100% homology to NLNESLIDL (SEQ ID NO: 47).

Preferred teachings herein are directed to embodiments wherein said SARS-CoV-2 antigen comprises a peptide with 75-100% homology to QALNTLVKQLSSNFGAI (SEQ ID NO: 48).

Preferred teachings herein are directed to embodiments wherein said SARS-CoV-2 antigen comprises a peptide with 75-100% homology to ALNTTVTQL (SEQ ID NO: 49).

Preferred teachings herein are directed to embodiments wherein said SARS-CoV-2 antigen comprises a peptide with 75-100% homology to RLNEVAKNL (SEQ ID NO: 50).

Preferred teachings herein are directed to embodiments wherein said SARS-CoV-2 antigen comprises a peptide with 75-100% homology to VLNDILSRL (SEQ ID NO: 51).

Preferred teachings herein are directed to embodiments wherein said SARS-CoV-2 antigen comprises a peptide with 75-100% homology to VVFLHVTYV (SEQ ID NO: 52).

Preferred teachings herein are directed to embodiments wherein said SARS-CoV-2 antigen comprises a peptide with 75-100% homology to TIAGLIAVI (SEQ ID NO: 53).

Preferred teachings herein are directed to embodiments wherein said natural killer cell is capable of spontaneous cytotoxicity without need for priming.

Preferred teachings herein are directed to embodiments wherein said spontaneous cytotoxicity is release of GranzymeB.

Preferred teachings herein are directed to embodiments wherein said spontaneous cytotoxicity is release of perforin.

Preferred teachings herein are directed to embodiments wherein said spontaneous cytotoxicity is TRAIL mediated killing of a target cell.

Preferred teachings herein are directed to embodiments wherein said spontaneous cytotoxicity is FAS-ligand mediated killing of a target cell.

Preferred teachings herein are directed to embodiments wherein said natural killer cells are capable of killing cells lacking one or more MHC molecules.

Preferred teachings herein are directed to embodiments wherein said natural killer cells are capable of killing virally infected cells.

Preferred teachings herein are directed to embodiments wherein said natural killer cells are capable of promoting activation of T cells.

Preferred teachings herein are directed to embodiments wherein said natural killer cells are capable of promoting generation of memory T cells.

Preferred teachings herein are directed to embodiments wherein said generated natural killer cells are made in vitro.

Preferred teachings herein are directed to embodiments wherein said natural killer cells generated in vitro are made in a cell culture medium in which an effective concentration of interleukin-21 (IL-21) is added to the NK progenitor cell when cultured with said dendritic cell, and wherein an effective concentration of interleukin-2 (IL-2) and/or interleukin-15 (IL-15) is repeatedly added to the cell cultured, and wherein feeder cells or membrane particles thereof are added to said medium, and wherein said feeder cells are B cell derived which are EBV immortalized; and wherein said expansion of NK cells in said cell culture medium is maintained for at least 3 weeks.

Preferred teachings herein are directed to embodiments wherein said effective concentration of IL-21 in said cell culture medium is between 0.1 and 1000 ng/mL.

Preferred teachings herein are directed to embodiments wherein said effective concentration of IL-2 in said cell culture system is between 1 U/mL and 5000 U/mL and/or said effective concentration of IL-15 in said cell culture system is between 0.1 and 1000 ng/mL.

Preferred teachings herein are directed to embodiments wherein said adding repeatedly feeder cells to said medium is performed between day 5 and day 16 after the previous feeder cell addition.

Preferred teachings herein are directed to embodiments wherein said expansion of NK cells in said cell culture medium is maintained for at least 5 weeks.

Preferred teachings herein are directed to embodiments wherein said NK cells in a cell culture medium comprising a population of NK cells are purified NK cells.

Preferred teachings herein are directed to embodiments wherein the starting concentration of said NK cells in a cell culture medium comprising a population of NK cells is between 20 cells/mL and 2.times.10.sup.7 cells/mL.

Preferred teachings herein are directed to embodiments wherein the starting concentration of said NK cells in a cell culture medium comprising a population of NK cells is between 20 cells/mL and 2.5.times.10.sup.4 cells/mL.

Preferred teachings herein are directed to embodiments wherein said natural killer cell activated by said dendritic cell is capable of clonal expansion.

Preferred teachings herein are directed to embodiments wherein said natural killer cell activated by said dendritic cell expresses CD56.

Preferred teachings herein are directed to embodiments wherein said natural killer cell activated by said dendritic cell expresses CD57.

Preferred teachings herein are directed to embodiments wherein said natural killer cell activated by said dendritic cell expresses NKG2D.

Preferred teachings herein are directed to embodiments wherein said natural killer cell activated by said dendritic cell expresses CD57.

Preferred teachings herein are directed to embodiments wherein said natural killer cell activated by said dendritic cell expresses CD96.

Preferred teachings herein are directed to embodiments wherein said natural killer cell activated by said dendritic cell expresses CD152.

Preferred teachings herein are directed to embodiments wherein said natural killer cell activated by said dendritic cell expresses CD223.

Preferred teachings herein are directed to embodiments wherein said natural killer cell activated by said dendritic cell expresses CD279.

Preferred teachings herein are directed to embodiments wherein said natural killer cell activated by said dendritic cell expresses CD328.

Preferred teachings herein are directed to embodiments wherein said natural killer cell activated by said dendritic cell expresses SIGLEC9.

Preferred teachings herein are directed to embodiments wherein said natural killer cell activated by said dendritic cell expresses TIGIT.

Preferred teachings herein are directed to embodiments wherein said natural killer cell activated by said dendritic cell expresses TIM-3.

Preferred teachings herein are directed to embodiments wherein said natural kill cells activated by said dendritic cells are further activated by suppression of a co-inhibitory molecule found on natural killer cells.

Preferred teachings herein are directed to embodiments wherein said co-inhibitory molecule found on natural killer cells is selected from a group comprising of: a) PD-1; b) CD96; c) CTLA-4; d) CD223; e) CD279; f) CD328; g) SIGLEC9; h) TIGIT and i) TIM-3.

Preferred teachings herein are directed to embodiments wherein interleukin-2 is administered systemically in order to expand numbers of natural killer cells in vivo.

Preferred teachings herein are directed to embodiments wherein said stimulator of said innate immune function is a composition extracted from immune cells by a dialysis method.

Preferred teachings herein are directed to embodiments wherein said composition is extracted from leukocytes.

Preferred teachings herein are directed to embodiments wherein said composition is extracted by homogenizing an immune organ.

Preferred teachings herein are directed to embodiments wherein said homogenizing of an immune organ is performed by mechanical means.

Preferred teachings herein are directed to embodiments wherein said homogenization of an immune organ is performed by ultrasonication.

Preferred teachings herein are directed to embodiments wherein said immune organ is selected from a group of organs comprising of: a) spleen; b) lymph node; c) thymus; d) liver; e) peripheral blood mononuclear cells; f) Bursa of fabricius; and g) an organ containing a higher concentration of immune cells as compared to peripheral circulation.

Preferred teachings herein are directed to embodiments wherein said immune cells are obtained from a source that is autologous, allogeneic, or xenogeneic to the recipient.

Preferred teachings herein are directed to embodiments wherein said immune cells are derived from a member of the chondrichthyans family.

Preferred teachings herein are directed to embodiments wherein said immune cells are derived from a shark.

Preferred teachings herein are directed to embodiments wherein said leukocytes are broken down into proteins, lipids, and small molecules by use of a solvent.

Preferred teachings herein are directed to embodiments wherein said leukocytes are broken down into proteins, lipids, and small molecules by use of lyophilization.

Preferred teachings herein are directed to embodiments wherein said broken down leukocytes undergo an extraction process to isolate immune stimulatory fractions.

Preferred teachings herein are directed to embodiments wherein said extraction is performed by use of dialysis.

Preferred teachings herein are directed to embodiments wherein said dialysis is performed to extract molecules less than or equal to 12 kilodaltons.

Preferred teachings herein are directed to embodiments wherein non-denaturing conditions are used for extraction of a polypeptide substance capable of stimulating toll like receptor 4 (TLR4) on dendritic cells.

Preferred teachings herein are directed to embodiments wherein said stimulation of TLR4 on said dendritic cells is assessed by production of interleukin-12.

Preferred teachings herein are directed to embodiments wherein said stimulation of TLR4 on said dendritic cells is assessed by production of TNF-alpha.

Preferred teachings herein are directed to embodiments wherein said stimulation of TLR4 on said dendritic cells is assessed by production of interleukin-1.

Preferred teachings herein are directed to embodiments wherein said stimulation of TLR4 on said dendritic cells is assessed by production of interleukin-7.

Preferred teachings herein are directed to embodiments wherein said stimulation of TLR4 on said dendritic cells is assessed by production of interleukin-15.

Preferred teachings herein are directed to embodiments wherein said stimulation of TLR4 on said dendritic cells is assessed by production of interleukin-18.

Preferred teachings herein are directed to embodiments in which activation of said dendritic cell is achieved by culture with an amino acid sequence with at least 20% homology to the following peptide sequence:

(SEQ ID NO: 54)
EFDVILKAAGANKVAVIKAVRGATGLGLKEAKDLVESAPAALKEGVSKD
DAEALKKALEEAGAEVEVK

Preferred teachings herein are directed to embodiments wherein said stimulator of innate immunity is an activator of a “danger” receptor.

Preferred teachings herein are directed to embodiments wherein said “danger” receptor is a pathogen associated molecular pattern receptor.

Preferred teachings herein are directed to embodiments wherein said danger receptor is a toll like receptor.

Preferred teachings herein are directed to embodiments wherein said toll like receptor is TLR-2.

Preferred teachings herein are directed to embodiments wherein said TLR-2 is activated by compounds selected from a group comprising of: a) Pam3cys4; b) Heat Killed Listeria monocytogenes (HKLM); and c) FSL-1.

Preferred teachings herein are directed to embodiments wherein said toll like receptor is TLR-3.

Preferred teachings herein are directed to embodiments wherein said TLR-3 is activated by Poly IC.

Preferred teachings herein are directed to embodiments wherein said TLR-3 is activated by double stranded RNA.

Preferred teachings herein are directed to embodiments wherein said double stranded RNA is of mammalian origin.

Preferred teachings herein are directed to embodiments wherein said double stranded RNA is of prokaryotic origin.

Preferred teachings herein are directed to embodiments wherein said double stranded RNA is derived from leukocyte extract.

Preferred teachings herein are directed to embodiments wherein said leukocyte extract is a heterogeneous composition derived from freeze-thawing of leukocytes, followed by dialysis for compounds less than 15 kDa.

Preferred teachings herein are directed to embodiments wherein said toll like receptor is TLR-4.

Preferred teachings herein are directed to embodiments wherein said TLR-4 is activated by lipopolysaccharide.

Preferred teachings herein are directed to embodiments wherein said TLR-4 is activated by peptide possessing at least 80 percent homology to the sequence

(SEQ ID NO: 54)
EFDVILKAAGANKVAVIKAVRGATGLGLKEAKDLVESAPAALKEGVSKD
DAEALKKALEEAGAEVEVK.

Preferred teachings herein are directed to embodiments wherein said TLR-4 is activated by HMGB-1.

Preferred teachings herein are directed to embodiments wherein said TLR-4 is activated by a peptide derived from HMGB-1.

Preferred teachings herein are directed to embodiments wherein said HMGB-1 peptide is hp91.

Preferred teachings herein are directed to embodiments wherein said toll like receptor is TLR-5.

Preferred teachings herein are directed to embodiments wherein said TLR-5 is activated by flagellin.

Preferred teachings herein are directed to embodiments wherein said toll like receptor is TLR-7.

Preferred teachings herein are directed to embodiments wherein said TLR-7 is activated by imiquimod.

Preferred teachings herein are directed to embodiments wherein said toll like receptor is TLR-8.

Preferred teachings herein are directed to embodiments wherein said TLR-8 is activated by resmiqiumod.

Preferred teachings herein are directed to embodiments wherein said toll like receptor is TLR-9

Preferred teachings herein are directed to embodiments wherein said TLR-9 is activated by CpGDNA.

Preferred teachings herein are directed to embodiments wherein said stimulator of antigen presentation is an agent capable of upregulating expression of costimulatory molecules on antigen presenting cells.

Preferred teachings herein are directed to embodiments wherein said costimulatory molecules are selected from a group comprising of: a) CD40; b) CD80; and c) CD86.

Preferred teachings herein are directed to embodiments wherein said cell is treated with an agent capable of increasing expression of costimulatory molecules prior to utilization for activation of NK cells.

Preferred teachings herein are directed to embodiments wherein said costimulatory molecule is interleukin-12.

Preferred teachings herein are directed to embodiments wherein said costimulatory molecule is CD40.

Preferred teachings herein are directed to embodiments wherein said costimulatory molecule is CD80.

Preferred teachings herein are directed to embodiments wherein said costimulatory molecule is CD86.

Preferred teachings herein are directed to embodiments wherein said agent capable of upregulating expression of costimulatory molecules is an activator of NF-kappa B.

Preferred teachings herein are directed to embodiments wherein said activator of NF-kappa B is an inhibitor of i-kappa B.

Preferred teachings herein are directed to embodiments wherein said agent capable of inducing upregulation of costimulatory molecules is an activator of the JAK-STAT pathway.

Preferred teachings herein are directed to embodiments wherein said agent capable of upregulating activity of the JAK-STAT pathway is interferon gamma.

Preferred teachings herein are directed to embodiments wherein said activator of NF-kappa B is an activator of a Pathogen Associated Molecular Pattern (PAMP) receptor.

Preferred teachings herein are directed to embodiments wherein said PAMP receptor is selected from a group comprising of:

a) MDA5; b) RIG-1; and c) NOD.

Preferred teachings herein are directed to embodiments wherein said NK cell is further activated with a “danger” signal.

Preferred teachings herein are directed to embodiments wherein said NK cell is activated with a TLR agonist.

Preferred teachings herein are directed to embodiments wherein said NK cell is activated with a PAMP agonist.

Preferred teachings herein are directed to embodiments wherein said NK cell is generated from patient monocytes.

Preferred teachings herein are directed to embodiments, wherein said NK cell is autologous to the patient in need of treatment.

Preferred teachings herein are directed to embodiments wherein said NK cell is allogeneic to the patient in need of treatment.

Preferred teachings herein are directed to embodiments wherein said NK cell is activated in vivo by administration of GM-CSF.

Preferred teachings herein are directed to embodiments wherein said NK cell is activated in vivo by administration of FLT-3L.

Preferred teachings herein are directed to embodiments wherein said dendritic cell is pulsed with inactivated SARS-CoV-2 virus.

Preferred teachings herein are directed to embodiments wherein said dendritic cell is fused with a cell infected with attenuated SARS-CoV-2 virus.

Preferred teachings herein are directed to embodiments wherein said inactivation is achieved by chemical fixation.

Preferred teachings herein are directed to embodiments wherein said inactivation is achieved by ozonation.

Preferred teachings herein are directed to embodiments wherein said dendritic cell and/or dendritic cell NK combination are administered together with an immune modulator.

Preferred teachings herein are directed to embodiments wherein said immune modulator is selected from a group comprising of: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Preferred teachings herein are directed to embodiments wherein prior to intervention a state of lymphopenia is induced in said patient in need of treatment.

Preferred teachings herein are directed to embodiments wherein said lymphopenia is sufficient to induce homeostatic expansion of lymphocytes in said patient.

Preferred teachings herein are directed to embodiments wherein said lymphopenia is sufficient to induce homeostatic proliferation of lymphocytes residing in patient in need of treatment.

Preferred teachings herein are directed to embodiments wherein said homeostatic expansion allows for an over 50% reduction in need of said lymphocytes for costimulatory signals.

Preferred teachings herein are directed to embodiments wherein said lymphopenia is achieved by irradiation.

Preferred teachings herein are directed to embodiments wherein said irradiation is total lymphoid irradiation.

Preferred teachings herein are directed to embodiments wherein said lymphopenia is induced by administration of cyclophosphamide.

Preferred teachings herein are directed to embodiments wherein said patient is treated in a manner to increased propensity of lymphocytes for activation by treatment of said patient with a lymphocyte mitogen.

Preferred teachings herein are directed to embodiments wherein said lymphocyte mitogen comprises of interleukin-2 treatment.

Preferred teachings herein are directed to embodiments wherein said lymphocyte mitogen comprises of interleukin-7 treatment.

Preferred teachings herein are directed to embodiments wherein said lymphocyte mitogen comprises of interleukin-15 treatment.

Preferred teachings herein are directed to embodiments wherein said lymphocyte mitogen comprises of interleukin-18 treatment.

### BRIEF DESCRIPTION OF THE DRAWINGS

FIG. 1A is a bar graph showing ACTIVEIMMUNE™ Does Not Alter Proliferation of HeLa Cancer Cells

FIG. 1B is a bar graph showing ACTIVEIMMUNE™ Does Not Alter Proliferation of PC-3 Cancer Cells

FIG. 1C is a bar graph showing ACTIVEIMMUNE™ Does Not Alter Proliferation of DU-145 Cancer Cells

FIG. 1D is a bar graph showing ACTIVEIMMUNE™ Does Not Alter Proliferation of 3T3 Cancer Cells

FIG. 2A is a bar graph showing ACTIVEIMMUNE™ is a costimulator of IFN-Gamma with concanavalin A

FIG. 2B is a bar graph showing ACTIVEIMMUNE™ is a costimulator of IL-4 with concanavalin A

FIG. 2C is a bar graph showing ACTIVEIMMUNE™ is a costimulator of IFN-Gamma with phytohemagglutinin.

FIG. 2D is a bar graph showing ACTIVEIMMUNE™ is a costimulator of IL-4 with phytohemagglutinin.

FIG. 3A is a bar graph showing ACTIVEIMMUNE™ stimulates IL-12 production in dendritic cells via TLR4.

FIG. 3B is a bar graph showing ACTIVEIMMUNE™ stimulates IL-10 production in dendritic cells via TLR4.

FIG. 3C is a bar graph showing ACTIVEIMMUNE™ stimulates CD80 production in dendritic cells via TLR4.

FIG. 3D is a bar graph showing ACTIVEIMMUNE™ stimulates CD86 production in dendritic cells via TLR4.

FIG. 4 is a photograph of a gel run under 10% conditions.

FIG. 5 is a bar graph showing suppression of B16 tumor growth by leukocyte extract treated dendritic cells.

FIG. 6 As seen in FIG. 7, antitumor efficacy of leukocyte extract treated dendritic cells was diminished upon the loss of natural killer cells.

### DETAILED DESCRIPTION OF THE INVENTION

The invention provides means of utilizing dendritic cells as a means of activating innate and adaptive immunity in order to protect against and/or inhibit SARS-CoV-2 and associated COVID-19 disease. In one embodiment the invention teaches administration of immature dendritic cells that are pulsed with an innate immune activator, wherein administration of dendritic cells activated with said immune activator leads to upregulation of innate immunity. In one embodiment of the invention said innate immunity is NK cell activation. In another embodiment, said innate immunity is augmentation of NK cytotoxic activity.

Unless defined differently, all technical and scientific terms used herein have the same meanings as commonly understood by one of skill in the art to which the disclosed invention belongs. In particular, the following terms and phrases have the following meaning.

“Adjuvant” refers to a substance that is capable of enhancing, accelerating, or prolonging an immune response when given with a vaccine immunogen.

“Agonist” refers to is a substance which promotes (induces, causes, enhances or increases) the activity of another molecule or a receptor. The term agonist encompasses substances which bind receptor (e.g., an antibody, a homolog of a natural ligand from another species) and substances which promote receptor function without binding thereto (e.g., by activating an associated protein).

“Antagonist” or “inhibitor” refers to a substance that partially or fully blocks, inhibits, or neutralizes a biological activity of another molecule or receptor.

“Co-administration” refers to administration of two or more agents to the same subject during a treatment period. The two or more agents may be encompassed in a single formulation and thus be administered simultaneously. Alternatively, the two or more agents may be in separate physical formulations and administered separately, either sequentially or simultaneously to the subject. The term “administered simultaneously” or “simultaneous administration” means that the administration of the first agent and that of a second agent overlap in time with each other, while the term “administered sequentially” or “sequential administration” means that the administration of the first agent and that of a second agent does not overlap in time with each other.

“Immune response” refers to any detectable response to a particular substance (such as an antigen or immunogen) by the immune system of a host vertebrate animal, including, but not limited to, innate immune responses (e.g., activation of Toll receptor signaling cascade), cell-mediated immune responses (e.g., responses mediated by T cells, such as antigen-specific T cells, and non-specific cells of the immune system), and humoral immune responses (e.g., responses mediated by B cells, such as generation and secretion of antibodies into the plasma, lymph, and/or tissue fluids). Examples of immune responses include an alteration (e.g., increase) in Toll-like receptor activation, lymphokine (e.g., cytokine (e.g., Th1, Th2 or Th17 type cytokines) or chemokine) expression or secretion, macrophage activation, dendritic cell activation, T cell (e.g., CD4+ or CD8+ T cell) activation, NK cell activation, B cell activation (e.g., antibody generation and/or secretion), binding of an immunogen (e.g., antigen (e.g., immunogenic polypolypeptide)) to an MEW molecule, induction of a cytotoxic T lymphocyte (“CTL”) response, induction of a B cell response (e.g., antibody production), and, expansion (e.g., growth of a population of cells) of cells of the immune system (e.g., T cells and B cells), and increased processing and presentation of antigen by antigen presenting cells. The term “immune response” also encompasses any detectable response to a particular substance (such as an antigen or immunogen) by one or more components of the immune system of a vertebrate animal in vitro.

An “antigen presenting cell” is any of a variety of cells capable of displaying, acquiring, or presenting at least one antigen or antigenic fragment on (or at) its cell surface.

A “dendritic cell” (DC) is an antigen presenting cell existing in vivo, in vitro, ex vivo, or in a host or subject, or which can be derived from a hematopoietic stem cell or a monocyte. Dendritic cells and their precursors can be isolated from a variety of lymphoid organs, e.g., spleen, lymph nodes, as well as from bone marrow and peripheral blood. The DC has a characteristic morphology with thin sheets (lamellipodia) extending in multiple directions away from the dendritic cell body. Typically, dendritic cells express high levels of WIC and costimulatory (e.g., B7-1 and B7-2) molecules. Dendritic cells can induce antigen specific differentiation of T cells in vitro, and are able to initiate primary T cell responses in vitro and in vivo. Dendritic cells and T cells develop from hematopoietic stem cells along divergent “differentiation pathways.” A differentiation pathway describes a series of cellular transformations undergone by developing cells in a specific lineage. T cells differentiate from lymphopoietic precursors, whereas DC differentiate from precursors of the monocytemacrophage lineage.

“Cytokines” are protein or glycoprotein signaling molecules involved in the regulation of cellular proliferation and differentiation. Cytokines involved in differentiation and regulation of cells of the immune system include various structurally related or unrelated lymphokines (e.g., granulocyte-macrophage colony stimulating factor (GM-CSF), interferons (IFNs)) and interleukins (IL-1, IL-2, etc.)

A “polynucleotide sequence” is a nucleic acid (which is a polymer of nucleotides (A,C,T,U,G, etc. or naturally occurring or artificial nucleotide analogues) or a character string representing a nucleic acid, depending on context. Either the given nucleic acid or the complementary nucleic acid can be determined from any specified polynucleotide sequence.

An “amino acid sequence” is a polymer of amino acids (a protein, polypeptide, etc.) or a character string representing an amino acid polymer, depending on context. Either the given nucleic acid or the complementary nucleic acid can be determined from any specified polynucleotide sequence.

An “antigen” is a substance which can induce an immune response in a host or subject, such as a mammal. Such an antigenic substance is typically capable of eliciting the formation of antibodies in a host or subject or generating a specific population of lymphocytes reactive with that substance. Antigens are typically macromolecules (e.g., proteins, peptides, or fragments thereof; polysaccharides or fragments thereof) that are foreign to the host. A protein antigen or peptide antigen, or fragment thereof may be termed “antigenic protein” or “antigenic peptide,” respectively.” A fragment of an antigen is termed an “antigenic fragment.” An antigenic fragment has antigenic properties and can induce an immune response as described above.

An “immunogen” refers to a substance that is capable of provoking an immune response. Examples of immunogens include, e.g., antigens, autoantigens that play a role in induction of autoimmune diseases, and tumor-associated antigens expressed on cancer cells.

The term “immunoassay” includes an assay that uses an antibody or immunogen to bind or specifically bind an antigen. The immunoassay is typically characterized by the use of specific binding properties of a particular antibody to isolate, target, and/or quantify the antigen.

A vector is a composition or component for facilitating cell transduction by a selected nucleic acid, or expression of the nucleic acid in the cell. Vectors include, e.g., plasmids, cosmids, viruses, YACs, bacteria, poly-lysine, etc. An “expression vector” is a nucleic acid construct, generated recombinantly or synthetically, with a series of specific nucleic acid elements that permit transcription of a particular nucleic acid in a host cell. The expression vector can be part of a plasmid, virus, or nucleic acid fragment. The expression vector typically includes a nucleic acid to be transcribed operably linked to apromoter.

An “epitope” is that portion or fragment of an antigen, the conformation of which is recognized and bound by a T cell receptor or by an antibody.

A “target cell” is a cell which expresses an antigenic protein or peptide or fragment thereof on a MHC molecule on its surface. T cells recognize such antigenic peptides bound to MUC molecules killing the target cell, either directly by cell lysis or by releasing cytokines which recruit other immune effector cells to the site.

An “exogenous antigen” is an antigen not produced by a particular cell. For example, and exogenous antigen can be a protein or other polypeptide not produced by the cell that can be internalized and processed by antigen presenting cells for presentation on the cell surface. Alternatively, exogenous antigens (e.g., peptides) can be externally loaded onto MHC molecules for presentation to T cells.

An “exogenous” gene or “transgene” is a gene foreign (or heterologous) to the cell, or homologous to the cell, but in a position within the host cell nucleic acid in which the genetic element is not ordinarily found. Exogenous genes can be expressed to yield exogenous polypeptides. A “transgenic” organism is one which has a transgene introduced into its genome. Such an organism is either an animal or a plant.

The term “T cell” is also referred to as T lymphocyte, and means a cell derived from thymus among lymphocytes involved in an immune response. The T cell includes any of a CD8-positive T cell (cytotoxic T cell: CTL), a CD4-positive T cell (helper T cell), a suppressor T cell, a regulatory T cell such as a controlling T cell, an effector cell, a naive T cell, a memory T cell, an .alpha..beta.T cell expressing TCR.alpha. and .beta. chains, and a .gamma..delta.T cell expressing TCR.gamma. and .delta. chains. The T cell includes a precursor cell of a T cell in which differentiation into a T cell is directed. Examples of “cell populations containing T cells” include, in addition to body fluids such as blood (peripheral blood, umbilical blood etc.) and bone marrow fluids, cell populations containing peripheral blood mononuclear cells (PBMC), hematopoietic cells, hematopoietic stem cells, umbilical blood mononuclear cells etc., which have been collected, isolated, purified or induced from the body fluids. Further, a variety of cell populations containing T cells and derived from hematopoietic cells can be used in the present invention. These cells may have been activated by cytokine such as IL-2 in vivo or ex vivo. As these cells, any of cells collected from a living body, or cells obtained via ex vivo culture, for example, a T cell population obtained by the method of the present invention as it is, or obtained by freeze preservation, can be used. The term “antibody” is meant to include both intact molecules as well as fragments thereof that include the antigen-binding site. Whole antibody structure is often given as H.sub.2L.sub.2 and refers to the fact that antibodies commonly comprise 2 light (L) amino acid chains and 2 heavy (H) amino acid chains. Both chains have regions capable of interacting with a structurally complementary antigenic target. The regions interacting with the target are referred to as “variable” or “V” regions and are characterized by differences in amino acid sequence from antibodies of different antigenic specificity. The variable regions of either H or L chains contains the amino acid sequences capable of specifically binding to antigenic targets. Within these sequences are smaller sequences dubbed “hypervariable” because of their extreme variability between antibodies of differing specificity. Such hypervariable regions are also referred to as “complementarity determining regions” or “CDR” regions. These CDR regions account for the basic specificity of the antibody for a particular antigenic determinant structure. The CDRs represent non-contiguous stretches of amino acids within the variable regions but, regardless of species, the positional locations of these critical amino acid sequences within the variable heavy and light chain regions have been found to have similar locations within the amino acid sequences of the variable chains. The variable heavy and light chains of all antibodies each have 3 CDR regions, each non-contiguous with the others (termed L1, L2, L3, H1, H2, H3) for the respective light (L) and heavy (H) chains. The antibodies disclosed according to the invention may also be wholly synthetic, wherein the polypeptide chains of the antibodies are synthesized and, possibly, optimized for binding to the polypeptides disclosed herein as being receptors. Such antibodies may be chimeric or humanized antibodies and may be fully tetrameric in structure, or may be dimeric and comprise only a single heavy and a single light chain. The term “effective amount” or “therapeutically effective amount” means a dosage sufficient to treat, inhibit, or alleviate one or more symptoms of a disease state being treated or to otherwise provide a desired pharmacologic and/or physiologic effect, especially enhancing T cell response to a selected antigen. The precise dosage will vary according to a variety of factors such as subject-dependent variables (e.g., age, immune system health, etc.), the disease, and the treatment being administered. The terms “individual”, “host”, “subject”, and “patient” are used interchangeably herein, and refer to a mammal, including, but not limited to, primates, for example, human beings, as well as rodents, such as mice and rats, and other laboratory animals.

The term “treatment regimen” refers to a treatment of a disease or a method for achieving a desired physiological change, such as increased or decreased response of the immune system to an antigen or immunogen, such as an increase or decrease in the number or activity of one or more cells, or cell types, that are involved in such response, wherein said treatment or method comprises administering to an animal, such as a mammal, especially a human being, a sufficient amount of two or more chemical agents or components of said regimen to effectively treat a disease or to produce said physiological change, wherein said chemical agents or components are administered together, such as part of the same composition, or administered separately and independently at the same time or at different times (i.e., administration of each agent or component is separated by a finite period of time from one or more of the agents or components) and where administration of said one or more agents or components achieves a result greater than that of any of said agents or components when administered alone or in isolation.

The term “anergy” and “unresponsiveness” includes unresponsiveness to an immune cell to stimulation, for example, stimulation by an activation receptor or cytokine. The anergy may occur due to, for example, exposure to an immune suppressor or exposure to an antigen in a high dose. Such anergy is generally antigen-specific, and continues even after completion of exposure to a tolerized antigen. For example, the anergy in a T cell and/or NK cell is characterized by failure of production of cytokine, for example, interleukin (IL)-2. The T cell anergy and/or NK cell anergy occurs in part when a first signal (signal via TCR or CD-3) is received in the absence of a second signal (costimulatory signal) upon exposure of a T cell and/or NK cell to an antigen. The term “enhanced function of a T cell”, “enhanced cytotoxicity” and “augmented activity” means that the effector function of the T cell and/or NK cell is improved. The enhanced function of the T cell and/or NK cell, which does not limit the present invention, includes an improvement in the proliferation rate of the T cell and/or NK cell, an increase in the production amount of cytokine, or an improvement in cytotoxity. Further, the enhanced function of the T cell and/or NK cell includes cancellation and suppression of tolerance of the T cell and/or NK cell in the suppressed state such as the anergy (unresponsive) state, or the rest state, that is, transfer of the T cell and/or NK cell from the suppressed state into the state where the T cell and/or NK cell responds to stimulation from the outside.

The term “expression” means generation of mRNA by transcription from nucleic acids such as genes, polynucleotides, and oligonucleotides, or generation of a protein or a polypeptide by transcription from mRNA. Expression may be detected by means including RT-PCR, Northern Blot, or in situ hybridization, “Suppression of expression” refers to a decrease of a transcription product or a translation product in a significant amount as compared with the case of no suppression. The suppression of expression herein shows, for example, a decrease of a transcription product or a translation product in an amount of 30% or more, preferably 50% or more, more preferably 70% or more, and further preferably 90% or more.

In one embodiment of the invention, immunization to viruses of the same type the patient is suffering from is provided prior to cytotoxic, or immunogenic cell death induction of the virus. Immunization of the patient may be performed using known means in the art, using suitable adjuvants. Assessment of immunity is performed by quantifying reactivity of T cells or B cells in response to protein antigens or derivatives thereof, derivatives including peptide antigens or other antigenic epitopes. Responses may be assessed in terms of proliferative responses, cytokine release, antibody responses, or generation of cytotoxic T cells. Methods of assessing said responses are well known in the art. In a preferred embodiment, antibody responses are assessed to a panel of virus associated proteins subsequent to immunization of patient. Antibody responses are utilized to guide which peptides will be utilized for prior immunization. For example, if a patient is immunized with viral antigen on a weekly basis, the subsequent assessment of antibody responses is performed at approximately 1-3 months after initiation of immunization. Protocols for immunization include weekly, biweekly, or monthly. Assessment of antibody responses is performed utilizing standard enzyme linked immunosorbent (ELISA) assay.

“Transfection” refers to the process by which an exogenous DNA sequence is introduced into a eukaryotic host cell. Transfection (or transduction) can be achieved by any one of a number of means including electroporation, microinjection, gene gun delivery, retroviral infection, lipofection, superfection and the like. A “parental” cell, or organism, is an untransfected member of the host species giving rise to a transgenic cell, or organism.

The term “subject” or “host” as used herein includes, but is not limited to, an organism or animal; a mammal, including, e.g., a human, non-human primate (e.g., monkey), mouse, pig, cow, goat, rabbit, rat, guinea pig, hamster, horse, monkey, sheep, or other non-human mammal; a non-mammal, including, e.g., a non-mammalian vertebrate, such as a bird (e.g., a chicken or duck) or a fish, and a non-mammalian invertebrate.

The term “pharmaceutical composition” means a composition suitable for pharmaceutical use in a subject, including an animal or human. A pharmaceutical composition generally comprises an effective amount of an active agent and a pharmaceutically acceptable carrier.

The term “effective amount” means a dosage or amount sufficient to produce a desired result. The desired result may comprise an objective or subjective improvement in the recipient of the dosage or amount.

A “prophylactic treatment” is a treatment administered to a subject who does not display signs or symptoms of a disease, pathology, or medical disorder, or displays only early signs or symptoms of a disease, pathology, or disorder, such that treatment is administered for the purpose of diminishing, preventing, or decreasing the risk of developing the disease, pathology, or medical disorder. A prophylactic treatment functions as a preventative treatment against a disease or disorder. A “prophylactic activity” is an activity of an agent, such as a nucleic acid, vector, gene, polypeptide, protein, antigen or portion or fragment thereof, substance, or composition thereof that, when administered to a subject who does not display signs or symptoms of pathology, disease or disorder, or who displays only early signs or symptoms of pathology, disease, or disorder, diminishes, prevents, or decreases the risk of the subject developing a pathology, disease, or disorder. A “prophylactically useful” agent or compound (e.g., nucleic acid or polypeptide) refers to an agent or compound that is useful in diminishing, preventing, treating, or decreasing development of pathology, disease or disorder.

A “therapeutic treatment” is a treatment administered to a subject who displays symptoms or signs of pathology, disease, or disorder, in which treatment is administered to the subject for the purpose of diminishing or eliminating those signs or symptoms of pathology, disease, or disorder. A “therapeutic activity” is an activity of an agent, such as a nucleic acid, vector, gene, polypeptide, protein, antigen or portion or fragment thereof, substance, or composition thereof, that eliminates or diminishes signs or symptoms of pathology, disease or disorder, when administered to a subject suffering from such signs or symptoms. A “therapeutically useful” agent or compound (e.g., nucleic acid or polypeptide) indicates that an agent or compound is useful in diminishing, treating, or eliminating such signs or symptoms of a pathology, disease or disorder.

As used herein, an “antibody” refers to a protein comprising one or more polypeptides substantially or partially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (e.g., antibody) structural unit comprises a tetramer. Each tetramer is composed of two identical pairs of polypeptide chains, each pair having one “light” (about 25 kD) and one “heavy” chain (about 5070 kD). The N-terminus of each chain defines a variable region of about 100 to 110 or more amino acids primarily responsible for antigen recognition. The terms variable light chain (VL) and variable heavy chain (VH) refer to these light and heavy chains, respectively. Antibodies exist as intact immunoglobulins or as a number of well characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region thereby converting the (Fab′)2 dimer into an Fab′ monomer. The Fab′ monomer is essentially an Fab with part of the hinge region (see Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Antibodies include single chain antibodies, including single chain Fv (sFv) antibodies, in which a variable heavy and a variable light chain are joined together (directly or through a peptide linker) to form a continuous polypeptide.

An “antigen-binding fragment” of an antibody is a peptide or polypeptide fragment of the antibody which binds an antigen. An antigen-binding site is formed by those amino acids of the antibody which contribute to, are involved in, or affect the binding of the antigen. See Scott, T. A. and Mercer, E. I., CONCISE ENCYCLOPEDIA: BIOCHEMISTRY AND MOLECULAR BIOLOGY (de Gruyter, 3.sup.rd e. 1997)(hereinafter “Scott, CONCISE ENCYCLOPEDIA”) and Watson, J. D. et al., RECOMBINANT DNA (2.sup.nd ed. 1992) (hereinafter “Watson, RECOMBINANT DNA”), each of which is incorporated herein by reference in its entirety for all purposes.

A nucleic acid or polypeptide is “recombinant” when it is artificial or engineered, or derived from an artificial or engineered protein or nucleic acid. The term “recombinant” when used with reference e.g., to a cell, nucleotide, vector, or polypeptide typically indicates that the cell, nucleotide, or vector has been modified by the introduction of a heterologous (or foreign) nucleic acid or the alteration of a native nucleic acid, or that the polypeptide has been modified by the introduction of a heterologous amino acid, or that the cell is derived from a cell so modified. Recombinant cells express nucleic acid sequences (e.g., genes) that are not found in the native (non-recombinant) form of the cell or express native nucleic acid sequences (e.g., genes) that would be abnormally expressed under-expressed, or not expressed at all. The term “recombinant nucleic acid” (e.g., DNA or RNA) molecule means, for example, a nucleotide sequence that is not naturally occurring or is made by the combatant (for example, artificial combination) of at least two segments of sequence that are not typically included together, not typically associated with one another, or are otherwise typically separated from one another. A recombinant nucleic acid can comprise a nucleic acid molecule formed by the joining together or combination of nucleic acid segments from different sources and/or artificially synthesized. The term “recombinantly produced” refers to an artificial combination usually accomplished by either chemical synthesis means, recursive sequence recombination of nucleic acid segments or other diversity generation methods (such as, e.g., shuffling) of nucleotides, or manipulation of isolated segments of nucleic acids, e.g., by genetic engineering techniques known to those of ordinary skill in the art. “Recombinantly expressed” typically refers to techniques for the production of a recombinant nucleic acid in vitro and transfer of the recombinant nucleic acid into cells in vivo, in vitro, or ex vivo where it may be expressed or propagated. A “recombinant polypeptide” or “recombinant protein” usually refers to polypeptide or protein, respectively, that results from a cloned or recombinant gene or nucleic acid.

In one embodiment, the invention provides methods for use of newly differentiated dendritic cells as a means of immune stimulation in a patient suffering from COVID-19. The differentiation of mononuclear cells or monocytes, particularly monocytes derived from peripheral blood or bone marrow, into dendritic cells is known in the art. In one embodiment, cells are treated with/or cultured in interleukin-4 (IL-4), granulocyte macrophage colony stimulating factor (GM-CSF), and a culture medium supplemented with insulin, transferrin, and various lipids, including linoleic acid, oleic acid, and palmitic acid. In some embodiments, the dendritic cells described in the invention are pretreated with hypoxia to enhance migration activity as comparted to conventional dendritic cells which do not possess as efficient migrational activity. That said, conventional dendritic cells may also be utilized within the scope of the invention, in which said cells are administered in a manner to activate innate and/or adaptive immunity to SARS-CoV-2. In one embodiment, the monocytic cells are cultured in a culture medium containing Iscove's modified Dulbecco's medium (IMDM). In some such embodiments, the IMDM is further supplemented with insulin, human transferrin, linoleic acid, oleic acid, palmitic acid, bovine serum albumin, and 2-amino ethanol. The medium may also be supplemented with IL-4 and GM-CSF (granulocyte-macrophage colony stimulating factor). In a preferred embodiment, the culture medium is Yssel's medium. According to the skill of one specialized in the art, modifications may be made, for example, such media may also be supplemented with fetal bovine serum, glutamine, penicillin, and streptomycin.

In one embodiment of the invention, the monocytes used for the practice of the invention re derived from a human or non-human animal by using various methods, e.g., by leukopharesis or bone marrow aspiration. In some embodiments, a source of monocytes is depleted of alternative cell types by negative depletion of T, B and NK (natural killer) cells from density gradient preparations of mononuclear cells. In one embodiment, mononuclear cells are derived from buffy coat preparations of peripheral blood. In a preferred embodiment, depletion of T, B, and NK cells is performed using immunomagnetic beads. The invention further provides methods for the maturation of dendritic cells in a comprising culturing the dendritic cells in medium containing various activation signals such as toll like receptor agonists, anti-CD40 monoclonal antibody (mAb) and various inflammatory conditions. In some embodiments dendritic cells are pulsed with SARS-CoV-2 peptide and/or viral lysates to induce immunity.

In some embodiments, the DC of the invention are transfected with exogenous DNA molecules which encode one or more antigens, thereby producing DC which preferentially present one or more antigens of interest. Alternatively, at least one antigen may be externally loaded by supplying the DC cell with a source of exogenous peptide. In addition, the invention provides for methods for inducing an immune response in a subject, comprising administering an antigen presenting cell which activates innate immunity. The invention also provides for cell cultures containing monocytes, dendritic cells, and/or partially differentiated cells committed to a monocyte-dendritic cell differentiation pathway. In a preferred embodiment, any or all of these cells are present in Yssel's medium supplemented with IL-4 and GM-CSF.

In one embodiment, the invention was developed to address multiple aspects of the immune system in order to augment possibility of increasing overall survival of a patient suffering from COVID-19. Specifically, it is known from studies of immune modulators that recruitment of multiple arms of the immune system associates with increased efficacy. For example, it is known that natural killer cells play an important role in immune destruction of viruses [14-20]. A clinical trial demonstrated that patients who possess elevated levels of natural killer cell inhibitory proteins (soluble NKG2D ligands) demonstrated lower responses to checkpoint inhibitors [21]. Indeed this should not be surprising since studies show that NK cell infiltration of tumors induces upregulation of antigen presentation in an interferon gamma associated manner, which renders tumor cells sensitive to T cell killing [22]. This patent covers, in part, the application of checkpoint inhibitors together with dendritic cells and/or NK cell therapy for induction of immunological responses to SARS-CoV-2. Another example of the potency of combining immunotherapies is the example of Herceptin, in which approximately 1 out of 4 patients with the HER2neu antigen respond to treating. Interestingly it was found that lack of responsiveness correlates with inhibited NK cell activity [23-25]. Indeed, animal experiments demonstrate augmentation of Herceptin activity by stimulators of NK cells such as Poly (IC) and IL-12 [26, 27]. The current invention aims to integrate the main arms of the immune system so as to achieve a synergistic induction of anticancer immunity. Accordingly, the invention provides the utilization of dendritic cells with or without NK cells as adjuvants for various therapies clinically utilized against COVID-19 including vaccines, sera from patients who have entered remission, ivermectin, hydoxychloroquine, remdesivir, and other agents known in the art.

In one embodiment of the invention, immune modulatory agents are administered together with dendritic cells in order to enhance immune activation. In this particular embodiment, allogeneic dendritic cells are utilized. In one specific means of practicing the invention, allogeneic dendritic cells are generated from cord blood. Together with allogeneic dendritic cells, various immune modulators, listed below, may be utilized: aid agents are known in the art and include 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; b eta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levami sole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepri stone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anti-cancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

In some embodiments of the invention, natural killer cells are provided having reduced or absent checkpoint inhibitory receptor function, which have been cultured to express low levels of these molecules, or they have been manipulated through means known in the art such as induction of RNA interference, utilization of antisense oligonucleotides, administration of ribozymes, or used of gene editing. Preferably, these receptors are specific checkpoint inhibitory receptors. Preferably still, these checkpoint inhibitory receptors are one or more or all of CD96 (TACTILE), CD152 (CTLA4), CD223 (LAG-3), CD279 (PD-1), CD328(SIGLEC7), SIGLEC9, TIGIT and/or TIM-3. In other embodiments, NK cells are provided in which one or more inhibitory receptor signaling pathways are knocked out or exhibit reduced function—the result again being reduced or absent inhibitory receptor function. For example, signaling pathways mediated by SHP-1, SHP-2 and/or SHIP are knocked out by genetic modification of the cells. The resulting NK cells exhibit improved cytotoxicity and are of greater use therefore in cancer therapy, especially blood cancer therapy, in particular treatment of leukemias and multiple myeloma.

For killing of coronavirus, and/or for the stimulation of immunity against said coronavirus, NK cells are generated which possess enhanced therapeutic activity. In an embodiment, the genetic modification of the NK cells occurs before the cell has differentiated into an NK cell. For example, pluripotent stem cells (e.g. iPSCs) can be genetically modified to lose the capacity to express one or more checkpoint inhibitory receptors. The modified iPSCs are then differentiated to produce genetically modified NK cells with increased cytotoxicity. The same can be performed for hemapoietic stem cells, in which they can be gene edited to lack expression of immune inhibitory genes. It is preferred to reduce function of checkpoint inhibitory receptors over other inhibitory receptors, due to the expression of the former following NK cell activation. The normal or ‘ classical’ inhibitory receptors, such as the majority of the KIR family, NKG2A and LIR-2, bind MHC class I and are therefore primarily involved in reducing the problem of self-targeting. Preferably, therefore, checkpoint inhibitory receptors are knocked out. Reduced or absent function of these receptors according to the invention prevents cancer cells from suppressing immune effector function (which might otherwise occur if the receptors were fully functional). Thus a key advantage of these embodiments of the invention lies in NK cells that are less susceptible to suppression of their cytotoxic activities by cancer cells; as a result they are useful in cancer treatment. As used herein, references to inhibitory receptors generally refer to a receptor expressed on the plasma membrane of an immune effector cell, e.g. a NK cell, whereupon binding its complementary ligand resulting intracellular signals are responsible for reducing the cytotoxicity of said immune effector cell. These inhibitory receptors are expressed during both ‘resting’ and ‘activated’ states of the immune effector cell and are often associated with providing the immune system with a ‘self-tolerance’ mechanism that inhibits cytotoxic responses against cells and tissues of the body. An example is the inhibitory receptor family ‘KIR’ which are expressed on NK cells and recognize MHC class I expressed on healthy cells of the body. Also as used herein, checkpoint inhibitory receptors are usually regarded as a subset of the inhibitory receptors above. Unlike other inhibitory receptors, however, checkpoint inhibitory receptors are expressed at higher levels during prolonged activation and cytotoxicity of an immune effector cell, e.g. a NK cell. This phenomenon is useful for dampening chronic cytotoxicity at, for example, sites of inflammation. Examples include the checkpoint inhibitory receptors PD-1, CTLA-4 and CD96, all of which are expressed on NK cells. The invention hence also provides a virus killing NK cell lacking a gene encoding a checkpoint inhibitory receptor selected from CD96 (TACTILE), CD152 (CTLA4), CD223 (LAG-3), CD279 (PD-1), CD328 (SIGLEC7), SIGLEC9, TIGIT and TIM-3. Said virus killing NK cell lacking a gene can refer to either a full or partial deletion, mutation or otherwise that results in no functional gene product being expressed. In embodiments, the NK cell lacks genes encoding two or more of the inhibitory receptors.

Combination of polyvalent vaccines with other cellular therapies as the initial poly-immunogenic composition is envisioned within the context of the invention. In one embodiment cellular lysates of virus and/or virally infected cells are loaded into dendritic cells. In one embodiment the invention provides a means of generating a population of cells with viral killing ability that are polyvalently reactive, to which focus is added by subsequent peptide specific vaccination. The generation of cytotoxic lymphocytes may be performed, in one embodiment by extracted 50 ml of peripheral blood from a cancer patient and peripheral blood mononuclear cells (PBMC) are isolated using the Ficoll Method. PBMC are subsequently resuspended in 10 ml AIM-V media and allowed to adhere onto a plastic surface for 2-4 hours. The adherent cells are then cultured at 37° C. in AIM-V media supplemented with 1,000 U/mL granulocyte-monocyte colony-stimulating factor and 500 U/mL IL-4 after non-adherent cells are removed by gentle washing in Hanks Buffered Saline Solution (HBSS). Half of the volume of the GM-CSF and IL-4 supplemented media is changed every other day. Immature DCs are harvested on day 7. In one embodiment said generated DC are used to stimulate T cell and NK cell viral killing activity by pulsing with autologous viral lysate. Specifically, generated DC may be further purified from culture through use of flow cytometry sorting or magnetic activated cell sorting (MACS), or may be utilized as a semi-pure population. DC pulsed with viral epitopes or lysate may be added into said patient in need of therapy with the concept of stimulating NK and T cell activity in vivo, or in another embodiment may be incubated in vitro with a population of cells containing T cells and/or NK cells. In one embodiment DC are exposed to agents capable of stimulating maturation in vitro and rendering them resistant to virally-derived inhibitory compounds such as arginase byproducts. Specific means of stimulating in vitro maturation include culturing DC or DC containing populations with a toll like receptor agonist. Another means of achieving DC maturation involves exposure of DC to TNF-alpha at a concentration of approximately 20 ng/mL. In order to activate T cells and/or NK cells in vitro, cells are cultured in media containing approximately 1000 IU/ml of interferon gamma. Incubation with interferon gamma may be performed for the period of 2 hours to the period of 7 days. Preferably, incubation is performed for approximately 24 hours, after which T cells and/or NK cells are stimulated via the CD3 and CD28 receptors. One means of accomplishing this is by addition of antibodies capable of activating these receptors. In one embodiment approximately, 2 ug/ml of anti-CD3 antibody is added, together with approximately 1 ug/ml anti-CD28. In order to promote survival of T cells and NK cells, was well as to stimulate proliferation, a T cell/NK mitogen may be used. In one embodiment the cytokine IL-2 is utilized. Specific concentrations of IL-2 useful for the practice of the invention are approximately 500 u/mL IL-2. Media containing IL-2 and antibodies may be changed every 48 hours for approximately 8-14 days. In one particular embodiment DC are included to said T cells and/or NK cells in order to endow cytotoxic activity towards virally infected cells. In a particular embodiment, inhibitors of caspases are added in the culture so as to reduce rate of apoptosis of T cells and/or NK cells. Generated cells can be administered to a subject intradermally, intramuscularly, subcutaneously, intraperitoneally, intraarterially, intravenously (including a method performed by an indwelling catheter), or into an afferent lymph vessel. The immune response of the patient treated with these cytotoxic cells is assessed utilizing a variety of antigens found in virally infected cells. When cytotoxic or antibody, or antibody associated with complement fixation are recognized to be upregulated in the cancer patient, subsequent immunizations are performed utilizing peptides to induce a focusing of the immune response.

In another embodiment DC are generated from leukocytes of patients by leukopheresis. Numerous means of leukopheresis are known in the art. In one example, a Frenius Device (Fresenius Com.Tec) is utilized with the use of the MNC program, at approximately 1500 rpm, and with a P1Y kit. The plasma pump flow rates are adjusted to approximately 50 mL/min. Various anticoagulants may be used, for example ACD-A. The Inlet/ACD Ratio may be ranged from approximately 10:1 to 16:1. In one embodiment approximately 150 mL of blood is processed. The leukopheresis product is subsequently used for initiation of dendritic cell culture. In order to generates a peripheral blood mononuclear cells from leukopheresis product, mononuclear cells are isolated by the Ficoll-Hypaque density gradient centrifugation. Monocytes are then enriched by the Percoll hyperosmotic density gradient centrifugation followed by two hours of adherence to the plate culture. Cells are then centrifuged at 500 g to separate the different cell populations. Adherent monocytes are cultured for 7 days in 6-well plates at 2×106 cells/mL RMPI medium with 1% penicillin/streptomycin, 2 mM L-glutamine, 10% of autologous, 50 ng/mL GM-CSF and 30 ng/mL IL-4. On day 6 immature dendritic cells are pulsed with viral antigen. Pulsing may be performed by incubation of lysates with dendritic cells, or may be generated by fusion of immature dendritic cells with virally infected cells. Means of generating hybridomas or cellular fusion products are known in the art and include electrical pulse mediated fusion, or stimulation of cellular fusion by treatment with polyethelene glycol. On day 7, the immature DCs are then induced to differentiate into mature DCs by culturing for 48 hours with 30 ng/mL interferon gamma (IFN-γ). During the course of generating DC for clinical purposes, microbiologic monitoring tests are performed at the beginning of the culture, on the fifth day and at the time of cell freezing for further use or prior to release of the dendritic cells. Administration of viral pulsed dendritic cells is utilized as a polyvalent vaccine, whereas subsequent to administration antibody or t cell responses are assessed for induction of antigen specificity, peptides corresponding to immune response stimulated are used for further immunization to focus the immune response.

In some embodiments, culture of the immune effectors cells is performed after extracting from a patient that has been immunized with a polyvalent antigenic preparation. Specifically separating the cell population and cell sub-population containing a T cell can be performed, for example, by fractionation of a mononuclear cell fraction by density gradient centrifugation, or a separation means using the surface marker of the T cell as an index. Subsequently, isolation based on surface markers may be performed. Examples of the surface marker include CD3, CD8 and CD4, and separation methods depending on these surface markers are known in the art. For example, the step can be performed by mixing a carrier such as beads or a culturing container on which an anti-CD8 antibody has been immobilized, with a cell population containing a T cell, and recovering a CD8-positive T cell bound to the carrier. As the beads on which an anti-CD8 antibody has been immobilized, for example, CD8 MicroBeads), Dynabeads M450 CD8, and Eligix anti-CD8 mAb coated nickel particles can be suitably used. This is also the same as in implementation using CD4 as an index and, for example, CD4 MicroBeads, Dynabeads M-450 CD4 can also be used. In some embodiments of the invention, T regulatory cells are depleted before initiation of the culture. Depletion of T regulatory cells may be performed by negative selection by removing cells that express makers such as neuropilin, CD25, CD4, CTLA4, and membrane bound TGF-beta. Experimentation by one of skill in the art may be performed with different culture conditions in order to generate effector lymphocytes, or cytotoxic cells, that possess both maximal activity in terms of viral killing. For example, the step of culturing the cell population and cell sub-population containing a T cell can be performed by selecting suitable known culturing conditions depending on the cell population. In addition, in the step of stimulating the cell population, known proteins and chemical ingredients, etc., may be added to the medium to perform culturing. For example, cytokines, chemokines or other ingredients may be added to the medium. Herein, the cytokine is not particularly limited as far as it can act on the T cell, and examples thereof include IL-2, IFN-.gamma., transforming growth factor (TGF)-.beta., IL-15, IL-7, IFN-.alpha., IL-12, CD40L, and IL-27. From the viewpoint of enhancing cellular immunity, particularly suitably, IL-2, IFN-.gamma., or IL-12 is used and, from the viewpoint of improvement in survival of a transferred T cell in vivo, IL-7, IL-15 or IL-21 is suitably used. In addition, the chemokine is not particularly limited as far as it acts on the T cell and exhibits migration activity, and examples thereof include RANTES, CCL21, MIP1.alpha., MIP1.beta., CCL19, CXCL12, IP-10 and MIG. The stimulation of the cell population can be performed by the presence of a ligand for a molecule present on the surface of the T cell, for example, CD3, CD28, or CD44 and/or an antibody to the molecule. Further, the cell population can be stimulated by contacting with other lymphocytes such as antigen presenting cells (dendritic cell) presenting a target peptide such as a peptide derived from a cancer antigen on the surface of a cell. In addition to assessing cytotoxicity and migration as end points, it is within the scope of the current invention to optimize the cellular product based on other means of assessing T cell activity, for example, the function enhancement of the T cell in the method of the present invention can be assessed at a plurality of time points before and after each step using a cytokine assay, an antigen-specific cell assay (tetramer assay), a proliferation assay, a cytolytic cell assay, or an in vivo delayed hypersensitivity test using a recombinant viral-associated antigen or an immunogenic fragment or an antigen-derived peptide. Examples of an additional method for measuring an increase in an immune response include a delayed hypersensitivity test, flow cytometry using a peptide major histocompatibility gene complex tetramer. a lymphocyte proliferation assay, an enzyme-linked immunosorbent assay, an enzyme-linked immunospot assay, cytokine flow cytometry, a direct cytotoxicity assay, measurement of cytokine mRNA by a quantitative reverse transcriptase polymerase chain reaction, or an assay which is currently used for measuring a T cell response such as a limiting dilution method. In vivo assessment of the efficacy of the generated cells using the invention may be assessed in a living body before first administration of the T cell with enhanced function of the present invention, or at various time points after initiation of treatment, using an antigen-specific cell assay, a proliferation assay, a cytolytic cell assay, or an in vivo delayed hypersensitivity test using a recombinant viral-associated antigen or an immunogenic fragment or an antigen-derived peptide. Examples of an additional method for measuring an increase in an immune response include a delayed hypersensitivity test, flow cytometry using a peptide major histocompatibility gene complex tetramer. a lymphocyte proliferation assay, an enzyme-linked immunosorbent assay, an enzyme-linked immunospot assay, cytokine flow cytometry, a direct cytotoxicity assay, measurement of cytokine mRNA by a quantitative reverse transcriptase polymerase chain reaction, or an assay which is currently used for measuring a T cell response such as a limiting dilution method.

Within the context of the invention, teachings are provided to amplify an antigen specific immune response following immunization with a polyvalent vaccine, in which the antigenic epitopes are used for immunization together with adjuvants such as toll like receptors (TLRs). These molecules are type 1 membrane receptors that are expressed on hematopoietic and non-hematopoietic cells. At least 11 members have been identified in the TLR family. These receptors are characterized by their capacity to recognize pathogen-associated molecular patterns (PAMP) expressed by pathogenic organisms. It has been found that triggering of TLR elicits profound inflammatory responses through enhanced cytokine production, chemokine receptor expression (CCR2, CCR5 and CCR7), and costimulatory molecule expression. As such, these receptors in the innate immune systems exert control over the polarity of the ensuing acquired immune response. Among the TLRs, TLR9 has been extensively investigated for its functions in immune responses. Stimulation of the TLR9 receptor directs antigen-presenting cells (APCs) towards priming potent, T.sub.H1-dominated T-cell responses, by increasing the production of pro-inflammatory cytokines and the presentation of co-stimulatory molecules to T cells. CpG oligonucleotides, ligands for TLR9, were found to be a class of potent immunostimulatory factors

In some embodiments of the invention, specific antigens are immunized following polyvalent immunization, said specific antigens administered in the form of DNA vaccines. Numerous publications have reported animal and clinical efficacy of DNA vaccines which are incorporated by reference [28-30]. In addition to direct DNA injection techniques, DNA vaccines can be administered by electroporation [31]. The nucleic acid compositions, including the DNA vaccine compositions, may further comprise a pharmaceutically acceptable excipient. Examples of suitable pharmaceutically acceptable excipients for nucleic acid compositions, including DNA vaccine compositions, are well known to those skilled in the art and include sugars, etc. Such excipients may be aqueous or non aqueous solutions, suspensions, and emulsions. Examples of non-aqueous excipients include propylene glycol, polyethylene glycol, vegetable oils such as olive oil, and injectable organic esters such as ethyl oleate. Examples of aqueous excipient include water, alcoholic/aqueous solutions, emulsions or suspensions, including saline and buffered media. Suitable excipients also include agents that assist in cellular uptake of the polynucleotide molecule. Examples of such agents are (i) chemicals that modify cellular permeability, such as bupivacaine, (ii) liposomes or viral particles for encapsulation of the polynucleotide, or (iii) cationic lipids or silica, gold, or tungsten microparticles which associate themselves with the polynucleotides. Anionic and neutral liposomes are well-known in the art (see, e.g., Liposomes: A Practical Approach, RPC New Ed, IRL press (1990), for a detailed description of methods for making liposomes) and are useful for delivering a large range of products, including polynucleotides. Cationic lipids are also known in the art and are commonly used for gene delivery. Such lipids include Lipofectin™ also known as DOTMA (N—[I-(2,3-dioleyloxy) propyls N,N, N-trimethylammonium chloride), DOTAP (1,2-bis (oleyloxy)-3 (trimethylammonio) propane), DDAB (dimethyldioctadecyl-ammonium bromide), DOGS (dioctadecylamidologlycyl spermine) and cholesterol derivatives such as DCChol (3 beta-(N—(N′,N′-dimethyl aminomethane)-carbamoyl) cholesterol). A description of these cationic lipids can be found in EP 187,702, WO 90/11092, U.S. Pat. No. 5,283,185, WO 91/15501, WO 95/26356, and U.S. Pat. No. 5,527,928. A particular useful cationic lipid formulation that may be used with the nucleic vaccine provided by the disclosure is VAXFECTIN, which is a commixture of a cationic lipid (GAP-DMORIE) and a neutral phospholipid (DPyPE) which, when combined in an aqueous vehicle, self-assemble to form liposomes. Cationic lipids for gene delivery are preferably used in association with a neutral lipid such as DOPE (dioleyl phosphatidylethanolamine), as described in WO 90/11092 as an example. In addition, a DNA vaccine can also be formulated with a nonionic block copolymer such as CRL1005. Other immunization means include prime boost regiments [32]. The polypeptide and nucleic acid compositions can be administered to an animal, including human, by a number of methods known in the art. Examples of suitable methods include: (1) intramuscular, intradermal, intraepidermal, intravenous, intraarterial, subcutaneous, or intraperitoneal administration, (2) oral administration, and (3) topical application (such as ocular, intranasal, and intravaginal application). One particular method of intradermal or intraepidermal administration of a nucleic acid vaccine composition that may be used is gene gun delivery using the Particle Mediated Epidermal Delivery (PMED™) vaccine delivery device marketed by PowderMed [33]. PMED is a needle-free method of administering vaccines to animals or humans. The PMED system involves the precipitation of DNA onto microscopic gold particles that are then propelled by helium gas into the epidermis [34]. The DNA-coated gold particles are delivered to the APCs and keratinocytes of the epidermis, and once inside the nuclei of these cells, the DNA elutes off the gold and becomes transcriptionally active, producing encoded protein. This protein is then presented by the APCs to the lymphocytes to induce a T-cell-mediated immune response. Another particular method for intramuscular administration of a nucleic acid vaccine provided by the present disclosure is electroporation [35]. Electroporation uses controlled electrical pulses to create temporary pores in the cell membrane, which facilitates cellular uptake of the nucleic acid vaccine injected into the muscle [36-39]. Where a CpG is used in combination with a nucleic acid vaccine, it is preferred that the CpG and nucleic acid vaccine are co-formulated in one formulation and the formulation is administered intramuscularly by electroporation. A helper T cell and cytotoxic T cell stimulatory polypeptide can be introduced into a mammalian host, including humans, linked to its own carrier or as a homopolymer or heteropolymer of active polypeptide units. Such a polymer can elicit increase immunological reaction and, where different polypeptides are used to make up the polymer, the additional ability to induce antibodies and/or T cells that react with different antigenic determinants of the virus. Useful carriers known in the art include, for example, thyroglobulin, albumins such as human serum albumin, tetanus toxoid, polyamino acids such as poly(D-lysine:D-glutamic acid), influenza polypeptide, and the like. Adjuvants such as incomplete Freunds adjuvant, GM-CSF, aluminum phosphate, CpG containing DNA, inulin, Poly (IC), aluminum hydroxide, alum, or montanide can also be used in the administration of an helper T cell and cytotoxic T cell stimulatory polypeptide.

Subsequent to augmentation of lymphocyte numbers specific for killing of the virus, modification of the microenvironment may be performed. In one embodiment, macrophage modulators are used.

Macrophages are key components of the innate immune system which play a principal role in the regulation of inflammation as well as physiological processes such as tissue remodeling [40, 41]. The diverse role of macrophages can be seen in conditions ranging from wound healing [42-45], to myocardial infarction [46-52], to renal failure [53-56] and liver failure [57].

Differentiated macrophages and their precursors are versatile cells that can adapt to microenvironmental signals by altering their phenotype and function [58]. Although they have been studied for many years, it has only recently been shown that these cells comprise distinct sub-populations, known as classical M1 and alternative M2 [59]. Mirroring the nomenclature of Th1 cells, M1 macrophages are described as the pro-inflammatory sub-type of macrophages induced by IFN-.gamma. and LPS. They produce effector molecules (e.g., reactive oxygen species) and pro-inflammatory cytokines (e.g., IL-12, TNF-.alpha. and IL-6) and they trigger Th1 polarized responses [60].

Macrophages can play a virus inhibitory, as well as a virus stimulatory role. Initial studies supported the role of macrophages in mediating antibody dependent cellular cytotoxicity in virus's [61-68], and thus being associated with potentiation of antivirus immune responses. Macrophages also possess the ability to directly recognize virus by virtue of virus expressed “eat-me” signals, which include the stress associated protein calreticulin [69, 70], which binds to the low-density lipoprotein receptor-related protein (LRP) on macrophages to induce phagocytosis [71]. Virus's protect themselves by expression of CD47, which binds to macrophage SIRP-1 and transduces an inhibitory signal [72]. Blockade of CD47 using antibodies results in remission of cancers mediated by macrophage activation [73-77]. Thus on the one hand, macrophages play an important role in induction of antivirus immunity. This can also be exemplified by some studies, involving administration of GM-CSF in order to augment macrophage numbers and activity in cancer patients [78-81].

Unfortunately, there is also evidence that macrophages support virus growth. Studies in the osteopetrotic mice strain, which lacks mature macrophages, demonstrate that virus actually grow slower in animals deficient in macrophages [82]. Several other animal models have elegantly demonstrated that macrophages contribute to virus growth, in part through stimulating on the angiogenic switch [83-85]. Numerous virus biopsy studies have shown that there is a negative correlation between macrophage infiltration and patient survival [86-90].

The importance of macrophages in clinical implementation of cancer therapeutics can be seen from results of a double blind clinical trials in metastatic colorectal cancer patients where cetuximab (anti-epidermal growth factor receptor (EGFR) monoclonal antibody (mAb)) was added to a protocol comprising of bevacizumab and chemotherapy. The addition of cetuximab actually resulted in decreased survival. In a study examining whether monocyte conversion to M2 angiogenic macrophages was responsible, investigators observed that CD163-positive M2 macrophages where found in high concentrations intravirusally in patients with colorectal carcinomas. These M2 cells expressed abundant levels of Fc-gamma receptors (FcγR) and PD-L1. Additionally, consistent with the M2 phenotype the cells generated large amounts of the immunosuppressive molecule IL-10 and the angiogenic mediator VEGF. When M2 cells were cultured with EGFR-positive virus cells loaded with low concentrations of cetuximab, further augmentation of IL-10 and VEGF production was observed. These data suggest that under certain contexts, virus manipulate macrophages to take on the M2 phenotype, and this subsequently leads to enhanced virus progressing factors when virus cells are bound by antibodies [91].

Manipulation of macrophages to inhibit M2 and shift to M1 phenotype may be performed using a variety of means. One theme that seems unifying is the ability of toll like receptor (TLR) agonists to influence this. In addition to cytokine differences, macrophages capable of killing virus cells are usually known to express low levels of the inhibitory Fc gamma receptor IIb, whereas virus promoting macrophages have high levels of this receptor [92]. Furthermore, virus associated cytokines such as IL-4 and IL-10 are known to induce upregulation of the Fc gamma receptor IIB [93-96].

In one study, the effect of the TLR7/8 agonist R-848 was assessed on monocytes derived from human peripheral blood. It was found that 12 hour exposure of R-848 increased FcgammaR-mediated cytokine production and antibody-dependent cellular cytotoxicity by monocytes. Furthermore, upregulation of the ADCC associated receptors FcgammaRI, FcgammaRIIa, and the common gamma-subunit was observed. However treatment with R-848 led to profound downregulation of the inhibitory FcgammaRIIb molecule [97]. These data support ability to modify therapeutic activity of macrophages by manipulation of TLR signaling pathways. Other TLRs have been found to suppress inhibitory receptors on macrophages. For example, in another study it was observed that exposing monocytes to TLR4 agonists leads to suppression of the FcγRIIb macrophage inhibitory protein by MARCH3 mediated ubiquitination [98].

In one embodiment administration of ImmunoMax is performed systemically, and/or locally, which is an injectable polysaccharide purified from potato sprouts and approved as pharmaceutical in the Russian Federation (registration P No.001919/02-2002) and 5 other countries of Commonwealth of Independent States (formerly the USSR) and has been evaluated in a wide range of medical situations. In accordance with the formal “Instruction of Medical Use”, one medical indication for Immunomax® is the stimulation of immune defense during the treatment of different infectious diseases (http://www.gepon.ru/immax_intro.htm). Studies have shown that Immunomax® induces immune mediated killing of virally infected cells in a TLR4 dependent manner [99]. In one embodiment of the invention, ImmunoMax is utilized to induce an M2 to M1 shift, thus reducing macrophage derived immune suppressants and augmenting production of immune stimulatory cytokines such as IL-12 and TNF-alpha [99]. In some embodiments of the invention, other agents may be used to modulate M2 to M1 transition of virus associated macrophages including RRx-001 [100], the bee venom derived peptide melittin [101], CpG DNA [102, 103], metformin [104], Chinese medicine derivative puerarin [105], rhubarb derivative emodin [106], dietary supplement chlorogenic acid [107], propranolol [108], poly ICLC [109], BCG [110], Agaricus blazei Murill mushroom extract [111], endotoxin [112], olive skin derivative maslinic acid [113], intravenous immunoglobulin [114], phosphotidylserine targeting antibodies [115], dimethyl sulfoxide [116],surfactant protein A [117], Zoledronic acid [118], bacteriophages [119],

Prior to induction of immunogenic cell death, antigen presenting cells are administered within the current invention, one of the most potent antigen presenting cells is the dendritic cell. Dendritic cells (DC) possess unique morphology similar to neuronal dendrites and were originally identified based on their ability to stimulate the adaptive immune system. Of importance to the field of virus immunotherapy, dendritic cells appear to be the only cell in the body capable of activating naïve T cells [120]. The concept of dendritic cells instructing naïve T cells to differentiate into effector or memory cells is fundamental because it places the dendritic cell as the most powerful antigen presenting cell. This implies that for immunotherapeutic purposes dendritic cells do not necessarily need to be administered at high numbers in patients. One way in which dendritic cells have been described is as sentinels of the immune system that are patrolling the body in an immature state [121, 122]. Once DC are activated, by a stimulatory signal such as a Damage Associated Molecular Patterns (DAMPS) the DC then migrate into the draining lymph nodes through the afferent lymphatics. During the trafficking process, DC degrade ingested proteins into peptides that bind to both MHC class I molecules and MHC class II molecules. This allows the DC to: a) perform cross presentation in that they ingest exogenous antigens but present peptides in the MHC I pathway; and b) activate both CD8 (via MHC I) and CD4 (via MHC II). Interestingly, lipid antigens are processed via different pathways and are loaded onto non-classical MHC molecules of the CD1 family [123].

Generation of clinical grade dendritic cells is known in the art. For references, one of skill in the art is referred to the following clinical trial in melanoma [124-175], soft tissue sarcoma [176], thyroid [177-179], glioma [180-201], multiple myeloma [202-210], lymphoma [211-213], leukemia [214-221], as well as liver [222-227], lung [228-241], ovarian [242-245], and pancreatic cancer [246-248].

T cell modulator (TCM) is a pharmaceutical grade transfer factor, which activates T cells by reducing costimulatory requirements, thus potentially increasing infiltration of tumors by T cells. The concept of an immunologically acting “Transfer Factor” was originally identified by Henry Lawrence in a 1956 publication [276], in which he reported simultaneous transfer of delayed hypersensitivity to diphtheria toxin and to tuberculin in eight consecutive healthy volunteers who received extracts from washed leucocytes taken from the peripheral blood of tuberculin-positive, Schick-negative donors who were highly sensitive to purified diphtheria toxin and toxoid. The leucocyte extracts used for transfer contained no detectable antitoxin. The recipient subjects were Schick-positive (<0.001 unit antitoxin per ml. serum) and tuberculin-negative at the time of transfer. All the recipients remained Schick-positive for at least 2 weeks following transfer and in every case their serum contained less than 0.001 units antitoxin at the time when they exhibited maximal skin reactivity to toxoid. The “transfer factor” that was utilized was prepared by washing packed leukocytes isolated using the bovine fibrinogen method, and washing the leukocytes twice in recipient plasma. The washed leukocytes were subsequently lysed by 7-10 freeze-thaw cycles in the presence of DNAse with Mg++. Administration of the extract was performed intradermally and subcutaneously over the deltoid area.

Given that in those early days little was known regarding T cell specificity and MHC antigen presentation, the possibility that immunological information was transmitted by these low molecular weight transfer factors was taken seriously. Transfer factors of various sizes and charges were isolated, with some concept that different antigens elicited different types of transfer factors [277, 278]. Numerous theories were proposed to the molecular nature of transfer factor. Some evidence was that it constituted chains of antibodies that were preformed but subsequently cleaved [279]. Functionally, one of the main thoughts was that transfer factor has multiple sites of action, including effects on the thymus, on lymphocyte-monocyte and/or lymphocyte-lymphocyte interactions, as well as direct effects on cells in inflammatory sites. It is also suggested that the “specificity” of transfer factor is determined by the immunologic status of the recipient rather than by informational molecules in the dialysates [280]. Burger et al [281], used exclusion chromatography to perform characterization of transfer factor. The found that specific transferring ability of transfer factor in vivo was found to reside in the major UV-absorbing peak (Fraction III). Fraction III transferred tuberculin, candida, or KLH-reactivity to previously negative recipients. Fraction III from nonreactive donors was ineffective. When the fractions were tested in vitro, we found that both the mitogenic activity of whole transfer factor and the suppressive activity to mitogen activation when present in transfer factor was found in Fraction I. Fraction III contained components responsible for augmentation of PHA and PWM responses. In addition, Fraction III contained the component responsible for antigen-dependent augmentation of lymphocyte transformation. Fraction IV was suppressive to antigen-induced lymphocyte transformation.

In 1992 Kirkpatrick characterized the specific transfer factor at molecular level. The transfer factor is constituted by a group of numerous molecules, of low molecular weight, from 1.0 to 6.0 kDa. The 5 kDa fraction corresponds to the transfer factor specific to antigens. There are a number of publications about the clinical indications of the transfer factor for diverse diseases, in particular those where the cellular immune response is compromised or in those where there is a deficient regulation of the immune response. It has been demonstrated that the transfer factor increases the expression of IFN-gamma and RANTES, while decreases the expression of osteopontine. Using animal models it has been reported that transfer factor possesses activity against M. tuberculosis, and with a model of glioma with good therapeutic results. In the clinical setting studies have reported effects against herpes zoster, herpes simplex type I, herpetic keratitis, atopic dermatitis, osteosarcoma, tuberculosis, asthma, post-herpetic neuritis, anergic coccidioidomycosis, leishmaniasis, toxoplasmosis, mucocutaneous candidiasis, pediatric infections produced by diverse pathogen germs, sinusitis, pharyngitis, and otits media. All of these diseases were studied through protocols which main goals were to study the therapeutic effects of the transfer factor, and to establish in a systematic way diverse dosage schema and time for treatment to guide the prescription of the transfer factor [282].

In some embodiments of the invention, administration of intravenous vitamin C is utilized. Patients treated with immunotherapy have been shown to develop a scurvy-like condition. The patient presented with acute signs and symptoms of scurvy (perifollicular petechiae, erythema, gingivitis and bleeding). Serum ascorbate levels were significantly reduced to almost undetectable levels [269]. Although the role of ascorbic acid (AA) hypersupplementation in stimulation of immunity in healthy subjects is controversial, it is well established that AA deficiency is associated with impaired cell mediated immunity. This has been demonstrated in numerous studies showing deficiency suppresses T cytotoxic responses, delayed type hypersensitivity, and bacterial clearance [270]. Additionally, it is well-known that NK activity, which IL-2 is anti-tumor activity is highly dependent on, is suppressed during conditions of AA deficiency [271]. Thus it may be that while IL-2 therapy on the one hand is stimulating T and NK function, the systemic inflammatory syndrome-like effects of this treatment may actually be suppressed by induction of a negative feedback loop. Such a negative feedback loop with IL-2 therapy was successfully overcome by work using low dose histamine to inhibit IL-2 mediated immune suppression, which led to the “drug” Ceplene (histamine dichloride) receiving approval as an IL-2 adjuvant for treatment of AML [272].

The concept of AA deficiency subsequent to IL-2 therapy was reported previously by another group. Marcus et al evaluated 11 advanced cancer patients suffering from melanoma, renal cell carcinoma and colon cancer being on a 3 phase immunotherapeutic program consisting of: a) 5 days of i.v. high-dose (10(5) units/kg every 8 h) interleukin 2, (b) 6½ days of rest plus leukapheresis; and (c) 4 days of high-dose interleukin 2 plus three infusions of autologous lymphokine-activated killer cells. Mean plasma ascorbic acid levels were normal (0.64+/−0.25 mg/dl) before therapy. Mean levels dropped by 80% after the first phase of treatment with high-dose interleukin 2 alone (0.13+/−0.08 mg/dl). Subsequently plasma ascorbic acid levels remained severely depleted (0.08 to 0.13 mg/dl) throughout the remainder of the treatment, becoming undetectable (less than 0.05 mg/dl) in eight of 11 patients during this time. Importantly, blood pantothenate and plasma vitamin E remained within normal limits in all 11 patients throughout the phases of therapy, suggesting the hypovitaminosis was specific AA. Strikingly, Responders (n=3) differed from nonresponders (n=8) in that plasma ascorbate levels in the former recovered to at least 0.1 mg/dl (frank clinical scurvy) during Phases 2 and 3, whereas levels in the latter fell below this level [273]. Similar results were reported in another study by the same group examining an additional 15 patients [274]. The possibility that prognosis was related to AA levels is intriguing because of the possibility of higher immune response in these patients, however this has not been tested.

The state of AA deficiency in cancer patients, whether or not as a result of inflammation, suggests supplementation may yield benefit in quality of life. Indeed this was one of the main findings that stimulating us to write this review [275]. Improvements in quality of life were also noted in the early studies of Murata et al [276], as well as Cameron [277]. But in addition to this endpoint there appears to be a growing number of studies suggesting direct anti-cancer effects via generation of free radicals locally at tumor sites [278]. In vitro studies on a variety of cancer cells including neuroblastoma [279], bladder cancer [280], pancreatic cancer [281], mesothelioma [282], hepatoma [283], have demonstrated cytotoxic effects at pharmacologically achievable concentrations.

Enhancement of cytotoxicity of Docetaxel, Epirubicin, Irinotecan and 5-FU to a battery of tumor cell lines by AA was demonstrated in vitro [284]. In vivo studies have also supported the potential anticancer effects of AA. For example, Pollard et al used the rat PAIII androgen-independent syngeneic prostate cancer cell line to induce tumors in Lobund-Wistar rats. Daily intraperitoneal administration of AA for 30 days, with evaluation at day 40 revealed significant inhibition of tumor growth, as well as reduction in pulmonary and lymphatic metastasis [285]. Levine's group reported successful in vivo inhibition of human xenografted glioma, overian, and neuroblastoma cells in immune deficient animals by administration of AA. Interestingly control fibroblasts were not affected [286]. Clinical reports of remission induced by IV AA have been published [287], however, as mentioned above, formal trials are still ongoing.

In addition to direct cytotoxicity of AA on tumor cells, inhibition of angiogenesis may be another mechanism of action. It has been reported that AA inhibits HUVEC proliferation in vitro [288], as well as suppressing neovascularization in the chorionic allontoic membrane assay [289]. Recently we have reported that in vivo administration of AA results in suppressed vascular cord formation in mouse models [290]. Supporting this possibility, Yeom et al demonstrated that parenteral administration of AA in the S-180 sarcoma model leads to reduced tumor growth, which was associated with suppression of angiogenesis and the pro-angiogenic factors bFGF, VEGF, and MN/IP-2 [291]. Recent studies suggest that AA suppresses activation of the hypoxia inducible factor (HIF)-1, which is a critical transcription factor that stimulates tumor angiogenesis [292-294]. The clinical relevance of this has been demonstrated in a study showing that endometrial cancer patients having reduced tumor ascorbate levels possess higher levels of HIF-1 activation and a more aggressive phenotype [295].

Thus the possibility exists that administration of AA for treatment of tumor inflammatory mediated pathologies may also cause an antitumor effect. Whether this effect is mediated by direct tumor cytotoxicity or inhibition of angiogenesis remains to be determined. Unfortunately none of the ongoing trials of AA in cancer patients seek to address this issue [296-301].

Despite numerous claims in the popular media and even on vitamin labels, the concept of AA stimulating immunity is not as clear-cut. Part of this is because ROS are involved in numerous signals of immune cells [302]. For example, it is known that T cell receptor signaling induces an intracellular flux of ROS which is necessary for T cell activation [303]. There are numerous studies demonstrating ascorbic acid under certain conditions actually can inhibit immunity. For example, high dose ascorbate inhibits T cell and B cell proliferative responses as well as IL-2 secretion in vitro [304, 305], as well as NK cytotoxic activity [306]. Additionally, AA has been demonstrated to inhibit T cell activating ability of dendritic cells by rendering them in an immature state in part through inhibition of NF-kappa B [307].

However, It appears that the immune stimulatory effects of AA are actually observed in the context of background immune suppression or in situations of AA deficiency, both of which are well-known in the cancer and SIRS patient. A common occurrence in cancer [308-312] and SIRS patients [313, 314] is the presence of a cleaved T cell receptor (TCR) zeta chain. The zeta chain is an important component of T cell and NK cell activation, that bears the highest number of immunoreceptor tyrosine-based activation motifs (ITAMs) of other TCR and NK signaling molecules [315]. At a cellular level cleavage of the zeta chain is associated with loss of T/NK cell function and spontaneous apoptosis [316-318], at a clinical level it is associated with poor prognosis [319-324].

Since loss of TCR zeta chain is found in other inflammatory conditions ranging from hemodialysis [325, 326], to autoimmunity [327-330], to heart disease [331], the possibility that inflammatory mediators such as ROS cause TCR zeta downregulation has been suggested. Circumstantial evidence comes from studies associated inflammatory cells such as tumor associated macrophages (TAMS) with suppression of zeta chain expression [332]. Myeloid suppressor cells, which are known to produce high concentrations of ROS [333-335] have also been demonstrated to induce reduction of TCR zeta chain in cancer [336], and post trauma [337]. Administration of anti-oxidants has been shown to reverse TCR zeta chain cleavage in tissue culture [338, 339]. Therefore, from the T cell side of immunity, an argument could be made that intravenous ascorbic acid may upregulate immunity by blocking zeta chain downregulation in the context of cancer and acute inflammation.

While it is known that AA functions as an antioxidant in numerous biological conditions, as well as reduces inflammatory markers, the possibility that AA actually increases immune function in cancer patients, as well as is effects on survival and other cancer-related events, has never been formally tested. IV AA has a long and controversial history in relation to reducing tumors in patients. This has impeded research into other potential benefits of this therapy in cancer patients such as reduction of inflammation, improvement of quality of life, and impeding SIRS initiation and progression to MOF. While ongoing clinical trials of IV AA for cancer may or may not meet the bar to grant this modality a place amongst the recognized chemotherapeutic agents, it is critical that we collect as much biological data as possible, given the possibility of this agent to be a meaningful adjuvant therapy.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

### Examples

Materials and Methods Methods Cell Lines

HeLa human cervical cancer cells were obtained from American Type Tissue Culture (ATCC: Manassas, Va.) and grown under fully humidified 5% CO2 environment with MEM supplemented with 10% FBS, 2% sodium pyruvate, non-essential amino acids (2 mM), penicillin (100 units/ml), streptomycin (100 μg/ml), and glutamine (4 mM) (Gibco-BRL). Cells were passaged by trypsinization twice weekly or as needed based on 75% confluency.

Peripheral Blood Mononuclear Cells (PBMC)

PBMC were isolated from buffy coats by density-gradient centrifugation. Specifically, buffy coat cells were dispensed over five 50 ml falcon tubes, phosphate-buffered saline (PBS)/2% fetal calf serum (FCS) solution was added to reach a volume of 20 ml and 10 ml Ficoll-Paque® was gently added under the diluted buffy coat cells. Centrifugation was performed at 400 g for 20 min at room temperature (RT) and washing of PBMC was done three times with PBS/2% FCS. Culture of freshly isolated PBMC was performed in complete MEM media.

Cell Treatments and Analysis

ACTIVEIMMUNE™ is a commercial transfer factor made as follows. Buffy coat leukocytes, isolated from centrifugation of coagulated peripheral blood or splenocytes, is concentrated to 2×10⁽⁸⁾ cells per ml in saline. The concentrated leukocytes or splenocytes are then subjected to 7 freeze-thaw cycles between −70 Celsius and 37 Celsius. Subsequent to freeze-thawing, the resultant substance is dialyzed for 24 hours utilizing an excess of sterile water over a peristaltic pump. The dialysate is then lyophilized in order to achieve concentration. Said concentrate is then ultrafiltered through a 10 kDa filter and heated to 60 Celsius. The material is subsequently filtered through a 2 micron filter, and lyophilized. ACTIVEIMMUNE™ was diluted in complete MEM media prepared as described above. Dilutions of 1:10, 1:100, 1:1000 and 1:10,000 were performed. Negative controls were complete MEM media. Positive controls were concanavalin A at a concentration of 2.5 ug/ml. PBMC were plated at 1.5×10⁶ cells/ml in flat-bottom 96-well culture plates in a volume of 200 μl per well and incubated at 37° in a humidified 5% CO2 atmosphere. Conditioned media was then evaluated for IFN-gamma production using ELISA from R & D Systems (Quantikine ELISA). Concentration was calculated by plotting against a standard curve generated with control cytokine.

HeLa cells were plated at a concentration of 10,000 cells per well in flat bottom plates and incubated with dilutions of ACTIVEIMMUNE™ at 1:10, 1:100, 1:1000 and 1:10,000. The 3-(4,5-dimethylthiazol-2-yl)-2,5-diphenyl tetrazolium bromide (MTT) assay was performed for assessment of proliferation. In this assay soluble MTT is metabolized by mitochondrial enzyme activity of viable tumor cells, into an insoluble colored formazan product. Subsequently formazan were dissolved in DMSO and measured spectrophotometrically at 540 nm. Briefly, 200 μl of cell suspension was seeded in 96-well microplates and incubated for 48 h (37° C., 5% CO2 air humidified).

To evaluate cell survival, 20 μl of MTT solution (5 mg/ml in PBS) was added to each well and incubated for 3 h. Then gently 150 μl of old medium containing MTT was replaced by DMSO and pipetted to dissolve any formed formazan crystals. Absorbance was then determined at 540 nm by enzyme-linked immunosorbent assay (ELISA) plate reader. Each extract concentration was assayed in 4 wells and repeated 3-times.

ELISA

IFN-gamma, IL-4, IL-10 and IL-12 were assessed by ELISA (R and D Systems) utilizing supernatant from mitogen activated cultures and treated DC.

Dendritic Cells

DC were generated from PBMC resuspended in RPMI-10% FCS, and allowed to adhere to 6-well plates (Costar Corp., Cambridge, Mass.). After 2 h incubation at 37 Celsius, the nonadherent cells were removed and the adherent cells washed in phosphate buffered saline (PBS), followed by detachment by incubation with Mg 2+ and Ca 2+ free PBS containing 0.5 mM EDTA at 37 Celsius. The adherent fraction was subsequently cultured at 3×10(6)/ml in RPMI-10% FCS supplemented with 50 ng/ml GM-CSF and 1,000 U/ml IL-4. Media is changed every 2 days for a total of 8 days culture. DC were isolated by positive selection for CD83 and subsequently treated with ACTIVEIMMUNE™ on day 6 of culture. Assessment of maturation was performed by flow cytometry for CD80 and CD86 expression.

Blockade of TLR-4 was performed using by culture in the presence of TLR4 antagonist LPS-RS (Invivogen (San Diego, Calif.), (5 μg/mL), with pretreatment 4 hours before exposure to ACTIVEIMMUNE™.

Results

ACTIVEIMMUNE™ Does Not Modulate Cellular Proliferation

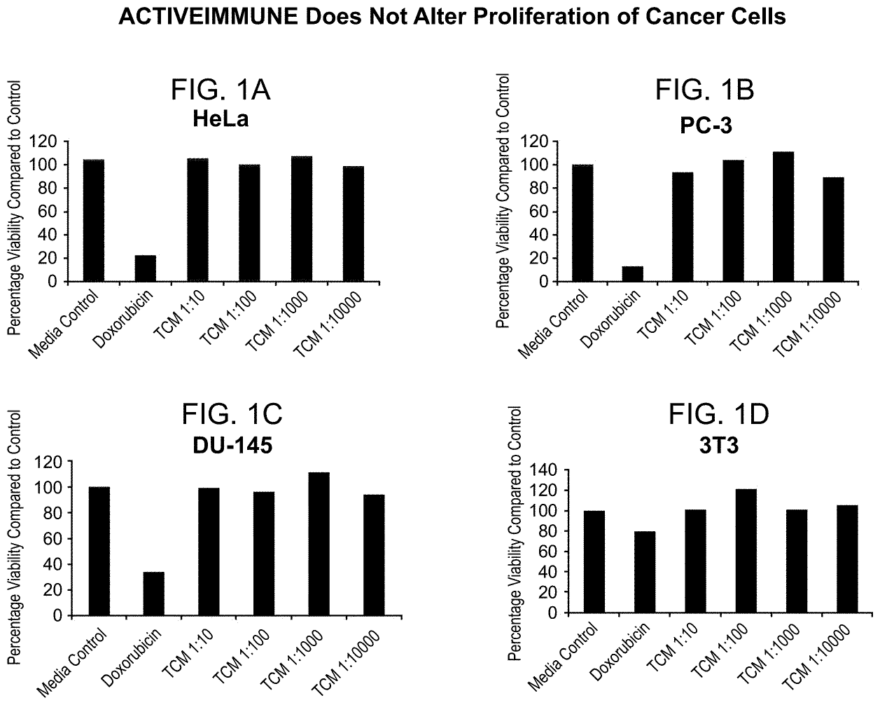

ACTIVEIMMUNE™ has been reported to possess anticancer activity. Accordingly, we conducted a series of experiments assessing ability of various concentrations of ACTIVEIMMUNE™ to inhibit proliferation of HeLa cells. We utilized the chemotherapeutic drug doxorubicin as a control. As seen in FIG. 1A, various doses of ACTIVEIMMUNE™ did not affect proliferation of HeLa cells as assessed in the MTT assay after 48 hours of culture. Importantly, supraphysiological doses of ACTIVEIMMUNE™, as high as 1:10 diluted volume by volume in the tissue culture media did not result in inhibition of proliferation. These data suggest that ACTIVEIMMUNE™ does not act through cytotoxic or cytostatic mechanisms. These data were confirmed with other cell lines such as PC-3, DU-145, and non-malignant 3T3 fibroblasts (See FIGS. 1B-1D).

ACTIVEIMMUNE™ Acts as a Cofactor for Cytokine Secretion from Immune Cells found in Peripheral Blood

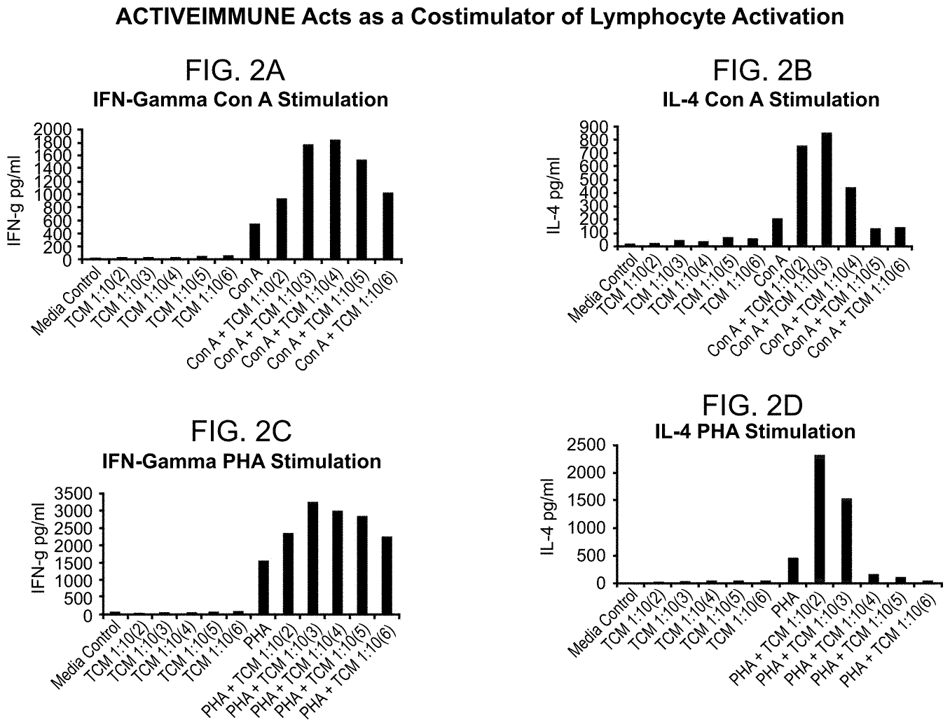

To assess whether ACTIVEIMMUNE™ directly activates T cell production of cytokines, or whether it requires a costimulatory signal, such as concanavalin A (ConA), was examined. ACTIVEIMMUNE™ did not affect viability of PBMC (data not shown). It appears that ACTIVEIMMUNE™ complements existing production of immune stimulatory molecules after a primary stimuli, but does not initiate immunity, at least based on IFN-gamma and IL-4 production (FIGS. 2a and 2b). Given that different doses of ACTIVEIMMUNE™ possess different costimulatory profiles for the different cytokines, we questioned whether the effect was specific to conconavalin A stimulation, or whether other factors may be at play. Accordingly, we substitute stimulation by conconavalin A to stimulation by phytohemagglutinin, a mitogen often used in studies stimulating human T cells. As seen in FIGS. 2c and 2d, a similar pattern of IFN-gamma and IL-4 costimulation was observed with PHA acting as the primary stimulator.

ACTIVEIMMUNE′ Induces Dendritic Cell Maturation in a TLR4 Dependent Manner

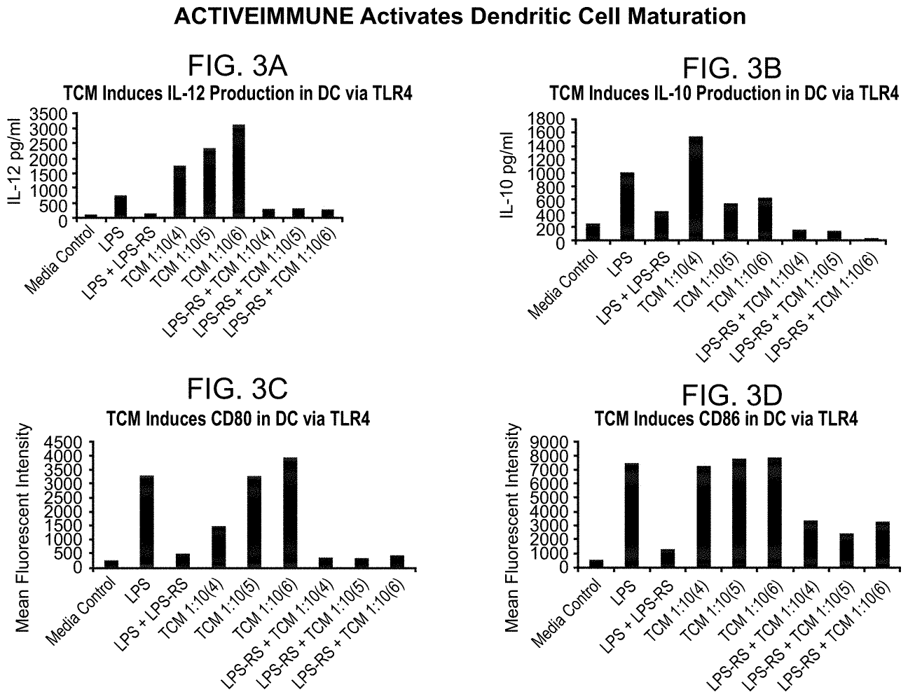

Given the contamination of antigen presenting cells in PBMC, and the fact that antigen presenting cells may be sending costimulatory signals to the T cells in response to ACTIVEIMMUNE′ treatment, a series of experiments were conducted to assess whether ACTIVEIMMUNE′ acts on the most potent antigen presenting cell, the dendritic cell. Day 6 immature DC generated from monocytes by IL4 and GM-CSF treatment were used to assess maturation-inducing potential of ACTIVEIMMUNE™. Cells were treated with saline, lps positive control, and 3 concentrations of ACTIVEIMMUNE™. Additionally, blockade of TLR4 signaling was performed by cotreatment with LPS-RS, an antagonist of the TLR-4 receptor. As seen in FIGS. 3a and 3b, ACTIVEIMMUNE′ was capable of upregulating expression of IL-12 and IL-10, respectively, suggesting from a functional perspective that DC activation was occurring. Indeed the fact that IL-12 drives Th1 cytokine production and IL-10 drives Th2, these data are in agreement with the previous data suggesting that ACTIVEIMMUNE′ is capable of modulating immunity. Definitive evidence of maturation of DC was observed using flow cytometry, demonstrating that upregulation of CD80 and CD86 was occurring as a result of ACTIVEIMMUNE′ treatment (FIGS. 3c and 3d). In all experiments, blockade of TLR-4 by treatment with LPS-RS, an antagonist of TLR4, resulted in marked reduction of both LPS induced changes (positive control) as well as in activity of ACTIVEIMMUNE™.

### Molecular Characterization of ACTIVEIMMUNE™

### Two Dimensional Gel Electrophoresis

Two-dimensional electrophoresis was performed according to the carrier ampholine method of isoelectric focusing (O'Farrell, P. H., J. Biol. Chem. 250: 4007-4021, 1975, Burgess-Cassler, A., Johansen, J., Santek, D., Ide J., and Kendrick N., Clin. Chem. 35: 2297, 1989) by Kendrick Labs, Inc. (Madison, Wis.) as follows: Isoelectric focusing was carried out in a glass tube of inner diameter 2.3 mm using 2% pH 3-10 isodalt Servalytes (Serva, Heidelberg, Germany) for 9600 volt-hrs. One μg of an IEF internal standard, tropomyosin, was added to the sample. This protein migrates as a doublet with lower polypeptide spot of MW 33,000 and pI 5.2. The enclosed tube gel pH gradient plot for this set of Servalytes was determined with a surface pH electrode.

For the 10% acrylamide gels, after equilibration for 10 min in Buffer ‘0’ (10% glycerol, 50 mM dithiothreitol, 2.3% SDS and 0.0625 M tris, pH 6.8), each tube gel was sealed to the top of a stacking gel that overlaid a 10% acrylamide slab gel (0.75 mm thick). SDS slab gel electrophoresis was carried out for about 4 hrs at 15 mA/gel. The following proteins (Sigma Chemical Co., St. Louis, Mo. and EMD Millipore, Billerica, Mass.) were used as molecular weight standards: myosin (220,000), phosphorylase A (94,000), catalase (60,000), actin (43,000), carbonic anhydrase (29,000) and lysozyme (14,000). These standards appear along the basic edge of the silver-stained(Oakley, B. R., Kirsch, D. R. and Moris, N. R. Anal. Biochem. 105:361-363, 1980) 10% acrylamide slab gel. The gel was dried between sheets of cellophane with the acid edge to the left.

After equilibration for 15 min in Buffer “0” (10% glycerol, 50 mM dithiothreitol, 2.3% SDS and 0.0625 M tris, pH 6.8) each tube gel was sealed to the top of 10% acrylamide spacer gels which are on the top of 16.5% acrylamide peptide slab gels (Shagger, H. and Jagow, G. Anal. Biochem. 166: 368, 1987) (0.75 mm thick). SDS slab gel electrophoresis was started at 15 mamp/gel for the first four hours and then carried out overnight at 12 mamp/gel as for the separation of peptides. The slab gel electrophoresis was stopped after the bromophenol blue dye front had just started running off the gel. The following proteins (Sigma Chemical Co., St. Louis, Mo. and EMD Millipore, Billerica, Mass.) were added as molecular weight markers: phosphorylase A (94,000), catalase (60,000), actin (43,000) and lysozyme (14,000). These standards appear as bands on the basic edge of the silver stained (Oakley, B. R., Kirsch, D. R. and Moris, N. R. Anal. Biochem. 105:361-363, 1980) 16.5% acrylamide slab gel. Low molecular weight markers from Sigma Chemical were also loaded myoglobin (polypetide backbone) 1-153 16,950; Myoglobin (I+II, 1-131) 14,440; myoglobin (I+III, 56-153) 10,600; Myoglobin (I, 56-131) 8,160; myoglobin (II 1-55) 6,210; Glucagon 3,480; and Myoglobin (III, 132-153) 2,510. The gel was silver-stained and dried between sheets of cellophane paper with the acid edge to the left.

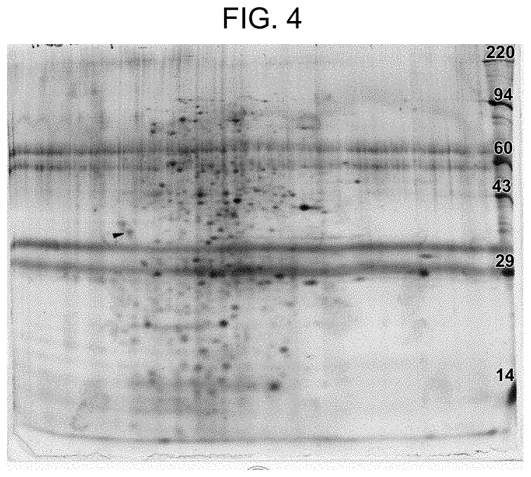

FIG. 4 illustrates the gel run under 10% conditions The arrowhead illustrates the molecular weight spot indicative of the immune modulatory activity that was subsequently sequenced.

Proteomic Analysis/Sequencing

Protein digestion and peptide extraction. Proteins that were separated by SDS-PAGE/2D-PAGE and stained by Coomassie dye were excised, washed and the proteins from the gel were treated according to published protocols [157-159]. Briefly, the gel pieces were washed in high purity, high performance liquid chromatography HPLC grade water, dehydrated and cut into small pieces and destained by incubating in 50 mM ammonium bicarbonate, 50 mM ammonium bicarbonate/50% acetonitrile, and 100% acetonitrile under moderate shaking, followed by drying in a speed-vac concentrator. The gel bands were then rehydrated with 50 mM ammonium bicarbonate. The procedure was repeated twice. The gel bands were then rehydrated in 50 mM ammonium bicarbonate containing 10 mM DTT and incubated at 56° C. for 45 minutes. The DTT solution was then replaced by 50 mM ammonium bicarbonate containing 100 mM Iodoacetamide for 45 minutes in the dark, with occasional vortexing. The gel pieces were then re-incubated in 50 mM ammonium bicarbonate/50% acetonitrile, and 100% acetonitrile under moderate shaking, followed by drying in speed-vac concentrator. The dry gel pieces were then rehydrated using 50 mM ammonium bicarbonate containing 10 ng/L trypsin and incubated overnight at 37° C. under low shaking. The resulting peptides were extracted twice with 5% formic acid/50 mM ammonium bicarbonate/50% acetonitrile and once with 100% acetonitrile under moderate shaking. Peptide mixture was then dried in a speed-vac, solubilized in 20 L of 0.1% formic acid/2% acetonitrile.

LC-MS/MS. The peptides mixture was analyzed by reversed phase liquid chromatography (LC) and MS (LC-MS/MS) using a NanoAcuity UPLC (Micromass/Waters, Milford, Mass.) coupled to a Q-TOF Ultima API MS (Micromass/Waters, Milford, Mass.), according to published procedures [157, 160-162]. Briefly, the peptides were loaded onto a 100 m×10 mm NanoAquity BEH130 C18 1.7 m UPLC column (Waters, Milford, Mass.) and eluted over a 150 minute gradient of 2-80% organic solvent (ACN containing 0.1% FA) at a flow rate of 400 nL/min. The aqueous solvent was 0.1% FA in HPLC water. The column was coupled to a Picotip Emitter Silicatip nano-electrospray needle (New Objective, Woburn, Mass.). MS data acquisition involved survey MS scans and automatic data dependent analysis (DDA) of the top three ions with the highest intensity ions with the charge of 2+, 3+ or 4+ ions. The MS/MS was triggered when the MS signal intensity exceeded 10 counts/second. In survey MS scans, the three most intense peaks were selected for collision-induced dissociation (CID) and fragmented until the total MS/MS ion counts reached 10,000 or for up to 6 seconds each. The entire procedure used was previously described [157, 160, 161]. Calibration was performed for both precursor and product ions using 1 pmol GluFib (Glu1-Fibrinopeptide B) standard peptide with the sequence EGVNDNEEGFFSAR (SEQ ID NO: 55) and the monoisotopic doubly-charged peak with m/z of 785.84.

Data processing and protein identification. The raw data were processed using ProteinLynx Global Server (PLGS, version 2.4) software as previously described [160]. The following parameters were used: background subtraction of polynomial order 5 adaptive with a threshold of 30%, two smoothings with a window of three channels in Savitzky-Golay mode and centroid calculation of top 80% of peaks based on a minimum peak width of 4 channels at half height. The resulting pkl files were submitted for database search and protein identification to the public Mascot database search (www.matrixscience.com, Matrix Science, London, UK) using the following parameters: databases from NCBI (all organisms, human proteins and rodent proteins for targeted identification of proteins), parent mass error of 1.3 Da, product ion error of 0.8 Da, enzyme used: trypsin, one missed cleavage, propionamide as cysteine fixed modification and Methionine oxidized as variable modification. To identify the false negative results, we used additional parameters such as different databases or organisms, a narrower error window for the parent mass error (1.2 and then 0.2 Da) and for the product ion error (0.6 Da), and up to two missed cleavage sites for trypsin. In addition, the pkl files were also searched against in-house PLGS database version 2.4 (www.waters.com) using searching parameters similar to the ones used for Mascot search. The Mascot and PLGS database search provided a list of proteins for each gel band. To eliminate false positive results, for the proteins identified by either one peptide or a mascot score lower than 25, we verified the MS/MS spectra that led to identification of a protein. The protein identified comprised of the amino acids:

(SEQ ID NO: 54)
EFDVILKAAGANKVAVIKAVRGATGLGLKEAKDLVESAPAALKEGVSKD
DAEALKKALEEAGAEVEVK

ACTIVEIMMUNE™ Activated DC are Superior to LPS Activated DC in Suppressing B16 Melanoma

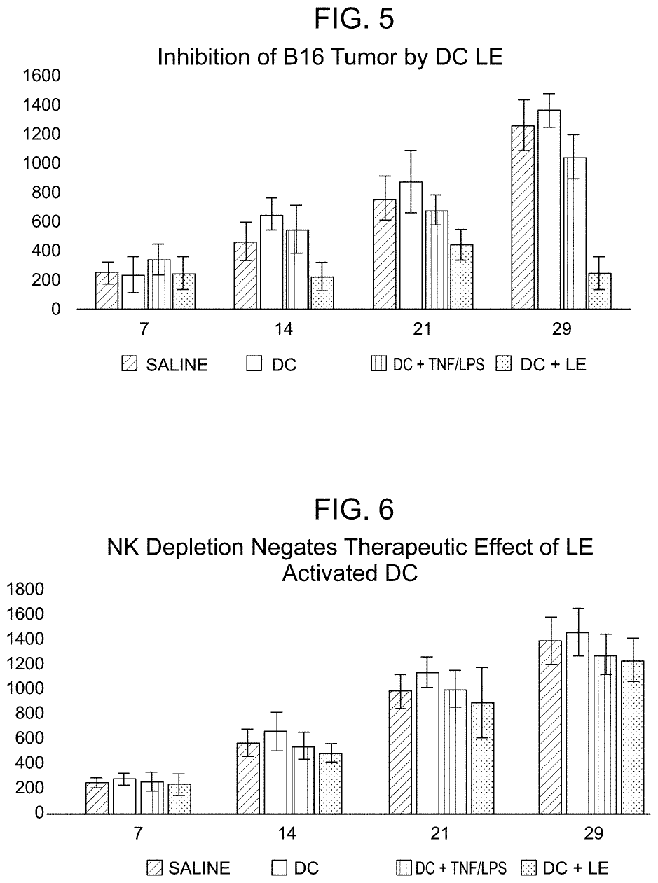

Mouse dendritic cells were generated by 6 day culture of bone marrow mononuclear cells in IL-4 and GM-CSF as previously described by us [163]. At day 6 DC were stimulated to mature by administration of 5 ug/ml LPS and 100 ng of TNF-alpha as a positive control. In the experimental group DC were treated with leukocyte extract (LE), which was ACTIVEIMMUNE™ (ImmunoActive™) obtained from Argo Farma, Mexico, was added to dendritic cells at a concentration of 10 micrograms per ml. C57/BL6 mice were inoculated with 500,000 B16 melanoma cells subcutaneously and tumors were allowed to grow for 7 days. Dendritic cells (positive and experimental controls) or saline were administered subcutaneously to animals at a concentration of 1 million cells per animal in a volume of 200 microliters. As seen in FIG. 5, a significant reduction in tumor size was observed in mice receiving DC that were pretreated with leukocyte extract. Tumor reduction efficacy was dependent on NK activity (8 mice per group). The experiment was repeated however NK cells were depleted by administration of injections every 3 days of anti-NK1.1 (PK136 mouse IgG2, hybridoma HB191; ATCC) (200 μg/dose) antibody intraperitoneally after administration of tumors. As seen in FIG. 6, antitumor efficacy was diminished upon the loss of NK cells.

### REFERENCES

1. Zhu N, Zhang D, Wang W, Li X, Yang B, Song J, Zhao X, Huang B, Shi W, Lu R et al: A Novel Coronavirus from Patients with Pneumonia in China, 2019. N Engl J Med 2020, 382(8):727-733.
2. Guo Y R, Cao Q D, Hong Z S, Tan Y Y, Chen S D, Jin H J, Tan K S, Wang D Y, Yan Y: The origin, transmission and clinical therapies on coronavirus disease 2019 (COVID-19) outbreak—an update on the status. Mil Med Res 2020, 7(1):11.
3. WHO WHO: Coronavirus disease (COVID-19) outbreak 2020:https://www.who.int/emergencies/diseases/novel-coronavirus-2019.
4. Zhang S, Diao M, Yu W, Pei L, Lin Z, Chen D: Estimation of the reproductive number of Novel Coronavirus (COVID-19) and the probable outbreak size on the Diamond Princess cruise ship: A data-driven analysis. Int J Infect Dis 2020.
5. Zhao S, Lin Q, Ran J, Musa S S, Yang G, Wang W, Lou Y, Gao D, Yang L, He D et al: Preliminary estimation of the basic reproduction number of novel coronavirus (2019-nCoV) in China, from 2019 to 2020: A data-driven analysis in the early phase of the outbreak. Int J Infect Dis 2020, 92:214-217.
6. Huang C, Wang Y, Li X, Ren L, Zhao J, Hu Y, Zhang L, Fan G, Xu J, Gu X et al: Clinical features of patients infected with 2019 novel coronavirus in Wuhan, China. Lancet 2020, 395(10223):497-506.
7. Wang D, Hu B, Hu C, Zhu F, Liu X, Zhang J, Wang B, Xiang H, Cheng Z, Xiong Y et al: Clinical Characteristics of 138 Hospitalized Patients With 2019 Novel Coronavirus-Infected Pneumonia in Wuhan, China. JAMA 2020.
8. Chen N, Zhou M, Dong X, Qu J, Gong F, Han Y, Qiu Y, Wang J, Liu Y, Wei Y et al: Epidemiological and clinical characteristics of 99 cases of 2019 novel coronavirus pneumonia in Wuhan, China: a descriptive study. Lancet 2020, 395(10223):507-513.
9. Grasselli G, Pesenti A, Cecconi M: Critical Care Utilization for the COVID-19 Outbreak in Lombardy, Italy: Early Experience and Forecast During an Emergency Response. JAMA 2020.
10. Wu C, Chen X, Cai Y, Xia J, Zhou X, Xu S, Huang H, Zhang L, Zhou X, Du C et al: Risk Factors Associated With Acute Respiratory Distress Syndrome and Death in Patients With Coronavirus Disease 2019 Pneumonia in Wuhan, China. JAMA Intern Med 2020.
11. Shi H, Han X, Jiang N, Cao Y, Alwalid O, Gu J, Fan Y, Zheng C: Radiological findings from 81 patients with COVID-19 pneumonia in Wuhan, China: a descriptive study. Lancet Infect Dis 2020.
12. Xu Z, Shi L, Wang Y, Zhang J, Huang L, Zhang C, Liu S, Zhao P, Liu H, Zhu L et al: Pathological findings of COVID-19 associated with acute respiratory distress syndrome. Lancet Respir Med 2020.
13. Tian S, Hu W, Niu L, Liu H, Xu H, Xiao S Y: Pulmonary pathology of early phase 2019 novel coronavirus (COVID-19) pneumonia in two patients with lung cancer. J Thorac Oncol 2020.
14. Martin-Fontecha A, Thomsen L L, Brett S, Gerard C, Lipp M, Lanzavecchia A, Sallusto F: Induced recruitment of NK cells to lymph nodes provides IFN-gamma for T(H)1 priming. Nat Immunol 2004, 5(12):1260-1265.
15. Morandi B, Bougras G, Muller W A, Ferlazzo G, Munz C: NK cells of human secondary lymphoid tissues enhance T cell polarization via IFN-gamma secretion. Eur J Immunol 2006, 36(9):2394-2400.
16. Ksienzyk A, Neumann B, Nandakumar R, Finsterbusch K, Grashoff M, Zawatzky R, Bernhardt G, Hauser H, Kroger A: IRF-1 expression is essential for natural killer cells to suppress metastasis. Cancer Res 2011, 71(20):6410-6418.
17. Lopez-Soto A, Gonzalez S, Smyth M J, Galluzzi L: Control of Metastasis by NK Cells. Cancer Cell 2017, 32(2):135-154.
18. Krasnova Y, Putz E M, Smyth M J, Souza-Fonseca-Guimaraes F: Bench to bedside: NK cells and control of metastasis. Clin Immunol 2017, 177:50-59.
19. Putz E M, Mayfosh A J, Kos K, Barkauskas D S, Nakamura K, Town L, Goodall K J, Yee D Y, Poon I K, Baschuk N et al: NK cell heparanase controls tumor invasion and immune surveillance. J Clin Invest 2017, 127(7):2777-2788.
20. Morvan M G, Lanier L L: NK cells and cancer: you can teach innate cells new tricks. Nat Rev Cancer 2016, 16(1):7-19.
21. Maccalli C, Giannarelli D, Chiarucci C, Cutaia O, Giacobini G, Hendrickx W, Amato G, Annesi D, Bedognetti D, Altomonte M et al: Soluble NKG2D ligands are biomarkers associated with the clinical outcome to immune checkpoint blockade therapy of metastatic melanoma patients. Oncoimmunology 2017, 6(7):e1323618.
22. Goding S R, Yu S, Bailey L M, Lotze M T, Basse P H: Adoptive transfer of natural killer cells promotes the anti-tumor efficacy of T cells. Clin Immunol 2017, 177:76-86.
23. Muraro E, Comaro E, Talamini R, Turchet E, Miolo G, Scalone S, Militello L, Lombardi D, Spazzapan S, Perin T et al: Improved Natural Killer cell activity and retained anti-tumor CD8(+) T cell responses contribute to the induction of a pathological complete response in HER2-positive breast cancer patients undergoing neoadjuvant chemotherapy. J Transl Med 2015, 13:204.
24. Lee S C, Srivastava R M, Lopez-Albaitero A, Ferrone S, Ferris R L: Natural killer (NK): dendritic cell (DC) cross talk induced by therapeutic monoclonal antibody triggers tumor antigen-specific T cell immunity. Immunol Res 2011, 50(2-3):248-254.
25. Beano A, Signorino E, Evangelista A, Brusa D, Mistrangelo M, Polimeni M A, Spadi R, Donadio M, Ciuffreda L, Matera L: Correlation between NK function and response to trastuzumab in metastatic breast cancer patients. J Transl Med 2008, 6:25.
26. Jaime-Ramirez A C, Mundy-Bosse B L, Kondadasula S, Jones N B, Roda J M, Mani A, Parihar R, Karpa V, Papenfuss T L, LaPerle K M et al: IL-12 enhances the antitumor actions of trastuzumab via NK cell IFN-gamma production. J Immunol 2011, 186(6):3401-3409.
27. Charlebois R, Allard B, Allard D, Buisseret L, Turcotte M, Pommey S, Chrobak P, Stagg J: PolyI:C and CpG Synergize with Anti-ErbB2 mAb for Treatment of Breast Tumors Resistant to Immune Checkpoint Inhibitors. Cancer Res 2017, 77(2):312-319.
28. Ugel S, Facciponte J G, De Sanctis F, Facciabene A: Targeting tumor vasculature: expanding the potential of DNA cancer vaccines. Cancer immunology, immunotherapy: CII 2015, 64(10):1339-1348.
29. Li L, Goedegebuure S P, Fleming T P, Gillanders W E: Developing a clinical development paradigm for translation of a mammaglobin-A DNA vaccine. Immunotherapy 2015:1-3.
30. Tiriveedhi V, Tucker N, Herndon J, Li L, Sturmoski M, Ellis M, Ma C, Naughton M, Lockhart A C, Gao F et al: Safety and preliminary evidence of biologic efficacy of a mammaglobin—a DNA vaccine in patients with stable metastatic breast cancer. Clinical cancer research: an official journal of the American Association for Cancer Research 2014, 20(23):5964-5975.
31. Heller R, Heller L C: Gene electrotransfer clinical trials. Advances in genetics 2015, 89:235-262.
32. Butterfield L H, Economou J S, Gamblin T C, Geller D A: Alpha fetoprotein DNA prime and adenovirus boost immunization of two hepatocellular cancer patients. Journal of translational medicine 2014, 12:86.
33. Sharpe M, Lynch D, Topham S, Major D, Wood J, Loudon P: Protection of mice from H5N1 influenza challenge by prophylactic DNA vaccination using particle mediated epidermal delivery. Vaccine 2007, 25(34):6392-6398.
34. Loudon P T, Yager E J, Lynch D T, Narendran A, Stagnar C, Franchini A M, Fuller J T, White P A, Nyuandi J, Wiley C A et al: GM-CSF increases mucosal and systemic immunogenicity of an H1N1 influenza DNA vaccine administered into the epidermis of non-human primates. PloS one 2010, 5(6):e11021.
35. Jones S, Evans K, McElwaine-Johnn H, Sharpe M, Oxford J, Lambkin-Williams R, Mant T, Nolan A, Zambon M, Ellis J et al: DNA vaccination protects against an influenza challenge in a double-blind randomised placebo-controlled phase 1b clinical trial. Vaccine 2009, 27(18):2506-2512.
36. Shah M A, Ali Z, Ahmad R, Qadri I, Fatima K, He N: DNA Mediated Vaccines Delivery Through Nanoparticles. Journal of nanoscience and nanotechnology 2015, 15(1):41-53.
37. Mpendo J, Mutua G, Nyombayire J, Ingabire R, Nanvubya A, Anzala O, Karita E, Hayes P, Kopycinski J, Dally L et al: A Phase I Double Blind, Placebo-Controlled, Randomized Study of the Safety and Immunogenicity of Electroporated HIV DNA with or without Interleukin 12 in Prime-Boost Combinations with an Ad35 HIV Vaccine in Healthy HIV-Seronegative African Adults. PloS one 2015, 10(8):e0134287.
38. Keane-Myers A M, Bell M, Hannaman D, Albrecht M: DNA electroporation of multi-agent vaccines conferring protection against select agent challenge: TriGrid delivery system. Methods in molecular biology 2014, 1121:325-336.
39. Hooper J W, Moon J E, Paolino K M, Newcomer R, McLain D E, Josleyn M, Hannaman D, Schmaljohn C: A Phase 1 clinical trial of Hantaan virus and Puumala virus M-segment DNA vaccines for haemorrhagic fever with renal syndrome delivered by intramuscular electroporation. Clinical microbiology and infection: the official publication of the European Society of Clinical Microbiology and Infectious Diseases 2014, 20 Suppl 5:110-117.
40. van Furth R, Cohn Z A: The origin and kinetics of mononuclear phagocytes. J Exp Med 1968, 128(3):415-435.
41. Wynn T A, Chawla A, Pollard J W: Macrophage biology in development, homeostasis and disease. Nature 2013, 496(7446):445-455.
42. Smith T D, Nagalla R R, Chen E Y, Liu W F: Harnessing macrophage plasticity for tissue regeneration. Adv Drug Deliv Rev 2017.
43. Vannella K M, Wynn T A: Mechanisms of Organ Injury and Repair by Macrophages. Annu Rev Physiol 2017, 79:593-617.
44. Boddupalli A, Zhu L, Bratlie K M: Methods for Implant Acceptance and Wound Healing: Material Selection and Implant Location Modulate Macrophage and Fibroblast Phenotypes. Adv Healthc Mater 2016, 5(20):2575-2594.
45. Snyder R J, Lantis J, Kirsner R S, Shah V, Molyneaux M, Carter M J: Macrophages: A review of their role in wound healing and their therapeutic use. Wound Repair Regen 2016, 24(4):613-629.
46. Gombozhapova A, Rogovskaya Y, Shurupov V, Rebenkova M, Kzhyshkowska J, Popov S V, Karpov R S, Ryabov V: Macrophage activation and polarization in post-infarction cardiac remodeling. J Biomed Sci 2017, 24(1): 13.
47. Hu Y, Zhang H, Lu Y, Bai H, Xu Y, Zhu X, Zhou R, Ben J, Xu Y, Chen Q: Class A scavenger receptor attenuates myocardial infarction-induced cardiomyocyte necrosis through suppressing M1 macrophage subset polarization. Basic Res Cardiol 2011, 106(6):1311-1328.
48. Ma Y, Halade G V, Zhang J, Ramirez T A, Levin D, Voorhees A, Jin Y F, Han H C, Manicone A M, Lindsey M L: Matrix metalloproteinase-28 deletion exacerbates cardiac dysfunction and rupture after myocardial infarction in mice by inhibiting M2 macrophage activation. Circ Res 2013, 112(4):675-688.
49. Lee C W, Hwang I, Park C S, Lee H, Park D W, Kang S J, Lee S W, Kim Y H, Park S W, Park S J: Macrophage heterogeneity of culprit coronary plaques in patients with acute myocardial infarction or stable angina. Am J Clin Pathol 2013, 139(3):317-322.
50. Yan X, Anzai A, Katsumata Y, Matsuhashi T, Ito K, Endo J, Yamamoto T, Takeshima A, Shinmura K, Shen W et al: Temporal dynamics of cardiac immune cell accumulation following acute myocardial infarction. J Mol Cell Cardiol 2013, 62:24-35.
51. Fernandez-Velasco M, Gonzalez-Ramos S, Bosca L: Involvement of monocytes/macrophages as key factors in the development and progression of cardiovascular diseases. Biochem J 2014, 458(2):187-193.
52. de Couto G, Liu W, Tseliou E, Sun B, Makkar N, Kanazawa H, Arditi M, Marban E: Macrophages mediate cardioprotective cellular postconditioning in acute myocardial infarction. J Clin Invest 2015, 125(8): 3147-3162.
53. Guiteras R, Flaquer M, Cruzado J M: Macrophage in chronic kidney disease. Clin Kidney J 2016, 9(6):765-771.
54. Meng X M, Tang P M, Li J, Lan H Y: Macrophage Phenotype in Kidney Injury and Repair. Kidney Dis (Basel) 2015, 1(2):138-146.
55. Yamamoto S, Zhong J, Yancey P G, Zuo Y, Linton M F, Fazio S, Yang H, Narita I, Kon V: Atherosclerosis following renal injury is ameliorated by pioglitazone and losartan via macrophage phenotype. Atherosclerosis 2015, 242(1):56-64.
56. Li C, Ding X Y, Xiang D M, Xu J, Huang X L, Hou F F, Zhou Q G: Enhanced M1 and Impaired M2 Macrophage Polarization and Reduced Mitochondrial Biogenesis via Inhibition of AMP Kinase in Chronic Kidney Disease. Cell Physiol Biochem 2015, 36(1):358-372.
57. Sun Y Y, Li X F, Meng X M, Huang C, Zhang L, Li J: Macrophage Phenotype in Liver Injury and Repair. Scand J Immunol 2017, 85(3):166-174.
58. Gratchev A, Kzhyshkowska J, Kothe K, Muller-Molinet I, Kannookadan S, Utikal J, Goerdt S: Mphi1 and Mphi2 can be re-polarized by Th2 or Th1 cytokines, respectively, and respond to exogenous danger signals. Immunobiology 2006, 211(6-8):473-486.
59. Mills C D: M1 and M2 Macrophages: Oracles of Health and Disease. Crit Rev Immunol 2012, 32(6):463-488.
60. Mills C D, Ley K: M1 and M2 macrophages: the chicken and the egg of immunity. J Innate Immun 2014, 6(6):716-726.
61. Alsaid H, Skedzielewski T, Rambo M V, Hunsinger K, Hoang B, Fieles W, Long E R, Tunstead J, Vugts D J, Cleveland M et al: Non invasive imaging assessment of the biodistribution of GSK2849330, an ADCC and CDC optimized anti HER3 mAb, and its role in tumor macrophage recruitment in human tumor-bearing mice. PLoS One 2017, 12(4):e0176075.
62. Josephs D H, Bax H J, Dodev T, Georgouli M, Nakamura M, Pellizzari G, Saul L, Karagiannis P, Cheung A, Herraiz C et al: Anti-Folate Receptor-alpha IgE but not IgG Recruits Macrophages to Attack Tumors via TNFalpha/MCP-1 Signaling. Cancer Res 2017, 77(5):1127-1141.
63. Velmurugan R, Challa D K, Ram S, Ober R I, Ward E S: Macrophage-Mediated Trogocytosis Leads to Death of Antibody-Opsonized Tumor Cells. Mol Cancer Ther 2016, 15(8):1879-1889.
64. Gul N, van Egmond M: Antibody-Dependent Phagocytosis of Tumor Cells by Macrophages: A Potent Effector Mechanism of Monoclonal Antibody Therapy of Cancer. Cancer Res 2015, 75(23):5008-5013.
65. Church A K, VanDerMeid K R, Baig N A, Baran A M, Witzig T E, Nowakowski G S, Zent C S: Anti-CD20 monoclonal antibody-dependent phagocytosis of chronic lymphocytic leukaemia cells by autologous macrophages. Clin Exp Immunol 2016, 183(1):90-101.
66. Shi Y, Fan X, Deng H, Brerski R J, Rycyzyn M, Jordan R E, Strohl W R, Zou Q, Zhang N, An Z: Trastuzumab triggers phagocytic killing of high HER2 cancer cells in vitro and in vivo by interaction with Fcgamma receptors on macrophages. J Immunol 2015, 194(9):4379-4386.
67. Weiskopf K, Weissman IL: Macrophages are critical effectors of antibody therapies for cancer. MAbs 2015, 7(2):303-310.
68. Oflazoglu E, Stone I J, Gordon K A, Grewal I S, van Rooijen N, Law C L, Gerber H P: Macrophages contribute to the antitumor activity of the anti-CD30 antibody SGN-30. Blood 2007, 110(13):4370-4372.
69. Osman R, Tacnet-Delorme P, Kleman J P, Millet A, Frachet P: Calreticulin Release at an Early Stage of Death Modulates the Clearance by Macrophages of Apoptotic Cells. Front Immunol 2017, 8:1034.
70. Feng M, Chen J Y, Weissman-Tsukamoto R, Volkmer J P, Ho P Y, McKenna K M, Cheshier S, Zhang M, Guo N, Gip P et al: Macrophages eat cancer cells using their own calreticulin as a guide: roles of TLR and Btk. Proc Natl Acad Sci USA 2015, 112(7):2145-2150.
71. Chao M P, Jaiswal S, Weissman-Tsukamoto R, Alizadeh A A, Gentles A J, Volkmer J, Weiskopf K, Willingham S B, Raveh T, Park C Y et al: Calreticulin is the dominant pro-phagocytic signal on multiple human cancers and is counterbalanced by CD47. Sci Transl Med 2010, 2(63):63ra94.
72. Murata Y, Kotani T, Ohnishi H, Matozaki T: The CD47-SIRPalpha signalling system: its physiological roles and therapeutic application. J Biochem 2014, 155(6):335-344.
73. Roberts D D, Kaur S, Soto-Pantoja D R: Therapeutic targeting of the thrombospondin-1 receptor CD47 to treat liver cancer. J Cell Commun Signal 2015, 9(1):101-102.
74. Liu J, Wang L, Zhao F, Tseng S, Narayanan C, Shura L, Willingham S, Howard M, Prohaska S, Volkmer J et al: Pre-Clinical Development of a Humanized Anti-CD47 Antibody with Anti-Cancer Therapeutic Potential. PLoS One 2015, 10(9):e0137345.
75. Weiskopf K, Jahchan N S, Schnorr P J, Cristea S, Ring A M, Maute R L, Volkmer A K, Volkmer J P, Liu J, Lim J S et al: CD47-blocking immunotherapies stimulate macrophage-mediated destruction of small-cell lung cancer. J Clin Invest 2016, 126(7):2610-2620.
76. Weiskopf K, Anderson K L, Ito D, Schnorr P J, Tomiyasu H, Ring A M, Bloink K, Efe J, Rue S, Lowery D et al: Eradication of Canine Diffuse Large B-Cell Lymphoma in a Murine Xenograft Model with CD47 Blockade and Anti-CD20. Cancer Immunol Res 2016, 4(12): 1072-1087.
77. Zeng D, Sun Q, Chen A, Fan J, Yang X, Xu L, Du P, Qiu W, Zhang W, Wang S et al: A fully human anti-CD47 blocking antibody with therapeutic potential for cancer. Oncotarget 2016, 7(50):83040-83050.
78. Liljefors M, Nilsson B, Mellstedt H, Frodin J E: Influence of varying doses of granulocyte-macrophage colony-stimulating factor on pharmacokinetics and antibody-dependent cellular cytotoxicity. Cancer Immunol Immunother 2008, 57(3):379-388.
79. Tarr P E: Granulocyte-macrophage colony-stimulating factor and the immune system. Med Oncol 1996, 13(3):133-140.
80. Ragnhammar P, Frodin J E, Trotta P P, Mellstedt H: Cytotoxicity of white blood cells activated by granulocyte-colony-stimulating factor, granulocyte/macrophage-colony-stimulating factor and macrophage-colony-stimulating factor against tumor cells in the presence of various monoclonal antibodies. Cancer Immunol Immunother 1994, 39(4):254-262.
81. Ragnhammar P: Anti-tumoral effect of GM-CSF with or without cytokines and monoclonal antibodies in solid tumors. Med Oncol 1996, 13(3):167-176.
82. Lin E Y, Nguyen A V, Russell R G, Pollard J W: Colony-stimulating factor 1 promotes progression of mammary tumors to malignancy. J Exp Med 2001, 193(6):727-740.
83. Aharinejad S, Paulus P, Sioud M, Hofmann M, Zins K, Schafer R, Stanley E R, Abraham D: Colony-stimulating factor-1 blockade by antisense oligonucleotides and small interfering RNAs suppresses growth of human mammary tumor xenografts in mice. Cancer Res 2004, 64(15):5378-5384.
84. Lin E Y, Li J F, Gnatovskiy L, Deng Y, Zhu L, Grzesik D A, Qian H, Xue X N, Pollard J W: Macrophages regulate the angiogenic switch in a mouse model of breast cancer. Cancer Res 2006, 66(23):11238-11246.
85. Lin E Y, Pollard J W: Tumor-associated macrophages press the angiogenic switch in breast cancer. Cancer Res 2007, 67(11):5064-5066.
86. Zhang W J, Chen C, Zhou Z H, Gao S T, Tee T J, Yang L Q, Xu Y Y, Pang T H, Xu X Y, Sun Q et al: Hypoxia-inducible factor-1 alpha Correlates with Tumor-Associated Macrophages Infiltration, Influences Survival of Gastric Cancer Patients. J Cancer 2017, 8(10):1818-1825.
87. Yuan X, Zhang J, Li D, Mao Y, Mo F, Du W, Ma X: Prognostic significance of tumor-associated macrophages in ovarian cancer: A meta-analysis. Gynecol Oncol 2017, 147(1):181-187.
88. Ma C, Horlad H, Ohnishi K, Nakagawa T, Yamada S, Kitada S, Motoshima T, Kamba T, Nakayama T, Fujimoto N et al: CD163-positive cancer cells are potentially associated with high malignant potential in clear cell renal cell carcinoma. Med Mol Morphol 2017.
89. Shi Y, Ping Y F, Zhou W, He Z C, Chen C, Bian B S, Zhang L, Chen L, Lan X, Zhang X C et al: Tumour-associated macrophages secrete pleiotrophin to promote PTPRZ1 signalling in glioblastoma stem cells for tumour growth. Nat Commun 2017, 8:15080.
90. Zhao X, Qu J, Sun Y, Wang J, Liu X, Wang F, Zhang H, Wang W, Ma X, Gao X et al: Prognostic significance of tumor-associated macrophages in breast cancer: a meta-analysis of the literature. Oncotarget 2017, 8(18):30576-30586.
91. Pander J, Heusinkveld M, van der Straaten T, Jordanova E S, Baak-Pablo R, Gelderblom H, Morreau H, van der Burg S H, Guchelaar H J, van Hall T: Activation of tumor-promoting type 2 macrophages by EGFR-targeting antibody cetuximab. Clin Cancer Res 2011, 17(17):5668-5673.
92. Clynes R A, Towers T L, Presta L G, Ravetch J V: Inhibitory Fc receptors modulate in vivo cytotoxicity against tumor targets. Nat Med 2000, 6(4):443-446.
93. Pricop L, Redecha P, Teillaud J L, Frey J, Fridman W H, Sautes-Fridman C, Salmon J E: Differential modulation of stimulatory and inhibitory Fc gamma receptors on human monocytes by Th1 and Th2 cytokines. J Immunol 2001, 166(1):531-537.
94. Tridandapani S, Siefker K, Teillaud J L, Carter J E, Wewers M D, Anderson C L: Regulated expression and inhibitory function of Fcgamma RIIb in human monocytic cells. J Biol Chem 2002, 277(7):5082-5089.
95. Joshi T, Ganesan L P, Cao X, Tridandapani S: Molecular analysis of expression and function of hFcgammaRIIb1 and b2 isoforms in myeloid cells. Mol Immunol 2006, 43(7):839-850.
96. Wijngaarden S, van de Winkel J G, Jacobs K M, Bijlsma J W, Lafeber F P, van Roon J A: A shift in the balance of inhibitory and activating Fcgamma receptors on monocytes toward the inhibitory Fcgamma receptor IIb is associated with prevention of monocyte activation in rheumatoid arthritis. Arthritis Rheum 2004, 50(12):3878-3887.
97. Butchar J P, Mehta P, Justiniano S E, Guenterberg K D, Kondadasula S V, Mo X, Chemudupati M, Kanneganti T D, Amer A, Muthusamy N et al: Reciprocal regulation of activating and inhibitory Fc{gamma} receptors by TLR7/8 activation: implications for tumor immunotherapy. Clin Cancer Res 2010, 16(7):2065-2075.
98. Fatehchand K, Ren L, Elavazhagan S, Fang H, Mo X, Vasilakos J P, Dietsch G N, Hershberg R M, Tridandapani S, Butchar J P: Toll-like Receptor 4 Ligands Down-regulate Fcgamma Receptor IIb (FcgammaRIIb) via MARCH3 Protein-mediated Ubiquitination. J Biol Chem 2016, 291(8):3895-3904.
99. Ghochikyan A, Pichugin A, Bagaev A, Davtyan A, Hovakimyan A, Tukhvatulin A, Davtyan H, Shcheblyakov D, Logunov D, Chulkina M et al: Targeting TLR-4 with a novel pharmaceutical grade plant derived agonist, Immunomax®, as a therapeutic strategy for metastatic breast cancer. J Transl Med 2014, 12:322.
100. Oronsky B, Paulmurugan R, Foygel K, Scicinski J, Knox S J, Peehl D, Zhao H, Ning S, Cabrales P, Summers T A, Jr. et al: RRx-001: a systemically non-toxic M2-to-M1 macrophage stimulating and prosensitizing agent in Phase II clinical trials. Expert Opin Investig Drugs 2017, 26(1):109-119.
101. Lee C, Bae S S, Joo H, Bae H: Melittin suppresses tumor progression by regulating tumor-associated macrophages in a Lewis lung carcinoma mouse model. Oncotarget 2017, 8(33):54951-54965.
102. Zhang Q, Yuan R F, Li X H, Xu T A, Zhang Y N, Yuan X L, Cui Y F, Shen W, Guan Q L, Sun X Y: Clinical Effects of CpG-Based Treatment on the Efficacy of Hepatocellular Carcinoma by Skewing Polarization Toward M1 Macrophage from M2. Cancer Biother Radiopharm 2017, 32(6):215-219.
103. Sato T, Shimosato T, Ueda A, Ishigatsubo Y, Klinman D M: Intrapulmonary Delivery of CpG Microparticles Eliminates Lung Tumors. Mol Cancer Ther 2015, 14(10):2198-2205.
104. Chiang C F, Chao T T, Su Y F, Hsu C C, Chien C Y, Chiu K C, Shiah S G, Lee C H, Liu S Y, Shieh Y S: Metformin-treated cancer cells modulate macrophage polarization through AMPK-NF-kappaB signaling. Oncotarget 2017, 8(13):20706-20718.
105. Kang H, Zhang J, Wang B, Liu M, Zhao J, Yang M, Li Y: Puerarin inhibits M2 polarization and metastasis of tumor-associated macrophages from NSCLC xenograft model via inactivating MEK/ERK 1/2 pathway. Int J Oncol 2017, 50(2):545-554.
106. Jia X, Yu F, Wang J, Iwanowycz S, Saaoud F, Wang Y, Hu J, Wang Q, Fan D: Emodin suppresses pulmonary metastasis of breast cancer accompanied with decreased macrophage recruitment and M2 polarization in the lungs. Breast Cancer Res Treat 2014, 148(2):291-302.
107. Xue N, Zhou Q, Ji M, Jin J, Lai F, Chen J, Zhang M, Jia J, Yang H, Zhang J et al: Chlorogenic acid inhibits glioblastoma growth through repolarizating macrophage from M2 to M1 phenotype. Sci Rep 2017, 7:39011.
108. Sloan E K, Priceman S J, Cox B F, Yu S, Pimentel M A, Tangkanangnukul V, Arevalo J M, Morizono K, Karanikolas B D, Wu L et al: The sympathetic nervous system induces a metastatic switch in primary breast cancer. Cancer Res 2010, 70(18):7042-7052.
109. Liu B, Wang X, Chen T Z, Li G L, Tan C C, Chen Y, Duan S Q: Polarization of M1 tumor associated macrophage promoted by the activation of TLR3 signal pathway. Asian Pac J Trop Med 2016, 9(5):484-488.
110. Liu Q, Tian Y, Zhao X, Jing H, Xie Q, Li P, Li D, Yan D, Zhu X: NMAAP1 Expressed in BCG-Activated Macrophage Promotes M1 Macrophage Polarization. Mol Cells 2015, 38(10):886-894.
111. Liu Y, Zhang L, Zhu X, Wang Y, Liu W, Gong W: Polysaccharide Agaricus blazei Murill stimulates myeloid derived suppressor cell differentiation from M2 to M1 type, which mediates inhibition of tumour immune-evasion via the Toll-like receptor 2 pathway. Immunology 2015, 146(3):379-391.
112. Yang Y, Zhang R, Xia F, Zou T, Huang A, Xiong S, Zhang J: LPS converts Gr-1(+)CD115(+) myeloid-derived suppressor cells from M2 to M1 via P38 MAPK. Exp Cell Res 2013, 319(12):1774-1783.
113. Sanchez-Quesada C, Lopez-Biedma A, Gaforio J J: Maslinic Acid enhances signals for the recruitment of macrophages and their differentiation to ml state. Evid Based Complement Alternat Med 2015, 2015:654721.
114. Dominguez-Soto A, de las Casas-Engel M, Bragado R, Medina-Echeverz J, Aragoneses-Fenoll L, Martin-Gayo E, van Rooijen N, Berraondo P, Toribio M L, Moro M A et al: Intravenous immunoglobulin promotes antitumor responses by modulating macrophage polarization. J Immunol 2014, 193(10):5181-5189.
115. Yin Y, Huang X, Lynn K D, Thorpe P E: Phosphatidylserine-targeting antibody induces M1 macrophage polarization and promotes myeloid-derived suppressor cell differentiation. Cancer Immunol Res 2013, 1(4):256-268.
116. Deng R, Wang S M, Yin T, Ye T H, Shen G B, Li L, Zhao J Y, Sang Y X, Duan X G, Wei Y Q: Dimethyl Sulfoxide Suppresses Mouse 4T1 Breast Cancer Growth by Modulating Tumor-Associated Macrophage Differentiation. J Breast Cancer 2014, 17(1):25-32.
117. Mitsuhashi A, Goto H, Kuramoto T, Tabata S, Yukishige S, Abe S, Hanibuchi M, Kakiuchi S, Saijo A, Aono Y et al: Surfactant protein A suppresses lung cancer progression by regulating the polarization of tumor-associated macrophages. Am J Pathol 2013, 182(5):1843-1853.
118. Coscia M, Quaglino E, Iezzi M, Curcio C, Pantaleoni F, Riganti C, Holen I, Monkkonen H, Boccadoro M, Forni G et al: Zoledronic acid repolarizes tumour-associated macrophages and inhibits mammary carcinogenesis by targeting the mevalonate pathway. J Cell Mol Med 2010, 14(12):2803-2815.
119. Eriksson F, Tsagozis P, Lundberg K, Parsa R, Mangsbo S M, Persson M A, Harris R A, Pisa P: Tumor-specific bacteriophages induce tumor destruction through activation of tumor-associated macrophages. J Immunol 2009, 182(5): 3105-3111.
120. Steinman R M, Cohn Z A: Identification of a novel cell type in peripheral lymphoid organs of mice. I. Morphology, quantitation, tissue distribution. The Journal of experimental medicine 1973, 137(5):1142-1162.
121. Banchereau J, Steinman R M: Dendritic cells and the control of immunity. Nature 1998, 392(6673):245-252.
122. Trombetta E S, Mellman I: Cell biology of antigen processing in vitro and in vivo. Annual review of immunology 2005, 23:975-1028.
123. Itano A A, Jenkins M K: Antigen presentation to naive CD4 T cells in the lymph node. Nature immunology 2003, 4(8):733-739.
124. Nestle F O, Alijagic S, Gilliet M, Sun Y, Grabbe S, Dummer R, Burg G, Schadendorf D: Vaccination of melanoma patients with peptide- or tumor lysate-pulsed dendritic cells. Nature medicine 1998, 4(3):328-332.
125. Chakraborty N G, Sporn J R, Tortora A F, Kurtzman S H, Yamase H, Ergin M T, Mukherji B: Immunization with a tumor-cell-lysate-loaded autologous-antigen-presenting-cell-based vaccine in melanoma. Cancer immunology, immunotherapy: CII 1998, 47(1):58-64.
126. Wang F, Bade E, Kuniyoshi C, Spears L, Jeffery G, Marty V, Groshen S, Weber J: Phase I trial of a MART-1 peptide vaccine with incomplete Freund's adjuvant for resected high-risk melanoma. Clinical cancer research: an official journal of the American Association for Cancer Research 1999, 5(10):2756-2765.
127. Thurner B, Haendle I, Roder C, Dieckmann D, Keikavoussi P, Jonuleit H, Bender A, Maczek C, Schreiner D, von den Driesch P et al: Vaccination with mage-3A1 peptide-pulsed mature, monocyte-derived dendritic cells expands specific cytotoxic T cells and induces regression of some metastases in advanced stage IV melanoma. The Journal of experimental medicine 1999, 190(11):1669-1678.
128. Thomas R, Chambers M, Boytar R, Barker K, Cavanagh L L, MacFadyen S, Smithers M, Jenkins M, Andersen J: Immature human monocyte-derived dendritic cells migrate rapidly to draining lymph nodes after intradermal injection for melanoma immunotherapy. Melanoma research 1999, 9(5):474-481.
129. Mackensen A, Herbst B, Chen J L, Kohler G, Noppen C, Herr W, Spagnoli G C, Cerundolo V, Lindemann A: Phase I study in melanoma patients of a vaccine with peptide-pulsed dendritic cells generated in vitro from CD34(+) hematopoietic progenitor cells. International journal of cancer Journal international du cancer 2000, 86(3):385-392.
130. Panelli M C, Wunderlich J, Jeffries J, Wang E, Mixon A, Rosenberg S A, Marincola FM: Phase 1 study in patients with metastatic melanoma of immunization with dendritic cells presenting epitopes derived from the melanoma-associated antigens MART-1 and gp100. Journal of immunotherapy 2000, 23(4):487-498.
131. Schuler-Thurner B, Dieckmann D, Keikavoussi P, Bender A, Maczek C, Jonuleit H, Roder C, Haendle I, Leisgang W, Dunbar R et al: Mage-3 and influenza-matrix peptide-specific cytotoxic T cells are inducible in terminal stage HLA-A2.1+ melanoma patients by mature monocyte-derived dendritic cells. Journal of immunology 2000, 165(6):3492-3496.
132. Lau R, Wang F, Jeffery G, Marty V, Kuniyoshi J, Bade E, Ryback M E, Weber J: Phase I trial of intravenous peptide-pulsed dendritic cells in patients with metastatic melanoma. Journal of immunotherapy 2001, 24(1):66-78.
133. Banchereau J, Palucka A K, Dhodapkar M, Burkeholder S, Taquet N, Rolland A, Taquet S, Coquery S, Wittkowski K M, Bhardwaj N et al: Immune and clinical responses in patients with metastatic melanoma to CD34(+) progenitor-derived dendritic cell vaccine. Cancer research 2001, 61(17):6451-6458.
134. Schuler-Thurner B, Schultz E S, Berger T G, Weinlich G, Ebner S, Woerl P, Bender A, Feuerstein B, Fritsch P O, Romani N et al: Rapid induction of tumor-specific type 1 T helper cells in metastatic melanoma patients by vaccination with mature, cryopreserved, peptide-loaded monocyte-derived dendritic cells. The Journal of experimental medicine 2002, 195(10):1279-1288.
135. Palucka A K, Dhodapkar M V, Paczesny S, Burkeholder S, Wittkowski K M, Steinman R M, Fay J, Banchereau J: Single injection of CD34+ progenitor-derived dendritic cell vaccine can lead to induction of T-cell immunity in patients with stage IV melanoma. Journal of immunotherapy 2003, 26(5):432-439.
136. Bedrosian I, Mick R, Xu S, Nisenbaum H, Faries M, Zhang P, Cohen P A, Koski G, Czerniecki B J: Intranodal administration of peptide-pulsed mature dendritic cell vaccines results in superior CD8+ T-cell function in melanoma patients. Journal of clinical oncology: official journal of the American Society of Clinical Oncology 2003, 21(20):3826-3835.
137. Slingluff C L, Jr., Petroni G R, Yamshchikov G V, Barnd D L, Eastham S, Galavotti H, Patterson J W, Deacon D H, Hibbitts S, Teates D et al: Clinical and immunologic results of a randomized phase II trial of vaccination using four melanoma peptides either administered in granulocyte-macrophage colony-stimulating factor in adjuvant or pulsed on dendritic cells. Journal of clinical oncology: official journal of the American Society of Clinical Oncology 2003, 21(21):4016-4026.
138. Hersey P, Menzies S W, Halliday G M, Nguyen T, Farrelly M L, DeSilva C, Lett M: Phase I/II study of treatment with dendritic cell vaccines in patients with disseminated melanoma. Cancer immunology, immunotherapy: CII 2004, 53(2):125-134.
139. Vilella R, Benitez D, Mila J, Lozano M, Vilana R, Pomes J, Tomas X, Costa J, Vilalta A, Malvehy J et al: Pilot study of treatment of biochemotherapy-refractory stage IV melanoma patients with autologous dendritic cells pulsed with a heterologous melanoma cell line lysate. Cancer immunology, immunotherapy: CII 2004, 53(7):651-658.
140. Palucka A K, Connolly J, Ueno H, Kohl J, Paczesny S, Dhodapkar M, Fay J, Banchereau J: Spontaneous proliferation and type 2 cytokine secretion by CD4+ T cells in patients with metastatic melanoma vaccinated with antigen-pulsed dendritic cells. Journal of clinical immunology 2005, 25(3):288-295.
141. Banchereau J, Ueno H, Dhodapkar M, Connolly J, Finholt J P, Klechevsky E, Blanck J P, Johnston D A, Palucka A K, Fay J: Immune and clinical outcomes in patients with stage IV melanoma vaccinated with peptide-pulsed dendritic cells derived from CD34+ progenitors and activated with type I interferon. Journal of immunotherapy 2005, 28(5):505-516.
142. Trakatelli M, Toungouz M, Blocklet D, Dodoo Y, Gordower L, Laporte M, Vereecken P, Sales F, Mortier L, Mazouz N et al: A new dendritic cell vaccine generated with interleukin-3 and interferon-beta induces CD8+ T cell responses against NA17-A2 tumor peptide in melanoma patients. Cancer immunology, immunotherapy: CII 2006, 55(4):469-474.
143. Salcedo M, Bercovici N, Taylor R, Vereecken P, Massicard S, Duriau D, Vernel-Pauillac F, Boyer A, Baron-Bodo V, Mallard E et al: Vaccination of melanoma patients using dendritic cells loaded with an allogeneic tumor cell lysate. Cancer immunology, immunotherapy: CII 2006, 55(7):819-829.
144. Linette G P, Zhang D, Hodi F S, Jonasch E P, Longerich S, Stowell C P, Webb I J, Daley H, Soiffer R J, Cheung A M et al: Immunization using autologous dendritic cells pulsed with the melanoma-associated antigen gp100-derived G280-9V peptide elicits CD8+ immunity. Clinical cancer research: an official journal of the American Association for Cancer Research 2005, 11(21):7692-7699.
145. Escobar A, Lopez M, Serrano A, Ramirez M, Perez C, Aguirre A, Gonzalez R, Alfaro J, Larrondo M, Fodor M et al: Dendritic cell immunizations alone or combined with low doses of interleukin-2 induce specific immune responses in melanoma patients. Clinical and experimental immunology 2005, 142(3):555-568.
146. Tuettenberg A, Becker C, Huter E, Knop J, Enk A H, Jonuleit H: Induction of strong and persistent MelanA/MART-1-specific immune responses by adjuvant dendritic cell-based vaccination of stage I I melanoma patients. International journal of cancer Journal international du cancer 2006, 118(10):2617-2627.
147. Schadendorf D, Ugurel S, Schuler-Thurner B, Nestle F O, Enk A, Brocker E B, Grabbe S, Rittgen W, Edler L, Sucker A et al: Dacarbazine (DTIC) versus vaccination with autologous peptide-pulsed dendritic cells (D C) in first-line treatment of patients with metastatic melanoma: a randomized phase III trial of the D C study group of the DeCOG. Annals of oncology: official journal of the European Society for Medical Oncology/ESMO 2006, 17(4):563-570.
148. Di Pucchio T, Pilla L, Capone I, Ferrantini M, Montefiore E, Urbani F, Patuzzo R, Pennacchioli E, Santinami M, Cova A et al: Immunization of stage I V melanoma patients with Melan-A/MART-1 and gp100 peptides plus IFN-alpha results in the activation of specific CD8(+) T cells and monocyte/dendritic cell precursors. Cancer research 2006, 66(9):4943-4951.
149. Nakai N, Asai J, Ueda E, Takenaka H, Katoh N, Kishimoto S: Vaccination of Japanese patients with advanced melanoma with peptide, tumor lysate or both peptide and tumor lysate-pulsed mature, monocyte-derived dendritic cells. The Journal of dermatology 2006, 33(7):462-472.
150. Palucka A K, Ueno H, Connolly J, Kerneis-Norvell F, Blanck J P, Johnston D A, Fay J, Banchereau J: Dendritic cells loaded with killed allogeneic melanoma cells can induce objective clinical responses and MART-1 specific CD8+ T-cell immunity. Journal of immunotherapy 2006, 29(5):545-557.
151. Lesimple T, Neidhard E M, Vignard V, Lefeuvre C, Adamski H, Labarriere N, Carsin A, Monnier D, Collet B, Clapisson G et al: Immunologic and clinical effects of injecting mature peptide-loaded dendritic cells by intralymphatic and intranodal routes in metastatic melanoma patients. Clinical cancer research: an official journal of the American Association for Cancer Research 2006, 12(24):7380-7388.
152. Guo J, Zhu J, Sheng X, Wang X, Qu L, Han Y, Liu Y, Zhang H, Huo L, Zhang S et al: Intratumoral injection of dendritic cells in combination with local hyperthermia induces systemic antitumor effect in patients with advanced melanoma. International journal of cancer Journal international du cancer 2007, 120(11):2418-2425.
153. O'Rourke M G, Johnson M K, Lanagan C M, See J L, O'Connor L E, Slater G J, Thomas D, Lopez J A, Martinez N R, Ellem K A et al: Dendritic cell immunotherapy for stage I V melanoma. Melanoma research 2007, 17(5):316-322.
154. Bercovici N, Haicheur N, Massicard S, Vernel-Pauillac F, Adotevi O, Landais D, Gorin I, Robert C, Prince H M, Grob J J et al: Analysis and characterization of antitumor T-cell response after administration of dendritic cells loaded with allogeneic tumor lysate to metastatic melanoma patients. Journal of immunotherapy 2008, 31(1):101-112.
155. Hersey P, Halliday G M, Farrelly M L, DeSilva C, Lett M, Menzies S W: Phase I/I I study of treatment with matured dendritic cells with or without low dose IL-2 in patients with disseminated melanoma. Cancer immunology, immunotherapy: CII 2008, 57(7): 1039-1051.
156. von Euw E M, Barrio M M, Furman D, Levy E M, Bianchini M, Peguillet I, Lantz O, Vellice A, Kohan A, Chacon M et al: A phase I clinical study of vaccination of melanoma patients with dendritic cells loaded with allogeneic apoptotic/necrotic melanoma cells. Analysis of toxicity and immune response to the vaccine and of IL-10-1082 promoter genotype as predictor of disease progression. Journal of translational medicine 2008, 6:6.
157. Carrasco J, Van Pel A, Neyns B, Lethe B, Brasseur F, Renkvist N, van der Bruggen P, van Baren N, Paulus R, Thielemans K et al: Vaccination of a melanoma patient with mature dendritic cells pulsed with MAGE-3 peptides triggers the activity of nonvaccine anti-tumor cells. Journal of immunology 2008, 180(5):3585-3593.
158. Redman B G, Chang A E, Whitfield J, Esper P, Jiang G, Braun T, Roessler B, Mule J J: Phase Ib trial assessing autologous, tumor-pulsed dendritic cells as a vaccine administered with or without IL-2 in patients with metastatic melanoma. Journal of immunotherapy 2008, 31(6):591-598.
159. Daud A I, Mirza N, Lenox B, Andrews S, Urbas P, Gao G X, Lee J H, Sondak V K, Riker A I, Deconti R C et al: Phenotypic and functional analysis of dendritic cells and clinical outcome in patients with high-risk melanoma treated with adjuvant granulocyte macrophage colony-stimulating factor. Journal of clinical oncology: official journal of the American Society of Clinical Oncology 2008, 26(19):3235-3241.
160. Engell-Noerregaard L, Hansen T H, Andersen M H, Thor Straten P, Svane I M: Review of clinical studies on dendritic cell-based vaccination of patients with malignant melanoma: assessment of correlation between clinical response and vaccine parameters. Cancer immunology, immunotherapy: CII 2009, 58(1):1-14.
161. Nakai N, Katoh N, Germeraad W T, Kishida T, Ueda E, Takenaka H, Mazda O, Kishimoto S: Immunohistological analysis of peptide-induced delayed-type hypersensitivity in advanced melanoma patients treated with melanoma antigen-pulsed mature monocyte-derived dendritic cell vaccination. Journal of dermatological science 2009, 53(1):40-47.
162. Dillman R O, Selvan S R, Schiltz P M, McClay E F, Barth N M, DePriest C, de Leon C, Mayorga C, Cornforth A N, Allen K: Phase I I trial of dendritic cells loaded with antigens from self-renewing, proliferating autologous tumor cells as patient-specific antitumor vaccines in patients with metastatic melanoma: final report. Cancer biotherapy & radiopharmaceuticals 2009, 24(3):311-319.
163. Chang J W, Hsieh J J, Shen Y C, Ho E, Chuang C K, Chen Y R, Liao S K, Chen J S, Leong S P, Hou M M et al: Immunotherapy with dendritic cells pulsed by autologous dactinomycin-induced melanoma apoptotic bodies for patients with malignant melanoma. Melanoma research 2009, 19(5):309-315.
164. Trepiakas R, Berntsen A, Hadrup S R, Bjorn J, Geertsen P F, Straten P T, Andersen M H, Pedersen A E, Soleimani A, Lorentzen T et al: Vaccination with autologous dendritic cells pulsed with multiple tumor antigens for treatment of patients with malignant melanoma: results from a phase I/I I trial. Cytotherapy 2010, 12(6):721-734.
165. Jacobs J F, Punt C J, Lesterhuis W J, Sutmuller R P, Brouwer H M, Scharenborg N M, Klasen I S, Hilbrands L B, Figdor C G, de Vries I J et al: Dendritic cell vaccination in combination with anti-CD25 monoclonal antibody treatment: a phase I/I I study in metastatic melanoma patients. Clinical cancer research: an official journal of the American Association for Cancer Research 2010, 16(20):5067-5078.
166. Ribas A, Camacho L H, Lee S M, Hersh E M, Brown C K, Richards J M, Rodriguez M J, Prieto V G, Glaspy J A, Oseguera D K et al: Multicenter phase I I study of matured dendritic cells pulsed with melanoma cell line lysates in patients with advanced melanoma. Journal of translational medicine 2010, 8:89.
167. Ridolfi L, Petrini M, Fiammenghi L, Granato A M, Ancarani V, Pancisi E, Scarpi E, Guidoboni M, Migliori G, Sanna S et al: Unexpected high response rate to traditional therapy after dendritic cell-based vaccine in advanced melanoma: update of clinical outcome and subgroup analysis. Clinical &developmental immunology 2010, 2010:504979.
168. Cornforth A N, Fowler A W, Carbonell D J, Dillman R O: Resistance to the proapoptotic effects of interferon-gamma on melanoma cells used in patient-specific dendritic cell immunotherapy is associated with improved overall survival. Cancer immunology, immunotherapy: CII 2011, 60(1): 123-131.
169. Lesterhuis W J, Schreibelt G, Scharenborg N M, Brouwer H M, Gerritsen M J, Croockewit S, Coulie P G, Torensma R, Adema G J, Figdor C G et al: Wild-type and modified gp100 peptide-pulsed dendritic cell vaccination of advanced melanoma patients can lead to long-term clinical responses independent of the peptide used. Cancer immunology, immunotherapy: CII 2011, 60(2):249-260.
170. Bjoern J, Brimnes M K, Andersen M H, Thor Straten P, Svane I M: Changes in peripheral blood level of regulatory T cells in patients with malignant melanoma during treatment with dendritic cell vaccination and low-dose IL-2. Scandinavian journal of immunology 2011, 73(3):222-233.
171. Steele J C, Rao A, Marsden J R, Armstrong C J, Berhane S, Billingham U, Graham N, Roberts C, Ryan G, Uppal H et al: Phase VII trial of a dendritic cell vaccine transfected with DNA encoding melan A and gp100 for patients with metastatic melanoma. Gene therapy 2011, 18(6):584-593.
172. Kim D S, Kim D H, Goo B, Cho Y H, Park J M, Lee T H, Kim H O, Kim H S, Lee H, Lee J D et al: Immunotherapy of malignant melanoma with tumor lysate-pulsed autologous monocyte-derived dendritic cells. Yonsei medical journal 2011, 52(6):990-998.
173. Ellebaek E, Engell-Noerregaard L, Iversen T Z, Froesig™, Munir S, Hadrup S R, Andersen M H, Svane I M: Metastatic melanoma patients treated with dendritic cell vaccination, Interleukin-2 and metronomic cyclophosphamide: results from a phase I I trial. Cancer immunology, immunotherapy: CII 2012, 61(10): 1791-1804.
174. Dillman R O, Cornforth A N, Depriest C, McClay E F, Amatruda T T, de Leon C, Ellis R E, Mayorga C, Carbonell D, Cubellis J M: Tumor stem cell antigens as consolidative active specific immunotherapy: a randomized phase I I trial of dendritic cells versus tumor cells in patients with metastatic melanoma. Journal of immunotherapy 2012, 35(8):641-649.
175. Dannull J, Haley N R, Archer G, Nair S, Boczkowski D, Harper M, De Rosa N, Pickett N, Mosca P J, Burchette J et al: Melanoma immunotherapy using mature DCs expressing the constitutive proteasome. The Journal of clinical investigation 2013, 123(7):3135-3145.
176. Finkelstein S E, Iclozan C, Bui M M, Cotter M J, Ramakrishnan R, Ahmed J, Noyes D R, Cheong D, Gonzalez R J, Heysek R V et al: Combination of external beam radiotherapy (EBRT) with intratumoral injection of dendritic cells as neo-adjuvant treatment of high-risk soft tissue sarcoma patients. International journal of radiation oncology, biology, physics 2012, 82(2):924-932.
177. Stift A, Sachet M, Yagubian R, Bittermann C, Dubsky P, Brostjan C, Pfragner R, Niederle B, Jakesz R, Gnant M et al: Dendritic cell vaccination in medullary thyroid carcinoma. Clinical cancer research: an official journal of the American Association for Cancer Research 2004, 10(9):2944-2953.
178. Kuwabara K, Nishishita T, Morishita M, Oyaizu N, Yamashita S, Kanematsu T, Obara T, Mimura Y, Inoue Y, Kaminishi M et al: Results of a phase I clinical study using dendritic cell vaccinations for thyroid cancer. Thyroid: official journal of the American Thyroid Association 2007, 17(1):53-58.
179. Bachleitner-Hofmann T, Friedl J, Hassler M, Hayden H, Dubsky P, Sachet M, Rieder E, Pfragner R, Brostjan C, Riss S et al: Pilot trial of autologous dendritic cells loaded with tumor lysate(s) from allogeneic tumor cell lines in patients with metastatic medullary thyroid carcinoma. Oncology reports 2009, 21(6):1585-1592.
180. Yu J S, Wheeler C J, Zeltzer P M, Ying H, Finger D N, Lee P K, Yong W H, Incardona F, Thompson R C, Riedinger M S et al: Vaccination of malignant glioma patients with peptide-pulsed dendritic cells elicits systemic cytotoxicity and intracranial T-cell infiltration. Cancer research 2001, 61(3):842-847.
181. Yamanaka R, Abe T, Yajima N, Tsuchiya N, Homma J, Kobayashi T, Narita M, Takahashi M, Tanaka R: Vaccination of recurrent glioma patients with tumour lysate-pulsed dendritic cells elicits immune responses: results of a clinical phase I/I I trial. British journal of cancer 2003, 89(7):1172-1179.
182. Yu J S, Liu G, Ying H, Yong W H, Black K L, Wheeler C J: Vaccination with tumor lysate-pulsed dendritic cells elicits antigen-specific, cytotoxic T-cells in patients with malignant glioma. Cancer research 2004, 64(14):4973-4979.
183. Yamanaka R, Honma J, Tsuchiya N, Yajima N, Kobayashi T, Tanaka R: Tumor lysate and IL-18 loaded dendritic cells elicits Th1 response, tumor-specific CD8+ cytotoxic T cells in patients with malignant glioma. Journal of neuro-oncology 2005, 72(2):107-113.
184. Yamanaka R, Homma J, Yajima N, Tsuchiya N, Sano M, Kobayashi T, Yoshida S, Abe T, Narita M, Takahashi M et al: Clinical evaluation of dendritic cell vaccination for patients with recurrent glioma: results of a clinical phase VII trial. Clinical cancer research: an official journal of the American Association for Cancer Research 2005, 11(11):4160-4167.
185. Liau L M, Prins R M, Kiertscher S M, Odesa S K, Kremen T J, Giovannone A J, Lin J W, Chute D J, Mischel P S, Cloughesy T F et al: Dendritic cell vaccination in glioblastoma patients induces systemic and intracranial T-cell responses modulated by the local central nervous system tumor microenvironment. Clinical cancer research: an official journal of the American Association for Cancer Research 2005, 11(15):5515-5525.
186. Walker D G, Laherty R, Tomlinson F H, Chuah T, Schmidt C: Results of a phase I dendritic cell vaccine trial for malignant astrocytoma: potential interaction with adjuvant chemotherapy. Journal of clinical neuroscience: official journal of the Neurosurgical Society of Australasia 2008, 15(2):114-121.
187. Leplina O Y, Stupak V V, Kozlov Y P, Pendyurin I V, Nikonov S D, Tikhonova M A, Sycheva N V, Ostanin A A, Chernykh E R: Use of interferon-alpha-induced dendritic cells in the therapy of patients with malignant brain gliomas. Bulletin of experimental biology and medicine 2007, 143(4):528-534.
188. De Vleeschouwer S, Fieuws S, Rutkowski S, Van Calenbergh F, Van Loon J, Goffin J, Sciot R, Wilms G, Demaerel P, Warmuth-Metz M et al: Postoperative adjuvant dendritic cell-based immunotherapy in patients with relapsed glioblastoma multiforme. Clinical cancer research: an official journal of the American Association for Cancer Research 2008, 14(10):3098-3104.
189. Ardon H, De Vleeschouwer S, Van Calenbergh F, Claes L, Kramm C M, Rutkowski S, Wolff J E, Van Gool S W: Adjuvant dendritic cell-based tumour vaccination for children with malignant brain tumours. Pediatric blood & cancer 2010, 54(4):519-525.
190. Prins R M, Soto H, Konkankit V, Odesa S K, Eskin A, Yong W H, Nelson S F, Liau L M: Gene expression profile correlates with T-cell infiltration and relative survival in glioblastoma patients vaccinated with dendritic cell immunotherapy. Clinical cancer research: an official journal of the American Association for Cancer Research 2011, 17(6):1603-1615.
191. Okada H, Kalinski P, Ueda R, Hoji A, Kohanbash G, Donegan T E, Mintz A H, Engh J A, Bartlett D L, Brown C K et al: Induction of CD8+ T-cell responses against novel glioma-associated antigen peptides and clinical activity by vaccinations with {alpha}-type 1 polarized dendritic cells and polyinosinic-polycytidylic acid stabilized by lysine and carboxymethylcellulose in patients with recurrent malignant glioma. Journal of clinical oncology: official journal of the American Society of Clinical Oncology 2011, 29(3):330-336.
192. Fadul C E, Fisher J L, Hampton T H, Lallana E C, Li Z, Gui J, Szczepiorkowski Z M, Tosteson T D, Rhodes C H, Wishart H A et al: Immune response in patients with newly diagnosed glioblastoma multiforme treated with intranodal autologous tumor lysate-dendritic cell vaccination after radiation chemotherapy. Journal of immunotherapy 2011, 34(4): 382-389.
193. Chang C N, Huang Y C, Yang D M, Kikuta K, Wei K J, Kubota T, Yang W K: A phase VII clinical trial investigating the adverse and therapeutic effects of a postoperative autologous dendritic cell tumor vaccine in patients with malignant glioma. Journal of clinical neuroscience: official journal of the Neurosurgical Society of Australasia 2011, 18(8):1048-1054.
194. Cho D Y, Yang W K, Lee H C, Hsu D M, Lin H L, Lin S Z, Chen C C, Ham H J, Liu C L, Lee W Y et al: Adjuvant immunotherapy with whole-cell lysate dendritic cells vaccine for glioblastoma multiforme: a phase I I clinical trial. World neurosurgery 2012, 77(5-6):736-744.
195. Iwami K, Shimato S, Ohno M, Okada H, Nakahara N, Sato Y, Yoshida J, Suzuki S, Nishikawa H, Shiku H et al: Peptide-pulsed dendritic cell vaccination targeting interleukin-13 receptor alpha2 chain in recurrent malignant glioma patients with HLA-A*24/A*02 allele. Cytotherapy 2012, 14(6):733-742.
196. Fong B, Jin R, Wang X, Safaee M, Lisiero D N, Yang I, Li G, Liau L M, Prins R M: Monitoring of regulatory T cell frequencies and expression of CTLA-4 on T cells, before and after D C vaccination, can predict survival in GBM patients. PloS one 2012, 7(4):e32614.
197. De Vleeschouwer S, Ardon H, Van Calenbergh F, Sciot R, Wilms G, van Loon J, Goffin J, Van Gool S: Stratification according to HGG-IMMUNO RPA model predicts outcome in a large group of patients with relapsed malignant glioma treated by adjuvant postoperative dendritic cell vaccination. Cancer immunology, immunotherapy: C112012, 61(11):2105-2112.
198. Phuphanich S, Wheeler C J, Rudnick J D, Mazer M, Wang H, Nuno M A, Richardson J E, Fan X, Ji J, Chu R M et al: Phase I trial of a multi-epitope-pulsed dendritic cell vaccine for patients with newly diagnosed glioblastoma. Cancer immunology, immunotherapy: CII 2013, 62(1):125-135.
199. Akiyama Y, Oshita C, Kume A, lizuka A, Miyata H, Komiyama M, Ashizawa T, Yagoto M, Abe Y, Mitsuya K et al: alpha-type-1 polarized dendritic cell-based vaccination in recurrent high-grade glioma: a phase I clinical trial. BMC cancer 2012, 12:623.
200. Prins R M, Wang X, Soto H, Young E, Lisiero D N, Fong B, Everson R, Yong W H, Lai A, Li G et al: Comparison of glioma-associated antigen peptide-loaded versus autologous tumor lysate-loaded dendritic cell vaccination in malignant glioma patients. Journal of immunotherapy 2013, 36(2):152-157.
201. Shah A H, Bregy A, Heros D O, Komotar R J, Goldberg J: Dendritic cell vaccine for recurrent high-grade gliomas in pediatric and adult subjects: clinical trial protocol. Neurosurgery 2013, 73(5):863-867.
202. Reichardt V L, Okada C Y, Liso A, Benike C J, Stockerl-Goldstein K E, Engleman E G, Blume K G, Levy R: Idiotype vaccination using dendritic cells after autologous peripheral blood stem cell transplantation for multiple myeloma—a feasibility study. Blood 1999, 93(7):2411-2419.
203. Lim S H, Bailey-Wood R: Idiotypic protein-pulsed dendritic cell vaccination in multiple myeloma. International journal of cancer Journal international du cancer 1999, 83(2):215-222.
204. Motta M R, Castellani S, Rizzi S, Curti A, Gubinelli F, Fogli M, Ferri E, Cellini C, Baccarani M, Lemoli R M: Generation of dendritic cells from CD14+ monocytes positively selected by immunomagnetic adsorption for multiple myeloma patients enrolled in a clinical trial of anti-idiotype vaccination. British journal of haematology 2003, 121(2):240-250.
205. Reichardt V L, Milazzo C, Brugger W, Einsele H, Kanz L, Brossart P: Idiotype vaccination of multiple myeloma patients using monocyte-derived dendritic cells. Haematologica 2003, 88(10):1139-1149.
206. Guardino A E, Rajapaksa R, Ong K H, Sheehan K, Levy R: Production of myeloid dendritic cells (D C) pulsed with tumor-specific idiotype protein for vaccination of patients with multiple myeloma. Cytotherapy 2006, 8(3):277-289.
207. Lacy M Q, Mandrekar S, Dispenzieri A, Hayman S, Kumar S, Buadi F, Dingli D, Litzow M, Wettstein P, Padley D et al: Idiotype-pulsed antigen-presenting cells following autologous transplantation for multiple myeloma may be associated with prolonged survival. American journal of hematology 2009, 84(12):799-802.
208. Yi Q, Szmania S, Freeman J, Qian J, Rosen N A, Viswamitra S, Cottler-Fox M, Barlogie B, Tricot G, van Rhee F: Optimizing dendritic cell-based immunotherapy in multiple myeloma: intranodal injections of idiotype-pulsed CD40 ligand-matured vaccines led to induction of type-1 and cytotoxic T-cell immune responses in patients. British journal of haematology 2010, 150(5):554-564.
209. Rollig C, Schmidt C, Bornhauser M, Ehninger G, Schmitz M, Auffermann-Gretzinger S: Induction of cellular immune responses in patients with stage-I multiple myeloma after vaccination with autologous idiotype-pulsed dendritic cells. Journal of immunotherapy 2011, 34(1):100-106.
210. Zahradova L, Mollova K, Ocadlikova D, Kovarova L, Adam Z, Krejci M, Pour L, Krivanova A, Sandecka V, Hajek R: Efficacy and safety of Id-protein-loaded dendritic cell vaccine in patients with multiple myeloma—phase I I study results. Neoplasms 2012, 59(4):440-449.
211. Timmerman J M, Czerwinski D K, Davis T A, Hsu F J, Benike C, Hao Z M, Taidi B, Rajapaksa R, Caspar C B, Okada C Y et al: Idiotype-pulsed dendritic cell vaccination for B-cell lymphoma: clinical and immune responses in 35 patients. Blood 2002, 99(5):1517-1526.
212. Maier T, Tun-Kyi A, Tassis A, Jungius K P, Burg G, Dummer R, Nestle F O: Vaccination of patients with cutaneous T-cell lymphoma using intranodal injection of autologous tumor-lysate-pulsed dendritic cells. Blood 2003, 102(7):2338-2344.
213. Di Nicola M, Zappasodi R, Carlo-Stella C, Mortarini R, Pupa S M, Magni M, Devizzi L, Matteucci P, Baldassari P, Ravagnani F et al: Vaccination with autologous tumor-loaded dendritic cells induces clinical and immunologic responses in indolent B-cell lymphoma patients with relapsed and measurable disease: a pilot study. Blood 2009, 113(1):18-27.
214. Hus I, Rolinski J, Tabarkiewicz J, Wojas K, Bojarska-Junak A, Greiner J, Giannopoulos K, Dmoszynska A, Schmitt M: Allogeneic dendritic cells pulsed with tumor lysates or apoptotic bodies as immunotherapy for patients with early-stage B-cell chronic lymphocytic leukemia. Leukemia 2005, 19(9):1621-1627.
215. Li L, Giannopoulos K, Reinhardt P, Tabarkiewicz J, Schmitt A, Greiner J, Rolinski J, Hus I, Dmoszynska A, Wiesneth M et al: Immunotherapy for patients with acute myeloid leukemia using autologous dendritic cells generated from leukemic blasts. International journal of oncology 2006, 28(4):855-861.
216. Roddie H, Klammer M, Thomas C, Thomson R, Atkinson A, Sproul A, Waterfall M, Samuel K, Yin J, Johnson P et al: Phase I/I I study of vaccination with dendritic-like leukaemia cells for the immunotherapy of acute myeloid leukaemia. British journal of haematology 2006, 133(2):152-157.
217. Litzow M R, Dietz A B, Bulur P A, Butler G W, Gastineau D A, Hoering A, Fink S R, Letendre L, Padley D J, Paternoster S F et al: Testing the safety of clinical-grade mature autologous myeloid D C in a phase I clinical immunotherapy trial of CML. Cytotherapy 2006, 8(3):290-298.
218. Westermann J, Kopp J, van Lessen A, Hecker A C, Baskaynak G, le Coutre P, Dohner K, Dohner H, Dorken B, Pezzutto A: Vaccination with autologous non-irradiated dendritic cells in patients with bcr/abl+ chronic myeloid leukaemia. British journal of haematology 2007, 137(4):297-306.
219. Hus I, Schmitt M, Tabarkiewicz J, Radej S, Wojas K, Bojarska-Junak A, Schmitt A, Giannopoulos K, Dmoszynska A, Rolinski J: Vaccination of B-CLL patients with autologous dendritic cells can change the frequency of leukemia antigen-specific CD8+ T cells as well as CD4+CD25+FoxP3+ regulatory T cells toward an antileukemia response. Leukemia 2008, 22(5):1007-1017.
220. Palma M, Adamson L, Hansson L, Kokhaei P, Rezvany R, Mellstedt H, Osterborg A, Choudhury A: Development of a dendritic cell-based vaccine for chronic lymphocytic leukemia. Cancer immunology, immunotherapy: CII 2008, 57(11):1705-1710.
221. Van Tendeloo V F, Van de Velde A, Van Driessche A, Cools N, Anguille S, Ladell K, Gostick E, Vermeulen K, Pieters K, Nijs G et al: Induction of complete and molecular remissions in acute myeloid leukemia by Wilms' tumor 1 antigen-targeted dendritic cell vaccination. Proceedings of the National Academy of Sciences of the United States of America 2010, 107(31):13824-13829.
222. Iwashita Y, Tahara K, Goto S, Sasaki A, Kai S, Seike M, Chen C L, Kawano K, Kitano S: A phase I study of autologous dendritic cell-based immunotherapy for patients with unresectable primary liver cancer. Cancer immunology, immunotherapy: CII 2003, 52(3):155-161.
223. Lee W C, Wang H C, Hung C F, Huang P F, Lia C R, Chen M F: Vaccination of advanced hepatocellular carcinoma patients with tumor lysate-pulsed dendritic cells: a clinical trial. Journal of immunotherapy 2005, 28(5):496-504.
224. Butterfield L H, Ribas A, Dissette V B, Lee Y, Yang J Q, De la Rocha P, Duran S D, Hernandez J, Seja E, Potter D M et al: A phase I/I I trial testing immunization of hepatocellular carcinoma patients with dendritic cells pulsed with four alpha-fetoprotein peptides. Clinical cancer research: an official journal of the American Association for Cancer Research 2006, 12(9):2817-2825.
225. Palmer D H, Midgley R S, Mirza N, Ton E E, Ahmed F, Steele J C, Steven N M, Kerr D J, Young L S, Adams D H: A phase I I study of adoptive immunotherapy using dendritic cells pulsed with tumor lysate in patients with hepatocellular carcinoma. Hepatology 2009, 49(1):124-132.
226. El Ansary M, Mogawer S, Elhamid S A, Alwakil S, Aboelkasem F, Sabaawy H E, Abdelhalim O: Immunotherapy by autologous dendritic cell vaccine in patients with advanced HCC. Journal of cancer research and clinical oncology 2013, 139(1):39-48.
227. Tada F, Abe M, Hirooka M, Ikeda Y, Hiasa Y, Lee Y, Jung N C, Lee W B, Lee H S, Bae Y S et al: Phase I/I I study of immunotherapy using tumor antigen-pulsed dendritic cells in patients with hepatocellular carcinoma. International journal of oncology 2012, 41(5): 1601-1609.
228. Ueda Y, Itoh T, Nukaya I, Kawashima I, Okugawa K, Yano Y, Yamamoto Y, Naitoh K, Shimizu K, Imura K et al: Dendritic cell-based immunotherapy of cancer with carcinoembryonic antigen-derived, HLA-A24-restricted CTL epitope: Clinical outcomes of 18 patients with metastatic gastrointestinal or lung adenocarcinomas. International journal of oncology 2004, 24(4):909-917.
229. Hirschowitz E A, Foody T, Kryscio R, Dickson L, Sturgill J, Yannelli J: Autologous dendritic cell vaccines for non-small-cell lung cancer. Journal of clinical oncology: official journal of the American Society of Clinical Oncology 2004, 22(14):2808-2815.
230. Chang G C, Lan H C, Juang S H, Wu Y C, Lee H C, Hung Y M, Yang H Y, Whang-Peng J, Liu K J: A pilot clinical trial of vaccination with dendritic cells pulsed with autologous tumor cells derived from malignant pleural effusion in patients with late-stage lung carcinoma. Cancer 2005, 103(4):763-771.
231. Yannelli J R, Sturgill J, Foody T, Hirschowitz E: The large scale generation of dendritic cells for the immunization of patients with non-small cell lung cancer (NSCLC). Lung cancer 2005, 47(3):337-350.
232. Ishikawa A, Motohashi S, Ishikawa E, Fuchida H, Higashino K, Otsuji M, lizasa T, Nakayama T, Taniguchi M, Fujisawa T: A phase I study of alpha-galactosylceramide (KRN7000)-pulsed dendritic cells in patients with advanced and recurrent non-small cell lung cancer. Clinical cancer research: an official journal of the American Association for Cancer Research 2005, 11(5):1910-1917.
233. Antonia S J, Mirza N, Fricke I, Chiappori A, Thompson P, Williams N, Bepler G, Simon G, Janssen W, Lee J H et al: Combination of p53 cancer vaccine with chemotherapy in patients with extensive stage small cell lung cancer. Clinical cancer research: an official journal of the American Association for Cancer Research 2006, 12(3 Pt 1):878-887.
234. Perrot I, Blanchard D, Freymond N, Isaac S, Guibert B, Pacheco Y, Lebecque S: Dendritic cells infiltrating human non-small cell lung cancer are blocked at immature stage. Journal of immunology 2007, 178(5):2763-2769.
235. Hirschowitz E A, Foody T, Hidalgo G E, Yannelli J R: Immunization of NSCLC patients with antigen-pulsed immature autologous dendritic cells. Lung cancer 2007, 57(3):365-372.
236. Baratelli F, Takedatsu H, Hazra S, Peebles K, Luo J, Kurimoto P S, Zeng G, Batra R K, Sharma S, Dubinett S M et al: Pre-clinical characterization of GMP grade CCL21-gene modified dendritic cells for application in a phase I trial in non-small cell lung cancer. Journal of translational medicine 2008, 6:38.
237. Hegmans J P, Veltman J D, Lambers M E, de Vries I J, Figdor C G, Hendriks R W, Hoogsteden H C, Lambrecht B N, Aerts J G: Consolidative dendritic cell-based immunotherapy elicits cytotoxicity against malignant mesothelioma. American journal of respiratory and critical care medicine 2010, 181(12):1383-1390.
238. Um S J, Choi Y J, Shin H J, Son C H, Park Y S, Roh M S, Kim Y S, Kim Y D, Lee S K, Jung M H et al: Phase I study of autologous dendritic cell tumor vaccine in patients with non-small cell lung cancer. Lung cancer 2010, 70(2):188-194.
239. Chiappori A A, Soliman H, Janssen W E, Antonia S J, Gabrilovich D I: INGN-225: a dendritic cell-based p53 vaccine (Ad.p53-D C) in small cell lung cancer: observed association between immune response and enhanced chemotherapy effect. Expert opinion on biological therapy 2010, 10(6):983-991.
240. Perroud M W, Jr., Honma H N, Barbeiro A S, Gilli S C, Almeida M T, Vassallo J, Saad S T, Zambon L: Mature autologous dendritic cell vaccines in advanced non-small cell lung cancer: a phase I pilot study. Journal of experimental & clinical cancer research: C R 2011, 30:65.
241. Skachkova O V, Khranovska N M, Gorbach O I, Svergun N M, Sydor R I, Nikulina V V: Immunological markers of anti-tumor dendritic cells vaccine efficiency in patients with non-small cell lung cancer. Experimental oncology 2013, 35(2):109-113.
242. Hernando J J, Park T W, Kubler K, Offergeld R, Schlebusch H, Bauknecht T: Vaccination with autologous tumour antigen-pulsed dendritic cells in advanced gynaecological malignancies: clinical and immunological evaluation of a phase I trial. Cancer immunology, immunotherapy: CII 2002, 51(1):45-52.
243. Rahma O E, Ashtar E, Czystowska M, Szajnik M E, Wieckowski E, Bernstein S, Herrin V E, Shams M A, Steinberg S M, Merino M et al: A gynecologic oncology group phase I I trial of two p53 peptide vaccine approaches: subcutaneous injection and intravenous pulsed dendritic cells in high recurrence risk ovarian cancer patients. Cancer immunology, immunotherapy: CII 2012, 61(3):373-384.
244. Chu C S, Boyer J, Schullery D S, Gimotty P A, Gamerman V, Bender J, Levine B L, Coukos G, Rubin S C, Morgan M A et al: Phase VII randomized trial of dendritic cell vaccination with or without cyclophosphamide for consolidation therapy of advanced ovarian cancer in first or second remission. Cancer immunology, immunotherapy: CII 2012, 61(5):629-641.
245. Kandalaft L E, Chiang C L, Tanyi J, Motz G, Balint K, Mick R, Coukos G: A Phase I vaccine trial using dendritic cells pulsed with autologous oxidized lysate for recurrent ovarian cancer. Journal of translational medicine 2013, 11:149.
246. Lepisto A J, Moser A J, Zeh H, Lee K, Bartlett D, McKolanis J R, Geller B A, Schmotzer A, Potter D P, Whiteside T et al: A phase VII study of a MUC1 peptide pulsed autologous dendritic cell vaccine as adjuvant therapy in patients with resected pancreatic and biliary tumors. Cancer therapy 2008, 6(B):955-964.
247. Rong Y, Qin X, Jin D, Lou W, Wu L, Wang D, Wu W, Ni X, Mao Z, Kuang T et al: A phase I pilot trial of MUC1-peptide-pulsed dendritic cells in the treatment of advanced pancreatic cancer. Clinical and experimental medicine 2012, 12(3): 173-180.
248. Endo H, Saito T, Kenjo A, Hoshino M, Terashima M, Sato T, Anazawa T, Kimura T, Tsuchiya T, Irisawa A et al: Phase I trial of preoperative intratumoral injection of immature dendritic cells and OK-432 for resectable pancreatic cancer patients. Journal of hepatobiliary—pancreatic sciences 2012, 19(4):465-475.

1. A dendritic cell capable of stimulating natural killer cell activity and/or natural killer cell number in a host, said dendritic cell generated by the steps of: a) obtaining a monocytic cell; b) treating said monocytic cell in a manner to induce differentiation along the dendritic cell lineage; and c) exposing said dendritic cell to a stimulator of innate immune function for a sufficient time and concentration to endow said dendritic cell ability to activate NK cells.

2.-10. (canceled)

11. The dendritic cell of claim 1, wherein said dendritic cell is not adherent to plastic.

12. The dendritic cell of claim 1, wherein said dendritic cell is generated by culturing a monocyte in the presence of GM-CSF and interleukin-4.

13.-37. (canceled)

38. The dendritic cell of claim 1, wherein the dendritic cells are generated from extracting monocytic cells from a tissue source of bone marrow and said bone marrow cells are treated with an agent capable of killing cells expressing antigens which are not expressed on dendritic precursor cells by contacting the bone marrow with antibodies specific for antigens not present on dendritic precursor cells in a medium comprising complement.

39. (canceled)

40. The dendritic cell of claim 38, wherein the bone marrow is cultured with GM-CSF at a concentration of about 500-1000 U/ml.

41.-240. (canceled)

241. The dendritic cell of claim 38, wherein the bone marrow is cultured with IL-4 at a concentration of about 10-1000 U/ml.

242. The dendritic cell of claim 1, wherein said stimulator of innate immune function is allogeneic lymphocytes.

243. A method of protecting against and/or treating coronavirus comprising the steps of a) obtaining a monocytic cell; b) treating said monocytic cell in a manner to induce differentiation along the dendritic cell lineage; and c) exposing said dendritic cell to a stimulator of innate immune function for a sufficient time and concentration to endow said dendritic cell ability to activate NK cells.

244. The method of claim 243, wherein said dendritic cell is not adherent to plastic.

245. The method of claim 244, wherein said dendritic cell is generated by culturing a monocyte in the presence of GM-CSF and interleukin-4.

246. The method of claim 243, wherein the dendritic cells are generated from extracting monocytic cells from a tissue source of bone marrow and said bone marrow cells are treated with an agent capable of killing cells expressing antigens which are not expressed on dendritic precursor cells by contacting the bone marrow with antibodies specific for antigens not present on dendritic precursor cells in a medium comprising complement.

247. The method of claim 246, wherein the bone marrow is cultured with GM-CSF at a concentration of about 50-1000 U/ml.

248. The method of claim 246, wherein the bone marrow is cultured with IL-4 at a concentration of about 10-1000 U/ml.

249. The method of claim 243, wherein said dendritic cell is pulsed with peptides selected from a group comprising of: SARS-CoV-2 spike protein in its entirety and/or spike protein epitope comprising residues 274-306, and/or spike protein epitope comprising residues 510-586, and/or spike protein epitope comprising residues 587-628, and/or spike protein epitope comprising residues 784-803, and/or spike protein epitope comprising residues 870-893.

250. The method of claim 243, wherein said dendritic cell is pulsed with peptides selected from a group comprising of: SGSGPATVCGPKKSTNLVKNKC (SEQ ID NO: 1), SGSGKSTNLVKNKCVNFNFNGL(SEQ ID NO: 2), SGSGKCVNFNFNGLTGTGVLTE(SEQ ID NO: 3), SGSGGLTGTGVLTESNKKFLPF(SEQ ID NO: 4), SGSGTESNKKFLPFQQFGRDIA(SEQ ID NO: 5), SGSGNFSQILPDPSKPSKRSFI(SEQ ID NO: 6), SGSGPSKPSKRSFIEDLLFNKV(SEQ ID NO: 7), SGSGFIEDLLFNKVTLADAGFI(SEQ ID NO: 8),

251. A method of inhibiting/treating coronavirus infection by administration of leucocyte extract at a concentration and frequency sufficient to induce generation of natural killer cell activity.

252. The method of claim 251, further comprising administration of SARS-CoV-2 spike protein in its entirety and/or spike protein epitope comprises residues 274-306, and/or spike protein epitope comprising residues 510-586, and/or spike protein epitope comprising residues 587-628, and/or spike protein epitope comprising residues 784-803, and/or spike protein epitope comprising residues 870-893.
